(19) United States
(12) Patent Application Publication
(10) Pub. No.: US 2026/0062463 A1
(43) Pub. Date: Mar. 5, 2026

(54) NANOBODY, POLYPEPTIDE CONTAINING SAID NANOBODY AND USE THEREOF

(71) Applicant: KANGYUAN BIOMEDICAL TECH. (DALIAN) CO.,LTD, Dalian (CN)

(72) Inventors: Yumeng WANG, Dalian (CN); Chundong HUANG, Dalian (CN); Da LI, Dalian (CN); Fu CHEN, Dalian (CN)

(73) Assignee: KANGYUAN BIOMEDICAL TECH. (DALIAN) CO.,LTD, Dalian (CN)

(21) Appl. No.: 19/317,034

(22) Filed: Sep. 2, 2025

Related U.S. Application Data
(63) Continuation of application No. PCT/CN2023/123661, filed on Oct. 10, 2023.

(30) Foreign Application Priority Data
Jun. 13, 2023 (CN) 202310699491.2

(51) Int. Cl. C07K 16/08

(52) U.S. Cl. CPC C07K 16/081; C07K 2317/24; C07K 2317/35; C07K 2317/565; C07K 2317/567; C07K 2317/569

(57) ABSTRACT

Provided herein are nanobodies, polypeptides comprising the same, and uses thereof. The nanobody's variable region includes 3 complementarity determining regions (CDRs) and framework regions (FRs), wherein the CDRs are as follows: CDR1: Ser-Gly-Xaa11-Xaa12-Phe-Xaa13-Xaa14-Asn-Xaa15 (Formula I); CDR2: Xaa21-Thr-Xaa22-Xaa23-Gly-Xaa24-Thr (Formula II); CDR3: His-Xaa31-Asp-Glu-Xaa32-Arg-Xaa33-Ser-Xaa34-Trp-Thr-Thr-Ser-Asn-Xaa35 (Formula III). The nanobodies and their polypeptides exhibit high affinity and activity, specifically recognizing and binding AAV. Affinity agents prepared therefrom have strong AAV adsorption capacity, suitable for AAV affinity chromatography to facilitate its industrial application. They are also applicable to AAV detection, enabling simultaneous detection of empty capsids and viral particles.

### FIELD OF THE INVENTION

The present invention relates to amino acid sequences that are directed against/and or that can specifically bind (as defined herein) Adeno-Associated Virus (AAV), as well as to compounds or constructs, and in particular proteins and polypeptides, that comprise or essentially consist of one or more such amino acid sequences (also referred to herein as “amino acid sequences of the invention”, “compounds of the invention”, “constructs of the invention” and “polypeptides of the invention”, respectively).

The present invention further encompasses nucleic acids encoding the aforementioned amino acid sequences and polypeptides (hereinafter referred to as “nucleic acids of the invention” or “nucleotide sequences of the invention”), methods for preparing said amino acid sequences and polypeptides, host cells expressing or capable of expressing such amino acid sequences or polypeptides, compositions (particularly those for the specific recognition of AAV) comprising said amino acid sequences, polypeptides, nucleic acids, and/or host cells, and uses of said amino acid sequences, polypeptides, nucleic acids, host cells, and/or compositions (particularly in adsorbents for the purification of AAV, wherein the adsorbent selectively captures AAV from complex biological matrices, and in the preparation of detection kits for AAV, wherein the nanobodies or polypeptides serve as specific binding agents for AAV diagnosis or quantification).

### BACKGROUND ART

The adeno-associated virus (AAV), a member of the Dependoparvovirus genus within the Parvoviridae family, possesses a non-enveloped, icosahedral structure. With a diameter ranging from 20 to 26 nm, AAV contains a linear single-stranded DNA genome of approximately 4.7 to 6 kb in size. As a defective virus, AAV relies on a helper virus, commonly adenovirus, to facilitate its replication process. Notably, AAV does not cause disease in humans, and seroprevalence studies indicate that around 80% of the population exhibit antibodies against AAV.

Recombinant adeno-associated virus (rAAV) vectors are derived from the non-pathogenic wild-type AAV. Owing to their favorable safety profile, broad tropism for both dividing and non-dividing cells, low immunogenicity, and the ability to enable long-term expression of therapeutic transgenes in vivo, rAAVs have emerged as one of the most promising vectors for gene transfer. These attributes have positioned rAAVs at the forefront of preclinical and clinical research in gene therapy and vaccine development. Their applications span across diverse research areas, including the investigation of gene functions through in vitro and in vivo experiments, the establishment of disease models, gene knockout studies, gene therapy trials, and vaccine research. The versatile and advantageous features of rAAVs make them invaluable tools in advancing biomedical research and translating discoveries into potential clinical treatments.

During the upstream production of recombinant adeno-associated virus (rAAV) in cells, the resulting lysate comprises not only the desired product but also a variety of impurities. These contaminants encompass nucleic acids, residual components from the culture medium, and host cell proteins (HCPs). While residual DNA can be effectively addressed through nuclease treatment, the removal of HCPs presents a considerably more intricate challenge. This process typically demands a multi-step approach, which can lead to reduced overall yields, complicate process development efforts, and pose substantial hurdles for downstream purification procedures.

An additional layer of complexity arises from the existence of multiple AAV serotypes, designated AAV1 through AAV10. These serotypes exhibit distinct capsid protein spatial configurations, sequences, and tissue-specific tropisms. Consequently, they demonstrate varying infection efficiencies across different tissues and cells, and interact with diverse cell surface receptors. Due to these differences, the development of a one-size-fits-all affinity chromatography strategy remains elusive.

Traditional downstream processing workflows for rAAV purification necessitate a sequence of diverse treatment modalities. These often include cesium chloride or iodixanol gradient centrifugation, as well as multiple chromatography steps. However, such approaches are far from optimal. When numerous steps are required, even if each individual step achieves a high level of efficiency, the cumulative effect can result in a pronounced decrease in the overall final yield. Moreover, a primary disadvantage of these conventional methods is their limited scalability. They are not well-suited for direct purification of viruses from large-volume lysates and are generally restricted to applications of a research nature. Consequently, there is a pressing need for more efficient and scalable downstream processes to meet the demands of rAAV production for therapeutic applications.

Recent advances in affinity chromatography have streamlined AAV purification workflows, reducing the number of steps and consequently enhancing yields while expediting processing times. For instance, heparin affinity columns have proven effective for purifying rAAV3 and rAAV6, whereas mucin affinity columns are suitable for rAAV1, rAAV4, rAAV5, and rAAV6. Additionally, antibody affinity columns have been employed for both detection and purification of rAAV. Examples include monoclonal antibodies specific to AAVXL32.1 and polyclonal antibodies targeting the AAV9 capsid protein.

Nevertheless, the industrial application of traditional antibody-based methods has been hindered by the high costs associated with antibody preparation and the large size of antibodies, which limits their coupling efficiency to chromatography matrices. In contrast, camelid nanobodies, known for their high specificity and stability, have shown great potential. Nanobody-based affinity resins, such as AVB Sepharose™ and POROS™ CaptureSelect AAVX, have demonstrated excellent performance in column chromatography, offering a promising solution for industrial-scale AAV purification. However, it is important to note that nanobodies obtained through different screening methods may exhibit considerable variability in terms of affinity, stability, and binding spectrum. This variability can impact the overall performance and reliability of the purification process.

### SUMMARY OF THE INVENTION

A specific, but non-limiting object of the present invention is to provide amino acid sequences, polypeptides, and compositions that can effectively address the drawbacks of existing AAV enrichment, purification, and detection methods, including cumbersome antibody preparation protocols, prohibitive costs, and suboptimal antibody affinity, stability, and binding spectra.

The technical solution presented in this application is structured as follows:

According to one aspect, the present disclosure provides VHH sequences capable of specifically binding to AAV, comprising complementarity-determining region 1 (CDR1), CDR2, and CDR3, wherein:
The CDR1 conforms to the definition delineated in the following formula (I): Ser-Gly-Xaa₁₁-Xaa₁₂-Phe-Xaa₁₃-Xaa₁₄-Asn-Xaa₁₅, (I);
The CDR2 conforms to the definition delineated in the following formula (II): Xaa₂₁-Thr-Xaa₂₂-Xaa₂₃-Gly-Xaa₂₄-Thr, (II);
The CDR3 conforms to the definition delineated in the following formula (III): His-Xaa₃₁-Asp-Glu-Xaa₃₂-Arg-Xaa₃₃-Ser-Xaa₃₄-Trp-Thr-Thr-Ser-Asn-Xaa₃₅, (III).

Wherein:
Xaa₁₁ is Arg, Ser, or Thr;
Xaa₁₂ is Ala, Gly, His, Ile, Met, Asn, Arg, Ser, or Thr;
Xaa₁₃ is Ile, Arg, Ser, Thr, or Val;
Xaa₁₄ is Ala, Ile, or Leu;
Xaa₁₅ is Ala, Ile, Leu, Ser, Thr, or Val;
Xaa₂₁ is Phe, Ile, Leu, or Val;
Xaa₂₂ is Pro, Arg, or Ser;
Xaa₂₃ is Ala, Asp, or Gly;
Xaa₂₄ is Asp, Gly, Asn, Ser, Thr, or Val;
Xaa₃₁ is Ile, Leu, or Val;
Xaa₃₂ is Ile, Leu, or Val;
Xaa₃₃ is Asp, Glu, or Gln;
Xaa₃₄ is Gly, Ile, Ser, or Thr;
Xaa₃₅ is Ile or Leu.

Preferably, in the CDR1,
Xaa₁₁ is Arg, Xaa₁₂ is Ser, Xaa₁₃ is Ser, Xaa₁₄ is Ile, and Xaa₁₅ is Thr; or
Xaa₁₁ is Ser, Xaa₁₂ is Arg, Xaa₁₃ is Ser, Xaa₁₄ is Ile, and Xaa₁₅ is Thr; or
Xaa₁₁ is Arg, Xaa₁₂ is His, Xaa₁₃ is Ser, Xaa₁₄ is Ile, and Xaa₁₅ is Thr; or
Xaa₁₁ is Arg, Xaa₁₂ is Ala, Xaa₁₃ is Ser, Xaa₁₄ is Ile, and Xaa₁₅ is Thr; or
Xaa₁₁ is Ser, Xaa₁₂ is Ile, Xaa₁₃ is Ser, Xaa₁₄ is Ile, and Xaa₁₅ is Ser; or
Xaa₁₁ is Ser, Xaa₁₂ is Arg, Xaa₁₃ is Ser, Xaa₁₄ is Ile, and Xaa₁₅ is Ser; or
Xaa₁₁ is Ser, Xaa₁₂ is Ile, Xaa₁₃ is Ser, Xaa₁₄ is Ile, and Xaa₁₅ is Leu; or
Xaa₁₁ is Ser, Xaa₁₂ is Thr, Xaa₁₃ is Ser, Xaa₁₄ is Ile, and Xaa₁₅ is Thr; or
Xaa₁₁ is Ser, Xaa₁₂ is Gly, Xaa₁₃ is Ser, Xaa₁₄ is Ile, and Xaa₁₅ is Thr; or
Xaa₁₁ is Ser, Xaa₁₂ is Ala, Xaa₁₃ is Ser, Xaa₁₄ is Ile, and Xaa₁₅ is Thr; or
Xaa₁₁ is Ser, Xaa₁₂ is Asn, Xaa₁₃ is Arg, Xaa₁₄ is Ile, and Xaa₁₅ is Thr; or
Xaa₁₁ is Ser, Xaa₁₂ is Asn, Xaa₁₃ is Ser, Xaa₁₄ is Ile, and Xaa₁₅ is Thr; or
Xaa₁₁ is Ser, Xaa₁₂ is Ser, Xaa₁₃ is Thr, Xaa₁₄ is Ile, and Xaa₁₅ is Thr; or
Xaa₁₁ is Ser, Xaa₁₂ is Ser, Xaa₁₃ is Ser, Xaa₁₄ is Ile, and Xaa₁₅ is Thr; or
Xaa₁₁ is Arg, Xaa₁₂ is Gly, Xaa₁₃ is Ser, Xaa₁₄ is Ile, and Xaa₁₅ is Thr; or
Xaa₁₁ is Ser, Xaa₁₂ is Ile, Xaa₁₃ is Ser, Xaa₁₄ is Ile, and Xaa₁₅ is Thr; or
Xaa₁₁ is Ser, Xaa₁₂ is His, Xaa₁₃ is Ser, Xaa₁₄ is Ile, and Xaa₁₅ is Thr; or
Xaa₁₁ is Arg, Xaa₁₂ is Ile, Xaa₁₃ is Ser, Xaa₁₄ is Ile, and Xaa₁₅ is Thr; or
Xaa₁₁ is Ser, Xaa₁₂ is Met, Xaa₁₃ is Ser, Xaa₁₄ is Ile, and Xaa₁₅ is Thr; or
Xaa₁₁ is Ser, Xaa₁₂ is Ser, Xaa₁₃ is Val, Xaa₁₄ is Ile, and Xaa₁₅ is Thr; or
Xaa₁₁ is Ser, Xaa₁₂ is Ile, Xaa₁₃ is Ile, Xaa₁₄ is Ile, and Xaa₁₅ is Thr; or
Xaa₁₁ is Ser, Xaa₁₂ is His, Xaa₁₃ is Ser, Xaa₁₄ is Leu, and Xaa₁₅ is Thr; or
Xaa₁₁ is Ser, Xaa₁₂ is Thr, Xaa₁₃ is Ser, Xaa₁₄ is Leu, and Xaa₁₅ is Thr; or
Xaa₁₁ is Ser, Xaa₁₂ is Arg, Xaa₁₃ is Ser, Xaa₁₄ is Leu, and Xaa₁₅ is Thr; or
Xaa₁₁ is Ser, Xaa₁₂ is Ser, Xaa₁₃ is Ser, Xaa₁₄ is Ile, and Xaa₁₅ is Ala; or
Xaa₁₁ is Arg, Xaa₁₂ is Arg, Xaa₁₃ is Thr, Xaa₁₄ is Ile, and Xaa₁₅ is Thr; or
Xaa₁₁ is Arg, Xaa₁₂ is Asn, Xaa₁₃ is Ser, Xaa₁₄ is Leu, and Xaa₁₅ is Thr; or
Xaa₁₁ is Arg, Xaa₁₂ is Ala, Xaa₁₃ is Ser, Xaa₁₄ is Leu, and Xaa₁₅ is Thr; or
Xaa₁₁ is Ser, Xaa₁₂ is Ile, Xaa₁₃ is Thr, Xaa₁₄ is Ile, and Xaa₁₅ is Ser; or
Xaa₁₁ is Ser, Xaa₁₂ is Met, Xaa₁₃ is Ser, Xaa₁₄ is Leu, and Xaa₁₅ is Thr; or
Xaa₁₁ is Thr, Xaa₁₂ is Met, Xaa₁₃ is Ser, Xaa₁₄ is Leu, and Xaa₁₅ is Thr; or
Xaa₁₁ is Thr, Xaa₁₂ is Arg, Xaa₁₃ is Ser, Xaa₁₄ is Ile, and Xaa₁₅ is Thr; or
Xaa₁₁ is Ser, Xaa₁₂ is Arg, Xaa₁₃ is Ile, Xaa₁₄ is Ile, and Xaa₁₅ is Thr; or
Xaa₁₁ is Ser, Xaa₁₂ is Ile, Xaa₁₃ is Val, Xaa₁₄ is Ile, and Xaa₁₅ is Thr; or
Xaa₁₁ is Ser, Xaa₁₂ is Ser, Xaa₁₃ is Ser, Xaa₁₄ is Ala, and Xaa₁₅ is Thr; or
Xaa₁₁ is Ser, Xaa₁₂ is Ile, Xaa₁₃ is Ser, Xaa₁₄ is Ala, and Xaa₁₅ is Thr; or
Xaa₁₁ is Ser, Xaa₁₂ is Arg, Xaa₁₃ is Ser, Xaa₁₄ is Ile, and Xaa₁₅ is Ile; or
Xaa₁₁ is Ser, Xaa₁₂ is His, Xaa₁₃ is Thr, Xaa₁₄ is Ile, and Xaa₁₅ is Ser; or
Xaa₁₁ is Ser, Xaa₁₂ is Ile, Xaa₁₃ is Ser, Xaa₁₄ is Leu, and Xaa₁₅ is Ala; or
Xaa₁₁ is Ser, Xaa₁₂ is Ile, Xaa₁₃ is Ser, Xaa₁₄ is Leu, and Xaa₁₅ is Ser; or
Xaa₁₁ is Thr, Xaa₁₂ is Ile, Xaa₁₃ is Ser, Xaa₁₄ is Leu, and Xaa₁₅ is Val; or
Xaa₁₁ is Thr, Xaa₁₂ is Asn, Xaa₁₃ is Ser, Xaa₁₄ is Ile, and Xaa₁₅ is Val.

Preferably, in the CDR2,
Xaa₂₁ is Val, Xaa₂₂ is Arg, Xaa₂₃ is Gly, and Xaa₂₄ is Ser; or
Xaa₂₁ is Val, Xaa₂₂ is Arg, Xaa₂₃ is Gly, and Xaa₂₄ is Gly; or
Xaa₂₁ is Ile, Xaa₂₂ is Arg, Xaa₂₃ is Gly, and Xaa₂₄ is Asn; or
Xaa₂₁ is Ile, Xaa₂₂ is Arg, Xaa₂₃ is Gly, and Xaa₂₄ is Ser; or
Xaa₂₁ is Val, Xaa₂₂ is Arg, Xaa₂₃ is Gly, and Xaa₂₄ is Asn; or
Xaa₂₁ is Ile, Xaa₂₂ is Pro, Xaa₂₃ is Gly, and Xaa₂₄ is Asn; or
Xaa₂₁ is Val, Xaa₂₂ is Arg, Xaa₂₃ is Gly, and Xaa₂₄ is Val; or
Xaa₂₁ is Val, Xaa₂₂ is Arg, Xaa₂₃ is Asp, and Xaa₂₄ is Ser; or
Xaa₂₁ is Val, Xaa₂₂ is Arg, Xaa₂₃ is Gly, and Xaa₂₄ is Thr; or
Xaa₂₁ is Phe, Xaa₂₂ is Arg, Xaa₂₃ is Ala, and Xaa₂₄ is Asn; or
Xaa₂₁ is Val, Xaa₂₂ is Arg, Xaa₂₃ is Gly, and Xaa₂₄ is Asp; or
Xaa₂₁ is Leu, Xaa₂₂ is Arg, Xaa₂₃ is Gly, and Xaa₂₄ is Ser; or
Xaa₂₁ is Val, Xaa₂₂ is Pro, Xaa₂₃ is Ala, and Xaa₂₄ is Ser; or
Xaa₂₁ is Val, Xaa₂₂ is Ser, Xaa₂₃ is Ala, and Xaa₂₄ is Ser; or
Xaa₂₁ is Val, Xaa₂₂ is Ser, Xaa₂₃ is Gly, and Xaa₂₄ is Gly; or
Xaa₂₁ is Val, Xaa₂₂ is Arg, Xaa₂₃ is Asp, and Xaa₂₄ is Gly; or
Xaa₂₁ is Ile, Xaa₂₂ is Arg, Xaa₂₃ is Gly, and Xaa₂₄ is Gly; or
Xaa₂₁ is Val, Xaa₂₂ is Arg, Xaa₂₃ is Ala, and Xaa₂₄ is Ser; or
Xaa₂₁ is Val, Xaa₂₂ is Pro, Xaa₂₃ is Gly, and Xaa₂₄ is Ser; or
Xaa₂₁ is Phe, Xaa₂₂ is Arg, Xaa₂₃ is Gly, and Xaa₂₄ is Asn; or
Xaa₂₁ is Ile, Xaa₂₂ is Ser, Xaa₂₃ is Gly, and Xaa₂₄ is Ser.

### Preferably, in the CDR3:

Xaa₃₁ is Ile, Xaa₃₂ is Val, Xaa₃₃ is Gln, Xaa₃₄ is Ser, and Xaa₃₅ is Ile; or
Xaa₃₁ is Val, Xaa₃₂ is Val, Xaa₃₃ is Gln, Xaa₃₄ is Ser, and Xaa₃₅ is Ile; or
Xaa₃₁ is Val, Xaa₃₂ is Val, Xaa₃₃ is Glu, Xaa₃₄ is Ser, and Xaa₃₅ is Leu; or
Xaa₃₁ is Val, Xaa₃₂ is Val, Xaa₃₃ is Gln, Xaa₃₄ is Ser, and Xaa₃₅ is Leu; or
Xaa₃₁ is Val, Xaa₃₂ is Val, Xaa₃₃ is Asp, Xaa₃₄ is Ser, and Xaa₃₅ is Ile; or
Xaa₃₁ is Leu, Xaa₃₂ is Val, Xaa₃₃ is Glu, Xaa₃₄ is Ser, and Xaa₃₅ is Leu; or
Xaa₃₁ is Val, Xaa₃₂ is Ile, Xaa₃₃ is Glu, Xaa₃₄ is Ser, and Xaa₃₅ is Leu; or
Xaa₃₁ is Ile, Xaa₃₂ is Ile, Xaa₃₃ is Glu, Xaa₃₄ is Ser, and Xaa₃₅ is Leu; or
Xaa₃₁ is Val, Xaa₃₂ is Ile, Xaa₃₃ is Gln, Xaa₃₄ is Ser, and Xaa₃₅ is Leu; or
Xaa₃₁ is Leu, Xaa₃₂ is Val, Xaa₃₃ is Gln, Xaa₃₄ is Ser, and Xaa₃₅ is Ile; or
Xaa₃₁ is Leu, Xaa₃₂ is Val, Xaa₃₃ is Gln, Xaa₃₄ is Ser, and Xaa₃₅ is Leu; or
Xaa₃₁ is Ile, Xaa₃₂ is Val, Xaa₃₃ is Gln, Xaa₃₄ is Ser, and Xaa₃₅ is Leu; or
Xaa₃₁ is Val, Xaa₃₂ is Leu, Xaa₃₃ is Gln, Xaa₃₄ is Ser, and Xaa₃₅ is Leu; or
Xaa₃₁ is Leu, Xaa₃₂ is Val, Xaa₃₃ is Gln, Xaa₃₄ is Thr, and Xaa₃₅ is Leu; or
Xaa₃₁ is Val, Xaa₃₂ is Val, Xaa₃₃ is Gln, Xaa₃₄ is Thr, and Xaa₃₅ is Leu; or
Xaa₃₁ is Val, Xaa₃₂ is Val, Xaa₃₃ is Glu, Xaa₃₄ is Thr, and Xaa₃₅ is Leu; or
Xaa₃₁ is Leu, Xaa₃₂ is Val, Xaa₃₃ is Asp, Xaa₃₄ is Ser, and Xaa₃₅ is Leu; or
Xaa₃₁ is Val, Xaa₃₂ is Ile, Xaa₃₃ is Gln, Xaa₃₄ is Ser, and Xaa₃₅ is Ile; or
Xaa₃₁ is Val, Xaa₃₂ is Leu, Xaa₃₃ is Asp, Xaa₃₄ is Ser, and Xaa₃₅ is Leu; or
Xaa₃₁ is Val, Xaa₃₂ is Val, Xaa₃₃ is Asp, Xaa₃₄ is Ser, and Xaa₃₅ is Leu; or
Xaa₃₁ is Val, Xaa₃₂ is Leu, Xaa₃₃ is Glu, Xaa₃₄ is Gly, and Xaa₃₅ is Leu; or
Xaa₃₁ is Val, Xaa₃₂ is Leu, Xaa₃₃ is Glu, Xaa₃₄ is Ser, and Xaa₃₅ is Leu; or
Xaa₃₁ is Val, Xaa₃₂ is Leu, Xaa₃₃ is Gln, Xaa₃₄ is Thr, and Xaa₃₅ is Leu; or
Xaa₃₁ is Ile, Xaa₃₂ is Ile, Xaa₃₃ is Glu, Xaa₃₄ is Thr, and Xaa₃₅ is Leu; or
Xaa₃₁ is Val, Xaa₃₂ is Val, Xaa₃₃ is Glu, Xaa₃₄ is Thr, and Xaa₃₅ is Ile; or
Xaa₃₁ is Val, Xaa₃₂ is Ile, Xaa₃₃ is Glu, Xaa₃₄ is Thr, and Xaa₃₅ is Leu; or
Xaa₃₁ is Val, Xaa₃₂ is Val, Xaa₃₃ is Glu, Xaa₃₄ is Ile, and Xaa₃₅ is Leu; or
Xaa₃₁ is Leu, Xaa₃₂ is Val, Xaa₃₃ is Gln, Xaa₃₄ is Thr, and Xaa₃₅ is Ile; or
Xaa₃₁ is Val, Xaa₃₂ is Leu, Xaa₃₃ is Asp, Xaa₃₄ is Thr, and Xaa₃₅ is Leu; or
Xaa₃₁ is Leu, Xaa₃₂ is Val, Xaa₃₃ is Glu, Xaa₃₄ is Thr, and Xaa₃₅ is Leu; or
Xaa₃₁ is Val, Xaa₃₂ is Val, Xaa₃₃ is Gln, Xaa₃₄ is Thr, and Xaa₃₅ is Ile; or
Xaa₃₁ is Val, Xaa₃₂ is Ile, Xaa₃₃ is Gln, Xaa₃₄ is Thr, and Xaa₃₅ is Leu.

Preferably, the framework region (FR) comprises FR1, FR2, FR3, and FR4, or a homologous sequence thereof with more than 50% sequence identity, more preferably more than 70%, and even more preferably more than 95%.

More preferably, the FRI is defined by formula (IV):

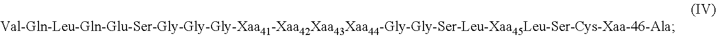

The FR2 is defined by formula (V):

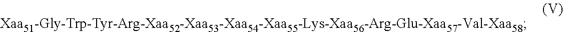

The FR3 is defined by formula (VI):

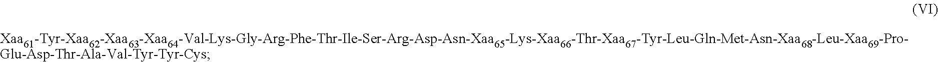

The FR4 is defined by formula (VII):

(VII)
(SEQ ID No: 102)
Trp-Gly-Gln-Gly-Thr-Gln-Val-Thr-Val-Ser-Ser;

Wherein:
Xaa₄₁ independently is Leu or Ala;
Xaa₄₂ independently is Val or Ala;
Xaa₄₃ independently is Gln or His;
Xaa₄₄ independently is Pro, Ala, Ser, or Thr;
Xaa₄₅ independently is Arg or Lys;
Xaa₄₆ independently is Ala, Val, Leu, Ile, or Phe;
Xaa₅₁ independently is Met or Val;
Xaa₅₂ independently is Gln or Arg;
Xaa₅₃ independently is Ala or Arg;
Xaa₅₄ independently is Pro or Ala;
Xaa₅₅ independently is Gly, Pro, Ala, or Arg;
Xaa₅₆ independently is Gln or Trp;
Xaa₅₇ independently is Leu, Phe, Lys, Ile, or Met;
Xaa₅₈ independently is Thr, Ala, Ser, Pro, or Asn;
Xaa₆₁ independently is Asn, Ser, Lys, Thr, or Trp;
Xaa₆₂ independently is Ala or Gly;
Xaa₆₃ independently is Gly, Asp, Glu, or Asn;
Xaa₆₄ independently is Ser or Phe;
Xaa₆₅ independently is Ala, Pro, Thr, Gly, or Asp;
Xaa₆₆ independently is Asn, Asp, Ser, or Thr;
Xaa₆₇ independently is Val, Ile, or Ala;
Xaa₆₈ independently is Ser, Thr, or Asn;
Xaa₆₉ independently is Arg or Gln.

Preferably, FR and CDR are arranged in the order of FR1-CDR1-FR2-CDR2-FR3-CDR3-FR4.

Preferably, the amino acid sequence of the nanobody includes: SEQ ID No: 1, SEQ ID No: 2, SEQ ID No: 3, SEQ ID No: 4, SEQ ID No: 5, SEQ ID No: 6, SEQ ID No: 7, SEQ ID No: 8, SEQ ID No: 9, SEQ ID No: 10, SEQ ID No: 11, SEQ ID No: 12, SEQ ID No: 13, SEQ ID No: 14, SEQ ID No: 15, SEQ ID No: 16, SEQ ID No: 17, SEQ ID No: 18, SEQ ID No: 19, SEQ ID No: 20, SEQ ID No: 21, SEQ ID No: 22, SEQ ID No: 23, SEQ ID No: 24, SEQ ID No: 25, SEQ ID No: 26, SEQ ID No: 27, SEQ ID No: 28, SEQ ID No: 29, SEQ ID No: 30, SEQ ID No: 31, SEQ ID No: 32, SEQ ID No: 33, SEQ ID No: 34, SEQ ID No: 35, SEQ ID No: 36, SEQ ID No: 37, SEQ ID No: 38, SEQ ID No: 39, SEQ ID No: 40, SEQ ID No: 41, SEQ ID No: 42, SEQ ID No: 43, SEQ ID No: 44, SEQ ID No: 45, SEQ ID No: 46, SEQ ID No: 47, SEQ ID No: 48, SEQ ID No: 49, SEQ ID No: 50, SEQ ID No: 51, SEQ ID No: 52, SEQ ID No: 53, SEQ ID No: 54, SEQ ID No: 55, SEQ ID No: 56, SEQ ID No: 57, SEQ ID No: 58, SEQ ID No: 59, SEQ ID No: 60, SEQ ID No: 61, SEQ ID No: 62, SEQ ID No: 63, SEQ ID No: 64, SEQ ID No: 65, SEQ ID No: 66, SEQ ID No: 67, SEQ ID No: 68, SEQ ID No: 69, SEQ ID No: 70, SEQ ID No: 71, SEQ ID No: 72, SEQ ID No: 73, SEQ ID No: 74, SEQ ID No: 75, SEQ ID No: 76, SEQ ID No: 77, SEQ ID No: 78, SEQ ID No: 79, SEQ ID No: 80, SEQ ID No: 81, SEQ ID No: 82, SEQ ID No: 83, SEQ ID No: 84, SEQ ID No: 85, SEQ ID No: 86, SEQ ID No: 87, SEQ ID No: 88, SEQ ID No: 89, SEQ ID No: 90, SEQ ID No: 91, SEQ ID No: 92, SEQ ID No: 93, SEQ ID No: 94, SEQ ID No: 95, SEQ ID No: 96, SEQ ID No: 97, SEQ ID No: 98, SEQ ID No: 99, SEQ ID No: 100, SEQ ID No: 101, SEQ ID No: 102, SEQ ID No: 103, SEQ ID No: 104, SEQ ID No: 105, SEQ ID No: 106, SEQ ID No: 107, SEQ ID No: 108, SEQ ID No: 109, SEQ ID No: 110, SEQ ID No: 111, SEQ ID No: 112, SEQ ID No: 113, SEQ ID No: 114.

Preferably, the nanobody is a humanized nanobody. Preferably, the humanized nanobody includes SEQ ID NOs: 115, 116, 117, 118, 119, and 120.

According to a second aspect, the present invention provides a polypeptide obtained by modifying the N-terminal and/or C-terminal amino acids of the nanobody thereof.

Preferably, the modification of the N-terminal and/or C-terminal amino acids of the nanobody thereof includes:
Method 1: Adding an optional tag or amino acid to the N-terminal and/or C-terminal amino acids of the nanobody.
Method 2: Adding an optional linker to the N-terminal and/or C-terminal amino acids of the nanobody.

Preferably, the tag thereof includes at least one of His-tag, GST-tag, Myc-tag, SUMO-tag, Strep-tag, and Flag-tag; the linker thereof includes at least one of GS linker, IgG hinge linker, IgA hinge linker, and PEG; the protective amino acid includes Ala, Gln, Glu, Met, or any combination of two or more of the aforementioned amino acids.

More preferably, the amino acid sequence of the polypeptide includes SEQ ID NOs: 147, 148, 149, 150, and 151.

According to a third aspect, the present disclosure provides a polypeptide obtained by multivalent synthesis of the above-mentioned nanobody.

Preferably, the multivalent synthesis comprises divalent, trivalent, or tetravalent synthesis;

Preferably, the divalent synthetic amino acid sequence is SEQ ID NO: 121, SEQ ID NO: 122;

Preferably, the amino acid sequence of the humanized divalent polypeptide is SEQ ID NO: 123, SEQ ID NO: 124;

Preferably, the amino acid sequence of the trivalent polypeptide is SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133;

Preferably, the amino acid sequence of the tetravalent polypeptide is SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146.

According to a fourth aspect, the present disclosure provides a nucleic acid, and the nucleic acid encodes the above-mentioned nanobody or the above-mentioned polypeptide.

According to a fifth aspect, the present disclosure provides an expression vector, comprising an expression box of the nucleic acid according to the claims.

According to a sixth aspect, the present disclosure provides a host cell, comprising the expression vector described above in the claims.

In a seventh aspect, the present disclosure provides an application of the nanobody and/or the polypeptide in immunodetection, enrichment and/or purification.

Preferably, the nanobody and/or the polypeptide are used in the preparation of a virus adsorbent, a virus purification kit, and a virus detection kit; further preferably, the virus is AAV.

According to an eighth aspect, the present disclosure provides a virus adsorbent, comprising a carrier matrix and the above-mentioned nanobody and/or the above-mentioned polypeptide.

According to a ninth aspect, the present disclosure provides a virus purification kit, comprising a carrier matrix and the above-mentioned nanobody and/or the above-mentioned polypeptide.

According to a tenth aspect, the present disclosure provides a virus detection kit, comprising a carrier matrix and the above-mentioned nanobody and/or the above-mentioned polypeptide.

According to an eleventh aspect, the present invention provides an AAV detection method, wherein the above-mentioned nanobody and/or the above-mentioned polypeptide are coupled by HRP, and then detected by a direct enzyme immunosorbent assay or a sandwich enzyme immunosorbent assay.

The nanobody of the present invention is a screened anti-AAV nanobody with a new amino acid sequence, the nanobody and the polypeptide thereof have high affinity and activity, can specifically recognize and bind AAV, and the adsorbent prepared from the nanobody and the polypeptide thereof has a very strong adsorption capability on AAV, can be applied to AAV affinity chromatography, and is beneficial to industrial application of the AAV affinity chromatography column. In addition, it can also be applied to the AAV detection field, and the empty capsid and the virus particles can be detected together.

### BRIEF DESCRIPTION OF THE DRAWINGS

FIG. 1 shows purified nanobodies according to Embodiment 1 of the present invention;

FIG. 2 shows binding kinetics curves of nanobodies in Embodiment 5 of the present invention;

FIG. 3 shows schematic diagram of a divalent nanobody according to Embodiment 7 of the present invention;

FIG. 4 shows a standard curve of direct ELISA detection in Embodiment 11 of the present invention;

FIG. 5 shows a standard curve of Sandwich ELISA detection in Embodiment 11 of the present invention.

### DETAILED DESCRIPTION OF EMBODIMENTS OF THE INVENTION

The foregoing and other aspects of the present invention will be described more fully hereinafter, wherein:

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

(1) Unless indicated or defined otherwise, the term “sequence” as used herein (for example in terms like “immunoglobulin sequence”, “antibody sequence”, “variable domain sequence”, “nanobody sequence”, “VHH sequence” or “protein sequence”), should generally be understood to include both the relevant amino acid sequence as well as nucleic acids or nucleotide sequences encoding the same, unless the context requires a more limited interpretation.

(2) Unless indicated or defined otherwise, all methods, steps, techniques, and operations not specifically described are well known to those skilled in the art.

(3) The term “specificity” refers to the number of different types of antigens or antigenic determinants to which a particular antigen-binding molecule or antigen-binding protein. The specificity of an antigen-binding protein can be determined based on affinity and/or avidity, which also describes some preferred techniques for measuring binding between an antigen-binding molecule and the pertinent antigen. The affinity denotes the strength or stability of a molecular interaction. The affinity is commonly given as by the KD, or dissociation constant, which has units of mol/liter (or M). The affinity can also be expressed as an association constant, KA, which equals 1/KD and has units of (mol/liter)⁻¹ (or M⁻¹). In the present specification, the stability of the interaction between two molecules will mainly be expressed in terms of the KD value of their interaction; it being clear to the skilled person that in view of the relation KA=1/KD, specifying the strength of molecular interaction by its KD value can also be used to calculate the corresponding KA value. The smaller the KD value, the stronger the binding strength between the antigen and the antigen-binding molecule. Conversely, the larger the KD value, the weaker the binding strength. Kₐ, the association constant, reflects binding speed: a larger Kₐ indicates faster binding, while a smaller Kₐ indicates slower binding. K_{d}, the dissociation constant, reflects dissociation speed: a larger K_{d} indicates faster dissociation, while a smaller K_{d} indicates slower dissociation. Additionally, the relationship K_{D}=K_{d}/Kₐ holds true.

(4) The amino acid residues of single-domain antibodies (i.e., nanobodies) are numbered according to the Kabat numbering scheme, which has been widely used for the VHH domains from camelids: FRI comprises amino acid residues at positions 1-30, CDR1 comprises residues at positions 31-36, FR2 comprises residues at positions 37-49, CDR2 comprises residues at positions 50-65, FR3 comprises residues at positions 66-94, CDR3 comprises residues at positions 95-102, and FR4 comprises residues at positions 103-113. In this regard, it should be noted that the total number of amino acid residues in each CDR may be different and may not correspond to the total number of amino acid residues indicated by the Kabat numbering scheme (ie, one or more positions according to the Kabat numbering scheme may not be occupied in the actual sequence, or the actual sequence may contain more amino acid residues than the number allowed by the Kabat numbering scheme), as is known in the art. This means that the amino acid residues in the actual sequence may be the same as or different from the actual numbering mode according to the Kabat numbering scheme. In other words, the Kabat numbering scheme does not align with the amino acid residue numbering of the CDRs. Instead, position 1 in the Kabat scheme designates the start of FRI, vice versa, the start of FRI is designated as position 1; position 36 designates the start of FR2, vice versa, the start of FR2 is position 36; position 66 designates the start of FR3, vice versa, the start of FR3 is position 66; position 103 designates the start of FR4, vice versa, the start of FR4 is position 103.

(5) The term “immobilization amount” refers to the total amount of ligand coupled per unit volume of affinity medium (adsorbent).

(6) The term “family” refers to a nanobody sequence family, wherein the sequences binds the same antigen, has the same number of amino acids, and exhibits an amino acid sequence identity of more than 70%.

The amino acid sequence of the nanobody of the present invention substantially comprises complementary determining regions (CDRs) and framework regions (FRs).

The CDRs include CDR1, CDR2, and CDR3, and the FRs include FR1, FR2, FR3, and FR4.

The CDR1 herein comprises the following formula (I):

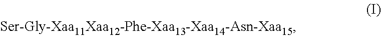

The CDR2 herein comprises the following formula (II):

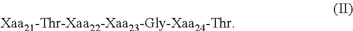

The CDR3 herein comprises the following formula (III):

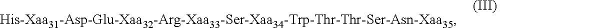

Wherein, Xaa₁₁ is independently selected from Arg, Ser, Thr; and/or Xaa₁₂ is independently selected from Ala, Gly, His, Ile, Met, Asn, Arg, Ser, Thr; and/or Xaa₁₃ is independently selected from Ile, Arg, Ser, Thr, Val; and/or Xaa₁₄ is independently selected from Ala, Ile, Leu; and/or Xaa₁₅ is independently selected from Ala, Ile, Leu, Ser, Thr, Val;

and/or Xaa₂₁ is independently selected from Phe, Ile, Leu, Val; and/or Xaa₂₂ is independently selected from Pro, Arg, Ser; and/or Xaa₂₃ is independently selected from Ala, Asp, Gly; and/or Xaa₂₄ is independently selected from Asp, Gly, Asn, Ser, Thr, Val;

and/or Xaa₃₁ is independently selected from Ile, Leu, Val; and/or Xaa₃₂ is independently selected from Ile, Leu, Val; and/or Xaa₃₃ is independently selected from Asp, Glu, Gln; and/or Xaa₃₄ is independently selected from Gly, Ile, Ser, Thr; and/or Xaa₃₅ is independently selected from Ile, Leu.

The amino acid substitution may generally be described as, wherein the amino acid residue may be substituted with an amino acid having a similar chemical structure, or may be substituted with an amino acid having a dissimilar chemical structure, so long as the function, activity or other biological characteristics of the polypeptide have little or no influence on the function, activity or other biological characteristics of the polypeptide. Preferably, the amino acid residue may be substituted with an amino acid having a similar chemical structure.

Examples of such substitution modes include, but are not limited to, those disclosed in documents WO 98/49185, WO 00/46383, and WO 01/09300. Additionally, the (preferred) types and/or combinations of such substitutions may be selected based on relevant information from other references cited in WO2018220235A1.

The amino acid substitutions of the present invention may include, but are not limited to, the following substitution groups: (a) Ala, Ser, Thr, Pro and Gly; (b) Asp, Asn, Glu and Gln; (c) His, Lys and Arg; (d) Met, Leu, Ile, Val and Cys; (e) Phe, Tyr and Trp.

Preferred amino acid substitutions may include, but are not limited to, the following modes: Ala is replaced with Gly or Ser; Arg is replaced with Lys; ASN is replaced with Gln or His; Asp is replaced with Glu; Cys is replaced with Ser or Thr; Gln is substituted into Asn; Glu is substituted into Asp; Gly is replaced with Ala or Pro; His is substituted into Asn or Gln; Ile is substituted into Leu or Val; Leu is substituted into Ile or Val; Met is substituted with Leu, Tyr or Leu; Phe is replaced with Met, Tyr or Leu; Ser is replaced with Thr; Thr is replaced with Ser; Tyr is replaced with Trp; Trp is substituted into Tyr.

For some preferred modes, in the above formula (I), Xaa₁₁ is Arg, Xaa₁₂ is Ser, Xaa₁₃ is Ser, Xaa₁₄ is Ile, and Xaa₁₅ is Thr (denoted as “CDR1-1”).

For some preferred modes, in the above formula (I), Xaa₁₁ is Ser, Xaa₁₂ is Arg, Xaa₁₃ is Ser, Xaa₁₄ is Ile, Xaa₁₅ is Thr (denoted as “CDR1-2”).

For some preferred modes, in the above formula (I), Xaa₁₁ is Arg, Xaa₁₂ is His, Xaa₁₃ is Ser, Xaa₁₄ is Ile, Xaa₁₅ is Thr (denoted as “CDR1-3”).

For some preferred modes, in the above formula (I), Xaa₁₁ is Arg, Xaa₁₂ is Ala, Xaa₁₃ is Ser, Xaa₁₄ is Ile, Xaa₁₅ is Thr (denoted as “CDR1-4”).

For some preferred modes, in the above formula (I), Xaa₁₁ is Ser, Xaa₁₂ is Ile, Xaa₁₃ is Ser, Xaa₁₄ is Ile, Xaa₁₅ is Ser (denoted as “CDR1-5”).

For some preferred modes, in the above formula (I), Xaa₁₁ is Ser, Xaa₁₂ is Arg, Xaa₁₃ is Ser, Xaa₁₄ is Ile, Xaa₁₅ is Ser (denoted as “CDR1-6”).

For some preferred modes, in the above formula (I), Xaa₁₁ is Ser, Xaa₁₂ is Ile, Xaa₁₃ is Ser, Xaa₁₄ is Ile, Xaa₁₅ is Leu (denoted as “CDR1-7”).

For some preferred modes, in the above formula (I), Xaa₁₁ is Ser, Xaa₁₂ is Thr, Xaa₁₃ is Ser, Xaa₁₄ is Ile, Xaa₁₅ is Thr (denoted as “CDR1-8”).

For some preferred modes, in the above formula (I), Xaa₁₁ is Ser, Xaa₁₂ is Gly, Xaa₁₃ is Ser, Xaa₁₄ is Ile, Xaa₁₅ is Thr (denoted as “CDR1-9”).

For some preferred modes, in the above formula (I), Xaa₁₁ is Ser, Xaa₁₂ is Ala, Xaa₁₃ is Ser, Xaa₁₄ is Ile, Xaa₁₅ is Thr (denoted as “CDR1-10”).

For some preferred modes, in the above formula (I), Xaa₁₁ is Ser, Xaa₁₂ is Asn, Xaa₁₃ is Arg, Xaa₁₄ is Ile, Xaa₁₅ is Thr (denoted as “CDR1-11”).

For some preferred modes, in the above formula (I), Xaa₁₁ is Ser, Xaa₁₂ is Asn, Xaa₁₃ is Ser, Xaa₁₄ is Ile, Xaa₁₅ is Thr (denoted as “CDR1-12”).

For some preferred modes, in the above formula (I), Xaa₁₁ is Ser, Xaa₁₂ is Ser, Xaa₁₃ is Thr, Xaa₁₄ is Ile, Xaa₁₅ is Thr (denoted as “CDR1-13”).

For some preferred modes, in the above formula (I), Xaa₁₁ is Ser, Xaa₁₂ is Ser, Xaa₁₃ is Ser, Xaa₁₄ is Ile, Xaa₁₅ is Thr (denoted as “CDR1-14”).

For some preferred modes, in the above formula (I), Xaa₁₁ is Arg, Xaa₁₂ is Gly, Xaa₁₃ is Ser, Xaa₁₄ is Ile, Xaa₁₅ is Thr (denoted as “CDR1-15”).

For some preferred modes, in the above formula (I), Xaa₁₁ is Ser, Xaa₁₂ is Ile, Xaa₁₃ is Ser, Xaa₁₄ is Ile, Xaa₁₅ is Thr (denoted as “CDR1-16”).

For some preferred modes, in the above formula (I), Xaa₁₁ is Ser, Xaa₁₂ is His, Xaa₁₃ is Ser, Xaa₁₄ is Ile, Xaa₁₅ is Thr (denoted as “CDR1-17”).

For some preferred modes, in the above formula (I), Xaa₁₁ is Arg, Xaa₁₂ is Ile, Xaa₁₃ is Ser, Xaa₁₄ is Ile, Xaa₁₅ is Thr (denoted as “CDR1-18”).

For some preferred modes, in the above formula (I), Xaa₁₁ is Ser, Xaa₁₂ is Met, Xaa₁₃ is Ser, Xaa₁₄ is Ile, Xaa₁₅ is Thr (denoted as “CDR1-19”).

For some preferred modes, in the above formula (I), Xaa₁₁ is Ser, Xaa₁₂ is Ser, Xaa₁₃ is Val, Xaa₁₄ is Ile, Xaa₁₅ is Thr (denoted as “CDR1-20”).

For some preferred modes, in the above formula (I), Xaa₁₁ is Ser, Xaa₁₂ is Ile, Xaa₁₃ is Ile, Xaa₁₄ is Ile, Xaa₁₅ is Thr (denoted as “CDR1-21”).

For some preferred modes, in the above formula (I), Xaa₁₁ is Ser, Xaa₁₂ is His, Xaa₁₃ is Ser, Xaa₁₄ is Leu, Xaa₁₅ is Thr (denoted as “CDR1-22”).

For some preferred modes, in the above formula (I), Xaa₁₁ is Ser, Xaa₁₂ is Thr, Xaa₁₃ is Ser, Xaa₁₄ is Leu, Xaa₁₅ is Thr (denoted as “CDR1-23”).

For some preferred modes, in the above formula (I), Xaa₁₁ is Ser, Xaa₁₂ is Arg, Xaa₁₃ is Ser, Xaa₁₄ is Leu, Xaa₁₅ is Thr (denoted as “CDR1-24”).

For some preferred modes, in the above formula (I), Xaa₁₁ is Ser, Xaa₁₂ is Ser, Xaa₁₃ is Ser, Xaa₁₄ is Ile, Xaa₁₅ is Ala (denoted as “CDR1-25”).

For some preferred modes, in the above formula (I), Xaa₁₁ is Arg, Xaa₁₂ is Arg, Xaa₁₃ is Thr, Xaa₁₄ is Ile, Xaa₁₅ is Thr (denoted as “CDR1-26”).

For some preferred modes, in the above formula (I), Xaa₁₁ is Arg, Xaa₁₂ is Asn, Xaa₁₃ is Ser, Xaa₁₄ is Leu, Xaa₁₅ is Thr (denoted as “CDR1-27”).

For some preferred modes, in the above formula (I), Xaa₁₁ is Arg, Xaa₁₂ is Ala, Xaa₁₃ is Ser, Xaa₁₄ is Leu, Xaa₁₅ is Thr (denoted as “CDR1-28”).

For some preferred modes, in the above formula (I), Xaa₁₁ is Ser, Xaa₁₂ is Ile, Xaa₁₃ is Thr, Xaa₁₄ is Ile, Xaa₁₅ is Ser (denoted as “CDR1-29”).

For some preferred modes, in the above formula (I), Xaa₁₁ is Ser, Xaa₁₂ is Met, Xaa₁₃ is Ser, Xaa₁₄ is Leu, Xaa₁₅ is Thr (denoted as “CDR1-30”).

For some preferred modes, in the above formula (I), Xaa₁₁ is Thr, Xaa₁₂ is Met, Xaa₁₃ is Ser, Xaa₁₄ is Leu, Xaa₁₅ is Thr (denoted as “CDR1-31”).

For some preferred modes, in the above formula (I), Xaa₁₁ is Thr, Xaa₁₂ is Arg, Xaa₁₃ is Ser, Xaa₁₄ is Ile, Xaa₁₅ is Thr (denoted as “CDR1-32”).

For some preferred modes, in the above formula (I), Xaa₁₁ is Ser, Xaa₁₂ is Arg, Xaa₁₃ is Ile, Xaa₁₄ is Ile, Xaa₁₅ is Thr (denoted as “CDR1-33”).

For some preferred modes, in the above formula (I), Xaa₁₁ is Ser, Xaa₁₂ is Ile, Xaa₁₃ is Val, Xaa₁₄ is Ile, Xaa₁₅ is Thr (denoted as “CDR1-34”).

For some preferred modes, in the above formula (I), Xaa₁₁ is Ser, Xaa₁₂ is Ser, Xaa₁₃ is Ser, Xaa₁₄ is Ala, Xaa₁₅ is Thr (denoted as “CDR1-35”).

For some preferred modes, in the above formula (I), Xaa₁₁ is Ser, Xaa₁₂ is Ile, Xaa₁₃ is Ser, Xaa₁₄ is Ala, Xaa₁₅ is Thr (denoted as “CDR1-36”).

For some preferred modes, in the above formula (I), Xaa₁₁ is Ser, Xaa₁₂ is Arg, Xaa₁₃ is Ser, Xaa₁₄ is Ile, Xaa₁₅ is Ile (denoted as “CDR1-37”).

For some preferred modes, in the above formula (I), Xaa₁₁ is Ser, Xaa₁₂ is His, Xaa₁₃ is Thr, Xaa₁₄ is Ile, Xaa₁₅ is Ser (denoted as “CDR1-38”).

For some preferred modes, in the above formula (I), Xaa₁₁ is Ser, Xaa₁₂ is Ile, Xaa₁₃ is Ser, Xaa₁₄ is Leu, Xaa₁₅ is Ala (denoted as “CDR1-39”).

For some preferred modes, in the above formula (I), Xaa₁₁ is Ser, Xaa₁₂ is Ile, Xaa₁₃ is Ser, Xaa₁₄ is Leu, Xaa₁₅ is Ser (denoted as “CDR1-40”).

For some preferred modes, in the above formula (I), Xaa₁₁ is Thr, Xaa₁₂ is Ile, Xaa₁₃ is Ser, Xaa₁₄ is Leu, Xaa₁₅ is Val (denoted as “CDR1-41”).

For some preferred modes, in the above formula (I), Xaa₁₁ is Thr, Xaa₁₂ is Asn, Xaa₁₃ is Ser, Xaa₁₄ is Ile, Xaa₁₅ is Val (denoted as “CDR1-42”).

For some preferred modes, in the above formula (II), Xaa₂₁ is Val, Xaa₂₂ is Arg, Xaa₂₃ is Gly, Xaa₂₄ is Ser (denoted as “CDR2-1”).

For some preferred modes, in the above formula (II), Xaa₂₁ is Val, Xaa₂₂ is Arg, Xaa₂₃ is Gly, Xaa₂₄ is Gly (denoted as “CDR2-2”).

For some preferred modes, in the above formula (II), Xaa₂₁ is Ile, Xaa₂₂ is Arg, Xaa₂₃ is Gly, Xaa₂₄ is Asn (denoted as “CDR2-3”).

For some preferred modes, in the above formula (II), Xaa₂₁ is Ile, Xaa₂₂ is Arg, Xaa₂₃ is Gly, Xaa₂₄ is Ser (denoted as “CDR2-4”).

For some preferred modes, in the above formula (II), Xaa₂₁ is Val, Xaa₂₂ is Arg, Xaa₂₃ is Gly, Xaa₂₄ is Asn (denoted as “CDR2-5”).

For some preferred modes, in the above formula (II), Xaa₂₁ is Ile, Xaa₂₂ is Pro, Xaa₂₃ is Gly, Xaa₂₄ is Asn (denoted as “CDR2-6”).

For some preferred modes, in the above formula (II), Xaa₂₁ is Val, Xaa₂₂ is Arg, Xaa₂₃ is Gly, Xaa₂₄ is Val (denoted as “CDR2-7”).

For some preferred modes, in the above formula (II), Xaa₂₁ is Val, Xaa₂₂ is Arg, Xaa₂₃ is Asp, Xaa₂₄ is Ser (denoted as “CDR2-8”).

For some preferred modes, in the above formula (II), Xaa₂₁ is Val, Xaa₂₂ is Arg, Xaa₂₃ is Gly, Xaa₂₄ is Thr (denoted as “CDR2-9”).

For some preferred modes, in the above formula (II), Xaa₂₁ is Phe, Xaa₂₂ is Arg, Xaa₂₃ is Ala, Xaa₂₄ is Asn (denoted as “CDR2-10”).

For some preferred modes, in the above formula (II), Xaa₂₁ is Val, Xaa₂₂ is Arg, Xaa₂₃ is Gly, Xaa₂₄ is Asp (denoted as “CDR2-11”).

For some preferred modes, in the above formula (II), Xaa₂₁ is Leu, Xaa₂₂ is Arg, Xaa₂₃ is Gly, Xaa₂₄ is Ser (denoted as “CDR2-12”).

For some preferred modes, in the above formula (II), Xaa₂₁ is Val, Xaa₂₂ is Pro, Xaa₂₃ is Ala, Xaa₂₄ is Ser (denoted as “CDR2-13”).

For some preferred modes, in the above formula (II), Xaa₂₁ is Val, Xaa₂₂ is Ser, Xaa₂₃ is Ala, Xaa₂₄ is Ser (denoted as “CDR2-14”).

For some preferred modes, in the above formula (II), Xaa₂₁ is Val, Xaa₂₂ is Ser, Xaa₂₃ is Gly, Xaa₂₄ is Gly (denoted as “CDR2-15”).

For some preferred modes, in the above formula (II), Xaa₂₁ is Val, Xaa₂₂ is Arg, Xaa₂₃ is Asp, Xaa₂₄ is Gly (denoted as “CDR2-16”).

For some preferred modes, in the above formula (II), Xaa₂₁ is Ile, Xaa₂₂ is Arg, Xaa₂₃ is Gly, Xaa₂₄ is Gly (denoted as “CDR2-17”).

For some preferred modes, in the above formula (II), Xaa₂₁ is Val, Xaa₂₂ is Arg, Xaa₂₃ is Ala, Xaa₂₄ is Ser (denoted as “CDR2-18”).

For some preferred modes, in the above formula (II), Xaa₂₁ is Val, Xaa₂₂ is Pro, Xaa₂₃ is Gly, Xaa₂₄ is Ser (denoted as “CDR2-19”).

For some preferred modes, in the above formula (II), Xaa₂₁ is Phe, Xaa₂₂ is Arg, Xaa₂₃ is Gly, Xaa₂₄ is Asn (denoted as “CDR2-20”).

For some preferred modes, in the above formula (II), Xaa₂₁ is Ile, Xaa₂₂ is Ser, Xaa₂₃ is Gly, Xaa₂₄ is Ser (denoted as “CDR2-21”).

For some preferred modes, in the above formula (III), Xaa₃₁ is Ile, Xaa₃₂ is Val, Xaa₃₃ is Gln, Xaa₃₄ is Ser, Xaa₃₅ is Ile (denoted as “CDR3-1”).

For some preferred modes, in the above formula (III), Xaa₃₁ is Val, Xaa₃₂ is Val, Xaa₃₃ is Gln, Xaa₃₄ is Ser, Xaa₃₅ is Ile (denoted as “CDR3-2”).

For some preferred modes, in the above formula (III), Xaa₃₁ is Val, Xaa₃₂ is Val, Xaa₃₃ is Glu, Xaa₃₄ is Ser, Xaa₃₅ is Leu (denoted as “CDR3-3”).

For some preferred modes, in the above formula (III), Xaa₃₁ is Val, Xaa₃₂ is Val, Xaa₃₃ is Gln, Xaa₃₄ is Ser, Xaa₃₅ is Leu (denoted as “CDR3-4”).

For some preferred modes, in the above formula (III), Xaa₃₁ is Val, Xaa₃₂ is Val, Xaa₃₃ is Asp, Xaa₃₄ is Ser, Xaa₃₅ is Ile (denoted as “CDR3-5”).

For some preferred modes, in the above formula (III), Xaa₃₁ is Leu, Xaa₃₂ is Val, Xaa₃₃ is Glu, Xaa₃₄ is Ser, Xaa₃₅ is Leu (denoted as “CDR3-6”).

For some preferred modes, in the above formula (III), Xaa₃₁ is Val, Xaa₃₂ is Ile, Xaa₃₃ is Glu, Xaa₃₄ is Ser, Xaa₃₅ is Leu (denoted as “CDR3-7”).

For some preferred modes, in the above formula (III), Xaa₃₁ is Ile, Xaa₃₂ is Ile, Xaa₃₃ is Glu, Xaa₃₄ is Ser, Xaa₃₅ is Leu (denoted as “CDR3-8”).

For some preferred modes, in the above formula (III), Xaa₃₁ is Val, Xaa₃₂ is Ile, Xaa₃₃ is Gln, Xaa₃₄ is Ser, Xaa₃₅ is Leu (denoted as “CDR3-9”).

For some preferred modes, in the above formula (III), Xaa₃₁ is Leu, Xaa₃₂ is Val, Xaa₃₃ is Gln, Xaa₃₄ is Ser, Xaa₃₅ is Ile (denoted as “CDR3-10”).

For some preferred modes, in the above formula (III), Xaa₃₁ is Leu, Xaa₃₂ is Val, Xaa₃₃ is Gln, Xaa₃₄ is Ser, Xaa₃₅ is Leu (denoted as “CDR3-11”).

For some preferred modes, in the above formula (III), Xaa₃₁ is Ile, Xaa₃₂ is Val, Xaa₃₃ is Gln, Xaa₃₄ is Ser, Xaa₃₅ is Leu (denoted as “CDR3-12”).

For some preferred modes, in the above formula (III), Xaa₃₁ is Val, Xaa₃₂ is Leu, Xaa₃₃ is Gln, Xaa₃₄ is Ser, Xaa₃₅ is Leu (denoted as “CDR3-13”).

For some preferred modes, in the above formula (III), Xaa₃₁ is Leu, Xaa₃₂ is Val, Xaa₃₃ is Gln, Xaa₃₄ is Thr, Xaa₃₅ is Leu (denoted as “CDR3-14”).

For some preferred modes, in the above formula (III), Xaa₃₁ is Val, Xaa₃₂ is Val, Xaa₃₃ is Gln, Xaa₃₄ is Thr, Xaa₃₅ is Leu (denoted as “CDR3-15”).

For some preferred modes, in the above formula (III), Xaa₃₁ is Val, Xaa₃₂ is Val, Xaa₃₃ is Glu, Xaa₃₄ is Thr, Xaa₃₅ is Leu (denoted as “CDR3-16”).

For some preferred modes, in the above formula (III), Xaa₃₁ is Leu, Xaa₃₂ is Val, Xaa₃₃ is Asp, Xaa₃₄ is Ser, Xaa₃₅ is Leu (denoted as “CDR3-17”).

For some preferred modes, in the above formula (III), Xaa₃₁ is Val, Xaa₃₂ is Ile, Xaa₃₃ is Gln, Xaa₃₄ is Ser, Xaa₃₅ is Ile (denoted as “CDR3-18”).

For some preferred modes, in the above formula (III), Xaa₃₁ is Val, Xaa₃₂ is Leu, Xaa₃₃ is Asp, Xaa₃₄ is Ser, Xaa₃₅ is Leu (denoted as “CDR3-19”).

For some preferred modes, in the above formula (III), Xaa₃₁ is Val, Xaa₃₂ is Val, Xaa₃₃ is Asp, Xaa₃₄ is Ser, Xaa₃₅ is Leu (denoted as “CDR3-20”).

For some preferred modes, in the above formula (III), Xaa₃₁ is Val, Xaa₃₂ is Leu, Xaa₃₃ is Glu, Xaa₃₄ is Gly, Xaa₃₅ is Leu (denoted as “CDR3-21”).

For some preferred modes, in the above formula (III), Xaa₃₁ is Val, Xaa₃₂ is Leu, Xaa₃₃ is Glu, Xaa₃₄ is Ser, Xaa₃₅ is Leu (denoted as “CDR3-22”).

For some preferred modes, in the above formula (III), Xaa₃₁ is Val, Xaa₃₂ is Leu, Xaa₃₃ is Gln, Xaa₃₄ is Thr, Xaa₃₅ is Leu (denoted as “CDR3-23”).

For some preferred modes, in the above formula (III), Xaa₃₁ is Ile, Xaa₃₂ is Ile, Xaa₃₃ is Glu, Xaa₃₄ is Thr, Xaa₃₅ is Leu (denoted as “CDR3-24”).

For some preferred modes, in the above formula (III), Xaa₃₁ is Val, Xaa₃₂ is Val, Xaa₃₃ is Glu, Xaa₃₄ is Thr, Xaa₃₅ is Ile (denoted as “CDR3-25”).

For some preferred modes, in the above formula (III), Xaa₃₁ is Val, Xaa₃₂ is Ile, Xaa₃₃ is Glu, Xaa₃₄ is Thr, Xaa₃₅ is Leu (denoted as “CDR3-26”).

For some preferred modes, in the above formula (III), Xaa₃₁ is Val, Xaa₃₂ is Val, Xaa₃₃ is Glu, Xaa₃₄ is Ile, Xaa₃₅ is Leu (denoted as “CDR3-27”).

For some preferred modes, in the above formula (III), Xaa₃₁ is Leu, Xaa₃₂ is Val, Xaa₃₃ is Gln, Xaa₃₄ is Thr, Xaa₃₅ is Ile (denoted as “CDR3-28”).

For some preferred modes, in the above formula (III), Xaa₃₁ is Val, Xaa₃₂ is Leu, Xaa₃₃ is Asp, Xaa₃₄ is Thr, Xaa₃₅ is Leu (denoted as “CDR3-29”).

For some preferred modes, in the above formula (III), Xaa₃₁ is Leu, Xaa₃₂ is Val, Xaa₃₃ is Glu, Xaa₃₄ is Thr, Xaa₃₅ is Leu (denoted as “CDR3-30”).

For some preferred modes, in the above formula (III), Xaa₃₁ is Val, Xaa₃₂ is Val, Xaa₃₃ is Gln, Xaa₃₄ is Thr, Xaa₃₅ is Ile (denoted as “CDR3-31”).

For some preferred modes, in the above formula (III), Xaa₃₁ is Val, Xaa₃₂ is Ile, Xaa₃₃ is Gln, Xaa₃₄ is Thr, Xaa₃₅ is Leu (denoted as “CDR3-32”).

Besides three complementarity determining regions (CDR1-CDR3), the amino acid sequence and structure of the above-mentioned nanobody further include a framework region.

For the framework region, it is more conservative than the complementarity determining region. A person skilled in the art can reasonably screen the sequence structure of the framework region according to the actual use and function of the nanobody. As for the amino acid sequences of the framework regions, amino acid sequences with a sequence identity of at least 50% are preferred, more preferably at least 70%, and even more preferably at least 95%.

The contribution of the framework region to affinity is small, so that the amino acid substitution in the framework region generally does not affect the affinity of the nanobody, as long as the amino acid of the framework region can exist in a soluble form, the amino acid in the framework region is also applicable to the above amino acid substitution manner. In the present invention, the framework regions of A4-H1 and A4-003 have an amino acid sequence identity of 82%, and the framework regions of A4-H3 and A4-015 have an amino acid sequence identity of 85%, neither of which affects the affinity of the original sequences.

As a framework region, an example is a framework region FR1, a framework region FR2, a framework region FR3, and a framework region FR4, but it can be considered that this is not limited thereto.

As a specific example of the above framework region, the following may be listed:

The FR1 described above is the following formula (IV):

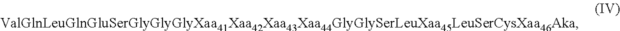

The FR2 described above is the following formula (V):

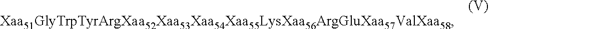

The FR3 described above is the following formula (VI):

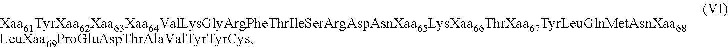

The FR4 described above is the following formula (VII):

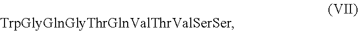

wherein,
Xaa₄₁ is independently selected from Leu or Ala; and/or Xaa₄₂ is independently selected from Val or Ala; and/or Xaa₄₃ is independently selected from Gln or His; and/or Xaa₄₄ is independently selected from Pro, Ala, Ser, or Thr; and/or Xaa₄₅ is independently selected from Arg or Lys; and/or Xaa₄₆ is independently selected from Ala, Val, Leu, Ile, or Phe; and/or Xaa₅₁ is independently selected from Met or Val; and/or Xaa₅₂ is independently selected from Gln or Arg; and/or Xaa₅₃ is independently selected from Ala or Arg; and/or Xaa₅₄ is independently selected from Pro or Ala; and/or Xaa₅₅ is independently selected from Gly, Pro, Ala, or Arg; and/or Xaa₅₆ is independently selected from Gln or Trp; and/or Xaa₅₇ is independently selected from Leu, Phe, Lys, Ile, or Met; and/or Xaa₅₈ is independently selected from Thr, Ala, Ser, Pro, or Asn; and/or Xaa₆₁ is independently selected from Asn, Ser, Lys, Thr, or Trp; and/or Xaa₆₂ is independently selected from Ala or Gly; and/or Xaa₆₃ is independently selected from Gly, Asp, Glu, or Asn; and/or Xaa₆₄ is independently selected from Ser or Phe; and/or Xaa₆₅ is independently selected from Ala, Pro, Thr, Gly, or Asp; and/or Xaa₆₆ is independently selected from Asn, Asp, Ser, or Thr; and/or Xaa₆₇ is independently selected from Val, Ile, or Ala; and/or Xaa₆₈ is independently selected from Ser, Thr, or Asn; and/or Xaa₆₉ is independently selected from Arg or Gln.

Examples of the amino acid sequences of the above-described nanobodies include:
CDR1 is CDR1-1, CDR2 is CDR2-1, CDR3 is CDR3-1, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-001, SEQ ID No: 1); or,
CDR1 is CDR1-1, CDR2 is CDR2-1, CDR3 is CDR3-1, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-002, SEQ ID No: 2); or,
CDR1 is CDR1-1, CDR2 is CDR2-1, CDR3 is CDR3-1, Xaa₄₁ is Leu, Xaa₄₂ is Val, Xaa₄₃ is Gln, Xaa₄₄ is Pro, Xaa₄₅ is Arg, Xaa₄₆ is Val, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-003, SEQ ID No: 3); or,
CDR1 is CDR1-2, CDR2 is CDR2-1, CDR3 is CDR3-1, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-004, SEQ ID No: 4); or,
CDR1 is CDR1-3, CDR2 is CDR2-2, CDR3 is CDR3-1, Xaa₄₁ is Leu, Xaa₄₂ is Val, Xaa₄₃ is Gln, Xaa₄₄ is Ala, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-005, SEQ ID No: 5); or,
CDR1 is CDR1-3, CDR2 is CDR2-1, CDR3 is CDR3-1, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-006, SEQ ID No: 6); or,
CDR1 is CDR1-3, CDR2 is CDR2-1, CDR3 is CDR3-1, Xaa₄₁ is Leu, Xaa₄₂ is Val, Xaa₄₃ is Gln, Xaa₄₄ is Ala, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-007, SEQ ID No: 7); or,
CDR1 is CDR1-4, CDR2 is CDR2-1, CDR3 is CDR3-2, Xaa₄₁ is Leu, Xaa₄₂ is Val, Xaa₄₃ is Gln, Xaa₄₄ is Ala, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Pro, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Ile, Xaa₆₈ is Ser, Xaa₆₉ is Arg, (designated as A4-008, SEQ ID No: 8); or,
CDR1 is CDR1-2, CDR2 is CDR2-1, CDR3 is CDR3-2, Xaa₄₁ is Leu, Xaa₄₂ is Ala, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Phe, Xaa₅₈ is Ala, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Thr, Xaa₆₉ is Arg, (designated as A4-009, SEQ ID No: 9); or,
CDR1 is CDR1-5, CDR2 is CDR2-5, CDR3 is CDR3-3, Xaa₄₁ is Leu, Xaa₄₂ is Val, Xaa₄₃ is Gln, Xaa₄₄ is Ala, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-010, SEQ ID No: 10); or,
CDR1 is CDR1-5, CDR2 is CDR2-5, CDR3 is CDR3-3, Xaa₄₁ is Leu, Xaa₄₂ is Val, Xaa₄₃ is Gln, Xaa₄₄ is Ala, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-011, SEQ ID No: 11); or,
CDR1 is CDR1-5, CDR2 is CDR2-5, CDR3 is CDR3-3, Xaa₄₁ is Ala, Xaa₄₂ is Val, Xaa₄₃ is Gln, Xaa₄₄ is Ala, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-012, SEQ ID No: 12); or,
CDR1 is CDR1-6, CDR2 is CDR2-5, CDR3 is CDR3-3, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-013, SEQ ID No: 13); or,
CDR1 is CDR1-6, CDR2 is CDR2-5, CDR3 is CDR3-3, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-014, SEQ ID No: 14); or,
CDR1 is CDR1-7, CDR2 is CDR2-6, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Ala, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-015, SEQ ID No: 15); or,
CDR1 is CDR1-7, CDR2 is CDR2-6, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-016, SEQ ID No: 16); or,
CDR1 is CDR1-7, CDR2 is CDR2-3, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-017, SEQ ID No: 17); or,
CDR1 is CDR1-2, CDR2 is CDR2-5, CDR3 is CDR3-5, Xaa₄₁ is Leu, Xaa₄₂ is Val, Xaa₄₃ is Gln, Xaa₄₄ is Ala, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Phe, Xaa₅₈ is Thr, Xaa₆₁ is Asn, Xaa₆₂ is Ala, Xaa₆₃ is Glu, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-018, SEQ ID No: 18); or,
CDR1 is CDR1-8, CDR2 is CDR2-4, CDR3 is CDR3-2, Xaa₄₁ is Leu, Xaa₄₂ is Val, Xaa₄₃ is Gln, Xaa₄₄ is Ala, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-019, SEQ ID No: 19); or,
CDR1 is CDR1-8, CDR2 is CDR2-3, CDR3 is CDR3-4, Xaa₄₁ is Leu, Xaa₄₂ is Val, Xaa₄₃ is Gln, Xaa₄₄ is Ala, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-020, SEQ ID No: 20); or,
CDR1 is CDR1-8, CDR2 is CDR2-3, CDR3 is CDR3-2, Xaa₄₁ is Leu, Xaa₄₂ is Val, Xaa₄₃ is Gln, Xaa₄₄ is Ala, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-021, SEQ ID No: 21); or,
CDR1 is CDR1-9, CDR2 is CDR2-8, CDR3 is CDR3-6, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Phe, Xaa₆₅ is Ala, Xaa₆₆ is Asn, Xaa₆₇ is Val, Xaa₆₈ is Ser, Xaa₆₉ is Arg, (designated as A4-022, SEQ ID No: 22); or,
CDR1 is CDR1-10, CDR2 is CDR2-19, CDR3 is CDR3-6, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ser, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Ile, Xaa₆₈ is Ser, Xaa₆₉ is Arg, (designated as A4-023, SEQ ID No: 23); or,
CDR1 is CDR1-11, CDR2 is CDR2-1, CDR3 is CDR3-3, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅ is Leu, Xaa₅₈ is Thr, Xaa₆₁ is Asn, Xaa₆₂ is Ala, Xaa₆₃ is Asp, Xaa₆₄ is Ser, Xaa₆₅ is Pro, Xaa₆₆ is Asn, Xaa₆₇ is Val, Xaa₆₈ is Ser, Xaa₆₉ is Arg, (designated as A4-024, SEQ ID No: 24); or,
CDR1 is CDR1-11, CDR2 is CDR2-1, CDR3 is CDR3-3, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Pro, Xaa₆₆ is Asp, Xaa₆₇ is Val, Xaa₆₈ is Ser, Xaa₆₉ is Arg, (designated as A4-025, SEQ ID No: 25); or,
CDR1 is CDR1-11, CDR2 is CDR2-1, CDR3 is CDR3-3, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Pro, Xaa₅₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-026, SEQ ID No: 26); or,
CDR1 is CDR1-12, CDR2 is CDR2-12, CDR3 is CDR3-3, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-027, SEQ ID No: 27); or,
CDR1 is CDR1-12, CDR2 is CDR2-11, CDR3 is CDR3-7, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-028, SEQ ID No: 28); or,
CDR1 is CDR1-12, CDR2 is CDR2-1, CDR3 is CDR3-8, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Arg, Xaa₅₄ is Ala, Xaa₅₅ is Gly, Xaa₅₆ is Gln, Xaa₅₇ is Leu, Xaa₅₈ is Thr, Xaa₅₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₅ is Ser, Xaa₆₉ is Arg, (designated as A4-029, SEQ ID No: 29); or,
CDR1 is CDR1-13, CDR2 is CDR2-1, CDR3 is CDR3-9, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Ile, Xaa₆₈ is Ser, Xaa₆₉ is Arg, (designated as A4-030, SEQ ID No: 30); or,
CDR1 is CDR1-9, CDR2 is CDR2-14, CDR3 is CDR3-9, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-031, SEQ ID No: 31); or,
CDR1 is CDR1-14, CDR2 is CDR2-8, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Val, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asn, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-032, SEQ ID No: 32); or,
CDR1 is CDR1-14, CDR2 is CDR2-8, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Val, Xaa₅₁ is Met, Xaa₅₂ is Arg, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asn, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-033, SEQ ID No: 33); or,
CDR1 is CDR1-15, CDR2 is CDR2-9, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-034, SEQ ID No: 34); or,
CDR1 is CDR1-15, CDR2 is CDR2-9, CDR3 is CDR3-4, Xaa₄₁ is Leu, Xaa₄₂ is Val, Xaa₄₃ is Gln, Xaa₄₄ is Ala, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-035, SEQ ID No: 35); or,
CDR1 is CDR1-15, CDR2 is CDR2-9, CDR3 is CDR3-4, Xaa₄₁ is Leu, Xaa₄₂ is Ala, Xaa₄₃ is Gln, Xaa₄₄ is Ala, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-036, SEQ ID No: 36); or,
CDR1 is CDR1-16, CDR2 is CDR2-1, CDR3 is CDR3-10, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₅ is Ser, Xaa₆₉ is Arg, (designated as A4-037, SEQ ID No: 37); or,
CDR1 is CDR1-17, CDR2 is CDR2-1, CDR3 is CDR3-11, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asn, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-038, SEQ ID No: 38); or,
CDR1 is CDR1-9, CDR2 is CDR2-1, CDR3 is CDR3-12, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Trp, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-039, SEQ ID No: 39); or,
CDR1 is CDR1-18, CDR2 is CDR2-1, CDR3 is CDR3-9, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-040, SEQ ID No: 40); or,
CDR1 is CDR1-15, CDR2 is CDR2-1, CDR3 is CDR3-13, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asn, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-041, SEQ ID No: 41); or,
CDR1 is CDR1-4, CDR2 is CDR2-8, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-042, SEQ ID No: 42); or,
CDR1 is CDR1-4, CDR2 is CDR2-8, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-043, SEQ ID No: 43); or,
CDR1 is CDR1-14, CDR2 is CDR2-7, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₅₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-044, SEQ ID No: 44); or,
CDR1 is CDR1-14, CDR2 is CDR2-7, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-045, SEQ ID No: 45); or,
CDR1 is CDR1-2, CDR2 is CDR2-1, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-046, SEQ ID No: 46); or,
CDR1 is CDR1-2, CDR2 is CDR2-1, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-047, SEQ ID No: 47); or,
CDR1 is CDR1-19, CDR2 is CDR2-5, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-048, SEQ ID No: 48); or,
CDR1 is CDR1-19, CDR2 is CDR2-5, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-049, SEQ ID No: 49); or,
CDR1 is CDR1-20, CDR2 is CDR2-1, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-050, SEQ ID No: 50); or,
CDR1 is CDR1-20, CDR2 is CDR2-1, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Arg, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-051, SEQ ID No: 51); or,
CDR1 is CDR1-21, CDR2 is CDR2-1, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-052, SEQ ID No: 52); or,
CDR1 is CDR1-21, CDR2 is CDR2-1, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Arg, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₅₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₅ is Ser, Xaa₆₉ is Arg, (designated as A4-053, SEQ ID No: 53); or,
CDR1 is CDR1-10, CDR2 is CDR2-1, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-054, SEQ ID No: 54); or,
CDR1 is CDR1-16, CDR2 is CDR2-1, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-055, SEQ ID No: 55); or,
CDR1 is CDR1-10, CDR2 is CDR2-1, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Lys, Xaa₅₈ is Ala, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-056, SEQ ID No: 56); or,
CDR1 is CDR1-16, CDR2 is CDR2-1, CDR3 is CDR3-4, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Lys, Xaa₅₈ is Ala, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-057, SEQ ID No: 57); or,
CDR1 is CDR1-16, CDR2 is CDR2-8, CDR3 is CDR3-14, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Ala, Xaa₅₆ is Gln, Xaa₅₇ is Ile, Xaa₅₈ is Thr, Xaa₆₁ is Asn, Xaa₆₂ is Ala, Xaa₆₃ is Glu, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-058, SEQ ID No: 58); or,
CDR1 is CDR1-22, CDR2 is CDR2-1, CDR3 is CDR3-15, Xaa₄₁ is Ala, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Arg, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Ile, Xaa₆₈ is Ser, Xaa₆₉ is Arg, (designated as A4-059, SEQ ID No: 59); or,
CDR1 is CDR1-23, CDR2 is CDR2-1, CDR3 is CDR3-15, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Ile, Xaa₅₈ is Thr, Xaa₆₁ is Asn, Xaa₆₂ is Ala, Xaa₆₃ is Glu, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-060, SEQ ID No: 60); or,
CDR1 is CDR1-24, CDR2 is CDR2-1, CDR3 is CDR3-15, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asn, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-061, SEQ ID No: 61); or,
CDR1 is CDR1-25, CDR2 is CDR2-9, CDR3 is CDR3-6, Xaa₄₁ is Leu, Xaa₄₂ is Val, Xaa₄₃ is Gln, Xaa₄₄ is Ala, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Pro, Xaa₆₆ is Asn, Xaa₆₇ is Val, Xaa₆₈ is Ser, Xaa₆₉ is Arg, (designated as A4-062, SEQ ID No: 62); or,
CDR1 is CDR1-26, CDR2 is CDR2-4, CDR3 is CDR3-12, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Arg, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Pro, Xaa₆₆ is Asn, Xaa₆₇ is Val, Xaa₆₈ is Ser, Xaa₆₉ is Arg, (designated as A4-063, SEQ ID No: 63); or,
CDR1 is CDR1-27, CDR2 is CDR2-15, CDR3 is CDR3-16, Xaa₄₁ is Leu, Xaa₄₂ is Val, Xaa₄₃ is Gln, Xaa₄₄ is Ser, Xaa₄₅ is Arg, Xaa₄₆ is Ala, Xaa₅₁ is Val, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-064, SEQ ID No: 64); or,
CDR1 is CDR1-27, CDR2 is CDR2-1, CDR3 is CDR3-3, Xaa₄₁ is Leu, Xaa₄₂ is Val, Xaa₄₃ is Gln, Xaa₄₄ is Ser, Xaa₄₅ is Arg, Xaa₄₆ is Ala, Xaa₅₁ is Val, Xaa₅₂ is Gln, Xaa₅₃ is Ala, Xaa₅₄ is Pro, Xaa₅₅ is Gly, Xaa₅₆ is Gln, Xaa₅ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-065, SEQ ID No: 65); or,
CDR1 is CDR1-28, CDR2 is CDR2-1, CDR3 is CDR3-3, Xaa₄₁ is Leu, Xaa₄₂ is Val, Xaa₄₃ is Gln, Xaa₄₄ is Thr, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-066, SEQ ID No: 66); or,
CDR1 is CDR1-28, CDR2 is CDR2-1, CDR3 is CDR3-3, Xaa₄₁ is Leu, Xaa₄₂ is Val, Xaa₄₃ is Gln, Xaa₄₄ is Thr, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Trp, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Thr, Xaa₆₆ is Asn, Xaa₆₇ is Val, Xaa₆₈ is Asn, Xaa₆₉ is Arg, (designated as A4-067, SEQ ID No: 67); or,
CDR1 is CDR1-28, CDR2 is CDR2-1, CDR3 is CDR3-3, Xaa₄₁ is Leu, Xaa₄₂ is Val, Xaa₄₃ is Gln, Xaa₄₄ is Thr, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Trp, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Asn, Xaa₆₉ is Arg, (designated as A4-068, SEQ ID No: 68); or,
CDR1 is CDR1-29, CDR2 is CDR2-5, CDR3 is CDR3-17, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Trp, Xaa₅₇ is Leu, Xaa₅₈ is Thr, Xaa₆₁ is Asn, Xaa₆₂ is Ala, Xaa₆₃ is Asn, Xaa₆₄ is Ser, Xaa₆₅ is Ala, Xaa₆₆ is Asn, Xaa₆₇ is Val, Xaa₆₈ is Thr, Xaa₆₉ is Arg, (designated as A4-069, SEQ ID No: 69); or,
CDR1 is CDR1-5, CDR2 is CDR2-1, CDR3 is CDR3-17, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Trp, Xaa₅₇ is Leu, Xaa₅₈ is Thr, Xaa₆₁ is Asn, Xaa₆₂ is Ala, Xaa₆₃ is Asn, Xaa₆₄ is Ser, Xaa₆₅ is Ala, Xaa₆₆ is Asn, Xaa₆₇ is Val, Xaa₆₈ is Thr, Xaa₆₉ is Arg, (designated as A4-070, SEQ ID No: 70); or,
CDR1 is CDR1-8, CDR2 is CDR2-1, CDR3 is CDR3-11, Xaa₄₁ is Ala, Xaa₄₂ is Val, Xaa₄₃ is Gln, Xaa₄₄ is Ala, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅ is Leu, Xaa₅₈ is Thr, Xaa₆₁ is Asn, Xaa₆₂ is Gly, Xaa₆₃ is Asp, Xaa₆₄ is Phe, Xaa₆₅ is Ala, Xaa₆₆ is Asn, Xaa₆₇ is Val, Xaa₆₈ is Thr, Xaa₆₉ is Arg, (designated as A4-071, SEQ ID No: 71); or,
CDR1 is CDR1-20, CDR2 is CDR2-1, CDR3 is CDR3-13, Xaa₄₁ is Leu, Xaa₄₂ is Ala, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Phe, Xaa₅₈ is Thr, Xaa₆₁ is Asn, Xaa₆₂ is Gly, Xaa₆₃ is Glu, Xaa₆₄ is Phe, Xaa₆₅ is Ala, Xaa₆₆ is Asn, Xaa₆₇ is Val, Xaa₆₈ is Ser, Xaa₆₉ is Arg, (designated as A4-072, SEQ ID No: 72); or,
CDR1 is CDR1-12, CDR2 is CDR2-1, CDR3 is CDR3-18, Xaa₄₁ is Leu, Xaa₄₂ is Val, Xaa₄₃ is Gln, Xaa₄₄ is Pro, Xaa₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Met, Xaa₅₈ is Thr, Xaa₆₁ is Asn, Xaa₆₂ is Gly, Xaa₆₃ is Asp, Xaa₆₄ is Phe, Xaa₆₅ is Ala, Xaa₆₆ is Asn, Xaa₆₇ is Val, Xaa₆₈ is Ser, Xaa₆₉ is Arg, (designated as A4-073, SEQ ID No: 73); or,
CDR1 is CDR1-30, CDR2 is CDR2-17, CDR3 is CDR3-13, Xaa₄₁ is Leu, Xaa₄₂ is Val, Xaa₄₃ is Gln, Xaa₄₄ is Ser, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Ser, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-074, SEQ ID No: 74); or,
CDR1 is CDR1-30, CDR2 is CDR2-17, CDR3 is CDR3-13, Xaa₄₁ is Leu, Xaa₄₂ is Val, Xaa₄₃ is Gln, Xaa₄₄ is Ser, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-075, SEQ ID No: 75); or,
CDR1 is CDR1-30, CDR2 is CDR2-17, CDR3 is CDR3-13, Xaa₄₁ is Leu, Xaa₄₂ is Val, Xaa₄₃ is His, Xaa₄₄ is Ser, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Ser, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-076, SEQ ID No: 76); or,
CDR1 is CDR1-19, CDR2 is CDR2-4, CDR3 is CDR3-19, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Phe, Xaa₅₈ is Thr, Xaa₆₁ is Asn, Xaa₆₂ is Ala, Xaa₆₃ is Asp, Xaa₆₄ is Phe, Xaa₆₅ is Ala, Xaa₆₆ is Asn, Xaa₆₇ is Val, Xaa₆₈ is Ser, Xaa₆₉ is Arg, (designated as A4-077, SEQ ID No: 77); or,
CDR1 is CDR1-31, CDR2 is CDR2-1, CDR3 is CDR3-20, Xaa₄₁ is Leu, Xaa₄₂ is Val, Xaa₄₃ is Gln, Xaa₄₄ is Pro, Xaa₄₅ is Arg, Xaa₄₆ is Leu, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Phe, Xaa₆₅ is Ala, Xaa₆₆ is Asn, Xaa₆₇ is Val, Xaa₆₈ is Ser, Xaa₆₉ is Arg, (designated as A4-078, SEQ ID No: 78); or,
CDR1 is CDR1-32, CDR2 is CDR2-13, CDR3 is CDR3-19, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Asn, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-079, SEQ ID No: 79); or,
CDR1 is CDR1-24, CDR2 is CDR2-1, CDR3 is CDR3-21, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Phe, Xaa₆₅ is Ala, Xaa₆₆ is Asn, Xaa₆₇ is Val, Xaa₆₈ is Ser, Xaa₆₉ is Arg, (designated as A4-080, SEQ ID No: 80); or,
CDR1 is CDR1-24, CDR2 is CDR2-1, CDR3 is CDR3-21, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-081, SEQ ID No: 81); or,
CDR1 is CDR1-24, CDR2 is CDR2-1, CDR3 is CDR3-21, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Lys, Xaa₆₂ is Ala, Xaa₆₃ is Asp, Xaa₆₄ is Phe, Xaa₆₅ is Ala, Xaa₆₆ is Asn, Xaa₆₇ is Val, Xaa₆₈ is Ser, Xaa₆₉ is Arg, (designated as A4-082, SEQ ID No: 82); or,
CDR1 is CDR1-24, CDR2 is CDR2-5, CDR3 is CDR3-22, Xaa₄₁ is Leu, Xaa₄₂ is Val, Xaa₄₃ is Gln, Xaa₄₄ is Thr, Xaa₄₅ is Lys, Xaa₄₆ is Ala, Xaa₅₁ is Met, Xaa₅₂ is Gln, Xaa₅₃ is Ala, Xaa₅₄ is Pro, Xaa₅₅ is Arg, Xaa₅₆ is Gln, Xaa₅₇ is Phe, Xaa₅₈ is Ala, Xaa₆₁ is Asn, Xaa₆₂ is Gly, Xaa₆₃ is Asp, Xaa₆₄ is Phe, Xaa₆₅ is Ala, Xaa₆₆ is Asn, Xaa₆₇ is Val, Xaa₆₈ is Ser, Xaa₆₉ is Arg, (designated as A4-083, SEQ ID No: 83); or,
CDR1 is CDR1-24, CDR2 is CDR2-5, CDR3 is CDR3-22, Xaa₄₁ is Leu, Xaa₄₂ is Val, Xaa₄₃ is Gln, Xaa₄₄ is Pro, Xaa₄₅ is Lys, Xaa₄₆ is Ala, Xaa₅₁ is Met, Xaa₅₂ is Gln, Xaa₅₃ is Ala, Xaa₅₄ is Pro, Xaa₅₅ is Arg, Xaa₅₆ is Gln, Xaa₅₇ is Phe, Xaa₅₈ is Ala, Xaa₆₁ is Asn, Xaa₆₂ is Gly, Xaa₆₃ is Asp, Xaa₆₄ is Phe, Xaa₆₅ is Ala, Xaa₆₆ is Asn, Xaa₆₇ is Val, Xaa₆₈ is Ser, Xaa₆₉ is Arg, (designated as A4-084, SEQ ID No: 84); or,
CDR1 is CDR1-24, CDR2 is CDR2-5, CDR3 is CDR3-22, Xaa₄₁ is Leu, Xaa₄₂ is Val, Xaa₄₃ is Gln, Xaa₄₄ is Pro, Xaa₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Phe, Xaa₅₈ is Ala, Xaa₆₁ is Asn, Xaa₆₂ is Gly, Xaa₆₃ is Asp, Xaa₆₄ is Phe, Xaa₆₅ is Ala, Xaa₆₆ is Asn, Xaa₆₇ is Val, Xaa₆₈ is Ser, Xaa₆₉ is Arg, (designated as A4-085, SEQ ID No: 85); or,
CDR1 is CDR1-24, CDR2 is CDR2-5, CDR3 is CDR3-22, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Ala, Xaa₆₃ is Asp, Xaa₆₄ is Phe, Xaa₆₅ is Ala, Xaa₆₆ is Asn, Xaa₆₇ is Val, Xaa₆₈ is Ser, Xaa₆₉ is Arg, (designated as A4-086, SEQ ID No: 86); or,
CDR1 is CDR1-24, CDR2 is CDR2-5, CDR3 is CDR3-22, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Gly, Xaa₆₃ is Asp, Xaa₆₄ is Phe, Xaa₆₅ is Ala, Xaa₆₆ is Asn, Xaa₆₇ is Val, Xaa₆₇ is Ser, Xaa₆₉ is Arg, (designated as A4-087, SEQ ID No: 87); or,
CDR1 is CDR1-24, CDR2 is CDR2-5, CDR3 is CDR3-22, Xaa₄₁ is Leu, Xaa₄₂ is Val, Xaa₄₃ is Gln, Xaa₄₄ is Thr, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ala, Xaa₆₁ is Asn, Xaa₆₂ is Gly, Xaa₆₃ is Asp, Xaa₆₄ is Phe, Xaa₆₅ is Ala, Xaa₆₆ is Asn, Xaa₆₇ is Val, Xaa₆₈ is Ser, Xaa₆₉ is Arg, (designated as A4-088, SEQ ID No: 88); or,
CDR1 is CDR1-12, CDR2 is CDR2-10, CDR3 is CDR3-20, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Gly, Xaa₆₆ is Asn, Xaa₆₇ is Val, Xaa₆₈ is Ser, Xaa₆₉ is Arg, (designated as A4-089, SEQ ID No: 89); or,
CDR1 is CDR1-33, CDR2 is CDR2-20, CDR3 is CDR3-16, Xaa₄₁ is Leu, Xaa₄₂ is Val, Xaa₄₃ is Gln, Xaa₄₄ is Pro, Xaa₄₅ is Arg, Xaa₄₆ is Val, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Glu, Xaa₆₄ is Ser, Xaa₆₅ is Gly, Xaa₆₆ is Ser, Xaa₆₇ is Val, Xaa₆₈ is Ser, Xaa₆₉ is Arg, (designated as A4-090, SEQ ID No: 90); or,
CDR1 is CDR1-2, CDR2 is CDR2-8, CDR3 is CDR3-23, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Arg, Xaa₅₄ is Ala, Xaa₅₅ is Gly, Xaa₅₆ is Gln, Xaa₅₇ is Leu, Xaa₅₈ is Thr, Xaa₆₁ is Thr, Xaa₆₂ is Ala, Xaa₆₃ is Asn, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-091, SEQ ID No: 91); or,
CDR1 is CDR1-16, CDR2 is CDR2-18, CDR3 is CDR3-7, Xaa₄₁ is Leu, Xaa₄₂ is Val, Xaa₄₃ is Gln, Xaa₄₄ is Thr, Xaa₄₅ is Arg, Xaa₄₆ is Leu, Xaa₅₁ is Met, Xaa₅₂ is Gln, Xaa₅₃ is Arg, Xaa₅₄ is Ala, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-092, SEQ ID No: 92); or,
CDR1 is CDR1-21, CDR2 is CDR2-1, CDR3 is CDR3-13, Xaa₄₁ is Leu, Xaa₄₂ is Val, Xaa₄₃ is Gln, Xaa₄₄ is Thr, Xaa₄₅ is Arg, Xaa₄₆ is Ala, Xaa₅₁ is Met, Xaa₅₂ is Gln, Xaa₅₃ is Arg, Xaa₅₄ is Ala, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Thr, Xaa₆₉ is Arg, (designated as A4-093, SEQ ID No: 93); or,
CDR1 is CDR1-34, CDR2 is CDR2-16, CDR3 is CDR3-8, Xaa₄₁ is Leu, Xaa₄₂ is Val, Xaa₄₃ is Gln, Xaa₄₄ is Pro, Xaa₄₅ is Arg, Xaa₄₆ is Val, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-094, SEQ ID No: 94); or,
CDR1 is CDR1-35, CDR2 is CDR2-3, CDR3 is CDR3-24, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Gly, Xaa₆₃ is Asp, Xaa₆₄ is Ser, Xaa₆₅ is Ala, Xaa₆₆ is Asp, Xaa₆₇ is Val, Xaa₆₈ is Ser, Xaa₆₉ is Arg, (designated as A4-095, SEQ ID No: 95); or,
CDR1 is CDR1-35, CDR2 is CDR2-3, CDR3 is CDR3-24, Xaa₄₁ is Leu, Xaa₄₂ is Val, Xaa₄₃ is Gln, Xaa₄₄ is Pro, Xaa₄₅ is Arg, Xaa₄₆ is Ile, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Gly, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-096, SEQ ID No: 96); or,
CDR1 is CDR1-36, CDR2 is CDR2-3, CDR3 is CDR3-7, Xaa₄₁ is Leu, Xaa₄₂ is Val, Xaa₄₃ is Gln, Xaa₄₄ is Pro, Xaa₄₅ is Arg, Xaa₄₆ is Ile, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Gly, Xaa₆₃ is Asp, Xaa₆₄ is Ser, Xaa₆₅ is Ala, Xaa₆₆ is Asn, Xaa₆₇ is Val, Xaa₆₈ is Thr, Xaa₆₉ is Arg, (designated as A4-097, SEQ ID No: 97); or,
CDR1 is CDR1-16, CDR2 is CDR2-3, CDR3 is CDR3-7, Xaa₄₁ is Leu, Xaa₄₂ is Val, Xaa₄₃ is Gln, Xaa₄₄ is Pro, Xaa₄₅ is Arg, Xaa₄₆ is Ile, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Gly, Xaa₆₃ is Asp, Xaa₆₄ is Ser, Xaa₆₅ is Ala, Xaa₆₆ is Asn, Xaa₆₇ is Val, Xaa₆₈ is Thr, Xaa₆₉ is Arg, (designated as A4-098, SEQ ID No: 98); or,
CDR1 is CDR1-14, CDR2 is CDR2-11, CDR3 is CDR3-8, Xaa₄₁ is Leu, Xaa₄₂ is Val, Xaa₄₃ is Gln, Xaa₄₄ is Thr, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Trp, Xaa₅₇ is Leu, Xaa₅₈ is Ala, Xaa₆₁ is Asn, Xaa₆₂ is Ala, Xaa₆₃ is Glu, Xaa₆₄ is Ser, Xaa₆₅ is Gly, Xaa₆₆ is Asn, Xaa₆₇ is Val, Xaa₆₈ is Ser, Xaa₆₉ is Arg, (designated as A4-099, SEQ ID No: 99); or,
CDR1 is CDR1-34, CDR2 is CDR2-12, CDR3 is CDR3-25, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ser, Xaa₆₁ is Asn, Xaa₆₂ is Ala, Xaa₆₃ is Asp, Xaa₆₄ is Ser, Xaa₆₅ is Asp, Xaa₆₆ is Asn, Xaa₆₇ is Ile, Xaa₆₈ is Ser, Xaa₆₉ is Arg, (designated as A4-100, SEQ ID No: 100); or,
CDR1 is CDR1-37, CDR2 is CDR2-9, CDR3 is CDR3-26, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ser, Xaa₆₁ is Asn, Xaa₆₂ is Ala, Xaa₆₃ is Asp, Xaa₆₄ is Ser, Xaa₆₅ is Pro, Xaa₆₆ is Asn, Xaa₆₇ is Ile, Xaa₆₈ is Ser, Xaa₆₉ is Arg, (designated as A4-101, SEQ ID No: 101); or,
CDR1 is CDR1-38, CDR2 is CDR2-9, CDR3 is CDR3-19, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Val, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Pro, Xaa₅₉ is Asn, Xaa₆₂ is Ala, Xaa₆₃ is Asp, Xaa₆₄ is Phe, Xaa₆₅ is Ala, Xaa₆₆ is Asn, Xaa₆₇ is Val, Xaa₆₈ is Ser, Xaa₆₉ is Arg, (designated as A4-102, SEQ ID No: 102); or,
CDR1 is CDR1-39, CDR2 is CDR2-4, CDR3 is CDR3-27, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Val, Xaa₆₈ is Ser, Xaa₆₉ is Arg, (designated as A4-103, SEQ ID No: 103); or,
CDR1 is CDR1-40, CDR2 is CDR2-21, CDR3 is CDR3-3, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Ala, Xaa₆₆ is Asn, Xaa₆₇ is Val, Xaa₆₈ is Asn, Xaa₆₉ is Gln, (designated as A4-104, SEQ ID No: 104); or,
CDR1 is CDR1-39, CDR2 is CDR2-21, CDR3 is CDR3-27, Xaa₄₁ is Leu, Xaa₄₂ is Val, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Ala, Xaa₆₈ is Asn, Xaa₆₉ is Gln, (designated as A4-105, SEQ ID No: 105); or,
CDR1 is CDR1-39, CDR2 is CDR2-21, CDR3 is CDR3-27, Xaa₄₁ is Leu, Xaa₄₂ is Val, Xaa₄₃ is Gln, Xaa₄₄ is Pro, Xaa₄₅ is Arg, Xaa₄₆ is Ile, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Ala, Xaa₆₆ is Asn, Xaa₆₇ is Ala, Xaa₆₈ is Asn, Xaa₆₉ is Gln, (designated as A4-106, SEQ ID No: 106); or,
CDR1 is CDR1-9, CDR2 is CDR2-12, CDR3 is CDR3-2, Xaa₄₁ is Leu, Xaa₄₂ is Val, Xaa₄₃ is Gln, Xaa₄₄ is Pro, Xaa₄₅ is Arg, Xaa₄₆ is Ile, Xaa₅₁ is Val, Xaa₅₂ is Gln, Xaa₅₃ is Ala, Xaa₅₄ is Pro, Xaa₅₅ is Ala, Xaa₅₆ is Gln, Xaa₅₇ is Leu, Xaa₅₈ is Thr, Xaa₆₁ is Asn, Xaa₆₂ is Ala, Xaa₆₃ is Asp, Xaa₆₄ is Ser, Xaa₆₅ is Pro, Xaa₆₆ is Asn, Xaa₆₇ is Val, Xaa₆₈ is Ser, Xaa₆₉ is Arg, (designated as A4-107, SEQ ID No: 107); or,
CDR1 is CDR1-9, CDR2 is CDR2-1, CDR3 is CDR3-28, Xaa₄₁ is Leu, Xaa₄₂ is Ala, Xaa₄₃ is Gln, Xaa₄₄ is Pro, Xaa₄₅ is Arg, Xaa₄₆ is Leu, Xaa₅₁ is Met, Xaa₅₂ is Gln, Xaa₅₃ is Ala, Xaa₅₄ is Pro, Xaa₅₅ is Pro, Xaa₅₆ is Trp, Xaa₅₇ is Leu, Xaa₅₈ is Thr, Xaa₆₁ is Asn, Xaa₆₂ is Ala, Xaa₆₃ is Gly, Xaa₆₄ is Ser, Xaa₆₅ is Pro, Xaa₆₆ is Asn, Xaa₆₇ is Val, Xaa₆₈ is Ser, Xaa₆₉ is Arg, (designated as A4-108, SEQ ID No: 108); or,
CDR1 is CDR1-41, CDR2 is CDR2-5, CDR3 is CDR3-29, Xaa₄₁ is Leu, Xaa₄₂ is Val, Xaa₄₃ is Gln, Xaa₄₄ is Ala, Xaa₄₅ is Arg, Xaa₄₆ is Ala, Xaa₅₁ is Met, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Ser, Xaa₆₁ is Asn, Xaa₆₂ is Ala, Xaa₆₃ is Asp, Xaa₆₄ is Ser, Xaa₆₅ is Ala, Xaa₆₆ is Thr, Xaa₆₇ is Val, Xaa₆₈ is Ser, Xaa₆₉ is Arg, (designated as A4-109, SEQ ID No: 109); or,
CDR1 is CDR1-42, CDR2 is CDR2-4, CDR3 is CDR3-17, Xaa₄₁ is Leu, Xaa₄₂ is Val, Xaa₄₃ is Gln, Xaa₄₄ is Pro, Xaa₄₅ is Arg, Xaa₄₆ is Phe, Xaa₅₁ is Val, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Asn, Xaa₆₂ is Ala, Xaa₆₃ is Glu, Xaa₆₄ is Ser, Xaa₆₅ is Ala, Xaa₆₆ is Asn, Xaa₆₇ is Val, Xaa₆₈ is Asn, Xaa₆₉ is Arg, (designated as A4-110, SEQ ID No: 110); or,
CDR1 is CDR1-12, CDR2 is CDR2-4, CDR3 is CDR3-30, Xaa₄₁ is Leu, Xaa₄₂ is Ala, Xaa₄₃ is Gln, Xaa₄₄ is Pro, Xaa₄₅ is Arg, Xaa₄₆ is Ala, Xaa₅₁ is Met, Xaa₅₂ is Gln, Xaa₅₃ is Arg, Xaa₅₄ is Ala, Xaa₅₅ is Gly, Xaa₅₆ is Gln, Xaa₅₇ is Phe, Xaa₅₈ is Thr, Xaa₆₁ is Asn, Xaa₆₂ is Gly, Xaa₆₃ is Asp, Xaa₆₄ is Ser, Xaa₆₅ is Ala, Xaa₆₆ is Thr, Xaa₆₇ is Val, Xaa₆₈ is Ser, Xaa₆₉ is Arg, (designated as A4-111, SEQ ID No: 111); or,
CDR1 is CDR1-16, CDR2 is CDR2-18, CDR3 is CDR3-31, Xaa₄₁ is Leu, Xaa₄₂ is Val, Xaa₄₃ is Gln, Xaa₄₄ is Thr, Xaa₄₅ is Arg, Xaa₄₆ is Ile, Xaa₅₁ is Val, Xaa₅₂ is Gln, Xaa₅₃ is Ala, Xaa₅₄ is Pro, Xaa₅₅ is Pro, Xaa₅₆ is Gln, Xaa₅₇ is Leu, Xaa₅₈ is Thr, Xaa₆₁ is Trp, Xaa₆₂ is Ala, Xaa₆₃ is Gly, Xaa₆₄ is Ser, Xaa₆₅ is Ala, Xaa₆₆ is Asn, Xaa₆₇ is Ile, Xaa₆₈ is Thr, Xaa₆₉ is Arg, (designated as A4-112, SEQ ID No: 112); or,
CDR1 is CDR1-2, CDR2 is CDR2-1, CDR3 is CDR3-32, Xaa₄₁ is Leu, Xaa₄₂ is Val, Xaa₄₃ is Gln, Xaa₄₄ is Thr, Xaa₄₅ is Arg, Xaa₄₆ is Val, Xaa₅₁ is Val, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Trp, Xaa₆₂ is Ala, Xaa₆₃ is Gly, Xaa₆₄ is Ser, Xaa₆₅ is Thr, Xaa₆₆ is Asn, Xaa₆₇ is Ile, Xaa₆₈ is Ser, Xaa₆₉ is Arg, (designated as A4-113, SEQ ID No: 113); or,
CDR1 is CDR1-16, CDR2 is CDR2-18, CDR3 is CDR3-23, Xaa₄₁ is Leu, Xaa₄₂ is Val, Xaa₄₃ is Gln, Xaa₄₄ is Thr, Xaa₄₅ is Arg, Xaa₄₆ is Val, Xaa₅₁ is Val, Xaa₅₂ is Gln, Xaa₅₃ is Ala, Xaa₅₄ is Pro, Xaa₅₅ is Gly, Xaa₅₆ is Gln, Xaa₅₇ is Leu, Xaa₅₈ is Thr, Xaa₆₁ is Trp, Xaa₆₂ is Ala, Xaa₆₃ is Gly, Xaa₆₄ is Ser, Xaa₆₅ is Thr, Xaa₆₆ is Asn, Xaa₆₇ is Ile, Xaa₆₈ is Ser, Xaa₆₉ is Arg, (designated as A4-114, SEQ ID No: 114).

But is not limited to these examples, as long as the amino acid substitutions which have little or essentially no influence on the function, activity or other biological properties of the polypeptide.

The total number of amino acid residues in a VHH domain will usually be in the range of 110 to 120, often between 112 and 115, and most preferably 113. It should be noted that smaller and longer sequences may also be suitable for the purposes described herein.

The nanobodies of the present invention belong to a nanobody family, with the same total length of amino acid sequences. The framework regions (FRs) and complementarity determining regions (CDRs) have the same length and high sequence identity, exhibit similar structures, and possess substantially equivalent antigen-binding capability. The FRs and CDRs are arranged in an alternating order. Thus, the general structure or sequence of an immunoglobulin variable domain may be indicated as follows: FR1-CDR1-FR2-CDR2-FR3-CDR3-FR4.

As for the preparation methods of “nanobodies”, in their broadest sense, they are not limited to specific biological resources or specific preparation methods. For example, the nanobodies of the present invention can be obtained as follows: (1) by isolating the VHH domain of naturally occurring heavy chain antibodies; (2) by expressing the nucleotide sequence encoding the naturally occurring VHH domain; (3) by “humanizing” the naturally occurring VHH domain (as described below) or by expressing the nucleic acid encoding the humanized VHH domain; (4) by preparing proteins, polypeptides or other amino acid sequences using synthetic or semi-synthetic techniques; (5) by preparing nucleic acids encoding nanobodies using nucleic acid synthesis techniques, and then expressing the obtained nucleic acids; and/or (6) by any combination of the foregoing.

In addition, a variant based on the nanobodies of the present invention further includes nanobodies having an amino acid sequence corresponding to a naturally occurring VHH domain but humanized. Humanization refers to replacing one or more amino acid residues in the sequence of the naturally occurring VHH domain with one or more amino acid residues present at the corresponding positions in the VH domain of a conventional human 4-chain antibody.

As specific examples of humanized amino acid sequence of nanobodies, SEQ ID No: 115 (designated “A4-H1”), SEQ ID No: 116 (“A4-H2”), SEQ ID No: 117 (“A4-H3”), SEQ ID No: 118 (“A4-H4”), SEQ ID No: 119 (“A4-H5”), and SEQ ID No: 120 (“A4-H6”) can be enumerated, but the scope is not limited thereto.

Additionally, the present invention also relates to proteins or polypeptides comprising at least one VHH domain or at least one derivative thereof, with an example being SEQ ID No: 147.

According to a non-limiting embodiment of the present invention, the above-mentioned polypeptide essentially consists of a nanobody. “Essentially consists of” means that the amino acid sequence of the polypeptide of the present invention is exactly the same as or corresponds to the amino acid sequence of the nanobody, wherein a limited number of amino acid residues, such as 1 to 10 amino acid residues, and preferably 1 to 6 amino acid residues, such as 1, 2, 3, 4, 5 or 6 amino acid residues, are added to the amino terminal end (N-terminal) and/or carboxyl terminal end (C-terminal) of the nanobody or polypeptide.

The further amino acid residues may or may not change, alter or otherwise influence other (biological) properties of the polypeptide of the invention and may or may not add further functionality to the polypeptide of the invention. For example, such amino acid residues:
a) may form a purification tag, i.e., an amino acid sequence or residue that facilitates the purification of the nanobody, such as through affinity techniques directed against the sequence or residue. Some preferred but non-limiting examples of such residues include poly-histidine tags (His₆ or His₈), GST-tag, MBP-tag, Myc-tag, Strep-tag, Flag-tag, HA-tag, V5-tag, S-tag, and E-tag;
b) may form a solubility tag, i.e., a tag that enhances the solubility of the nanobody, such as SUMO;
c) may be a N-terminal amino acid residues, such as Met, Ala, Gln, or MetAlaGln, AlaGln, thereby enabling expression in heterologous host cells or organisms;
d) may be a C-terminal Cys residue, which can form covalent bonds with —SH groups on ligands or interact with gold (Au) surfaces;
e) may form a linker to provide linkage or spacing between the nanobody and other moieties, such as GlySer linkers, IgG hinge, IgA hinge, or other synthetic linkers;
f) may be one or more amino acid residues that provide functional groups and/or have been functionalized in a known manner. For example, as is known in the art, amino acid residues such as lysine or cysteine enable the attachment of polyethylene glycol (PEG) groups.

The polypeptides of the present invention may further comprise two or more of the aforementioned nanobodies, also referred to as multivalent polypeptides.

The bivalent polypeptide of the present invention comprises two nanobodies, optionally linked by a linker sequence; the trivalent polypeptide comprises three nanobodies, optionally linked by two linker sequences; and the tetravalent polypeptide comprises four nanobodies, optionally linked by three linker sequences.

In the multivalent polypeptides of the present invention, the two or more nanobodies may be the same or different. For example, the two or more nanobodies in the multivalent polypeptides of the present invention may: target the same antigen, i.e., the same epitope of the antigen or two or more different epitopes of the antigen; target different antigens; or any combination thereof.

For example, the bivalent polypeptides AA or AB of the present invention may comprise two identical nanobodies A, or two different nanobodies A and B, wherein at least one of nanobody A and B is selected from the nanobodies of the present invention; may comprise a first nanobody targeting a certain epitope of a first antigen and a second nanobody targeting the same or different epitope of the antigen; or may comprise a first nanobody targeting a first antigen and a second nanobody targeting a second antigen different from the first antigen. Specific examples of the amino acid sequences of bivalent polypeptides include, but are not limited to, SEQ ID No: 121 (designated as “A4-B1”) and SEQ ID No: 122 (designated as “A4-B2”). Furthermore, specific examples of humanized bivalent polypeptides include, but are not limited to, SEQ ID No: 123 (designated as “A4-B3”) and SEQ ID No: 124 (designated as “A4-B4”).

For example, the trivalent polypeptides AAA, AAB, and ABC of the present invention may comprise three identical nanobodies A, two identical nanobodies A and another nanobody B, or three different nanobodies A, B, and C, wherein at least one of nanobodies A, B, and C is selected from the nanobodies of the present invention, regardless of the order of the three; they may comprise nanobodies that are the same or different against the same antigen; they may comprise two identical or different nanobodies targeting the same or different epitopes of a first antigen and a third nanobody targeting a second antigen different from the first antigen; or they may comprise a first nanobody targeting a first antigen, a second nanobody targeting a second antigen different from the first antigen, and a third nanobody targeting a third antigen different from both the first and second antigens.

For example, the tetravalent polypeptides AAAA, AAAB, AABC, and ABCD (regardless of the order) of the present invention may comprise four identical nanobodies A, three identical nanobodies A and another nanobody B, two identical nanobodies A and two different nanobodies B and C, or four different nanobodies A, B, C, and D, wherein at least one of nanobodies A, B, C, and D is selected from the nanobodies of the present invention, regardless of the order of the four; they may comprise nanobodies that are the same or different against the same antigen; they may comprise two identical or different nanobodies targeting the same or different epitopes of a first antigen and a third nanobody targeting a second antigen different from the first antigen; they may comprise a first nanobody targeting a first antigen, a second nanobody targeting a second antigen different from the first antigen, and a third nanobody targeting a third antigen different from both the first and second antigens; or they may comprise a first nanobody targeting a first antigen, a second nanobody targeting a second antigen different from the first antigen, a third nanobody targeting a third antigen different from both the first and second antigens, and a fourth nanobody targeting a fourth antigen different from the first, second, and third antigens.

A polypeptide of the present invention comprising at least two nanobodies, wherein at least one nanobody is directed against a first antigen and at least one nanobody is directed against a second antigen different from the first antigen (or a second nanobody against a different epitope of the first antigen), is also referred to as a “multispecific” antibody. Thus, a bispecific antibody includes at least one nanobody against a first antigen and at least one other nanobody against a second antigen, while a trispecific antibody includes at least one nanobody against a first antigen, at least one other nanobody against a second antigen, and at least one other nanobody against a third antigen; and so on.

Specific examples of amino acid sequences of multivalent polypeptides include trivalent polypeptides with amino acid sequences of SEQ ID No: 125 (designated “A4-C1”), SEQ ID No: 126 (“A4-C2”), SEQ ID No: 127 (“A4-C3”), SEQ ID No: 128 (“A4-C4”), SEQ ID No: 129 (“A4-C5”), SEQ ID No: 130 (“A4-C6”), SEQ ID No: 131 (“A4-C7”), SEQ ID No: 132 (“A4-C8”), SEQ ID No: 133 (“A4-C9”), and tetravalent polypeptides with amino acid sequences of SEQ ID No: 134 (“A4-D1”), SEQ ID No: 135 (“A4-D2”), SEQ ID No: 136 (“A4-D3”), SEQ ID No: 137 (“A4-D4”), SEQ ID No: 138 (“A4-D5”), SEQ ID No: 139 (“A4-D6”), SEQ ID No: 140 (“A4-D7”), SEQ ID No: 141 (“A4-D8”), SEQ ID No: 142 (“A4-D9”), SEQ ID No: 143 (“A4-D10”), SEQ ID No: 144 (“A4-D11”), SEQ ID No: 145 (“A4-D12”), SEQ ID No: 146 (“A4-D13”), but are not limited thereto.

Regarding the multivalent and multispecific polypeptides comprising one or more VHH domains and their preparation, reference may be made to the disclosure in EP 0822 985.

The linkers used for multivalent and multispecific polypeptides are well-known to those skilled in the art, including Gly-Ser linkers such as (Gly₄Ser)₃ or (Gly₃Ser₂)₃ as described in WO 99/42077, or hinge regions of naturally occurring heavy chain antibodies or their partial regions. For other suitable linkers, reference may also be made to the comprehensive background art cited above.

In addition to the aforementioned one or more nanobodies, the polypeptides of the present invention may further contain functional groups, moieties, or residues, such as therapeutic active substances, and/or tags, including fluorescent labels, isotope labels, biotin labels, and enzyme catalytic labels.

The dissociation equilibrium constant (K_{D})) for the binding of the nanobodies or polypeptides of the present invention to AAV is in the range of 10⁻⁶ to 10⁻¹¹ mol/L (M), preferably 10⁻⁷ to 10⁻¹¹ mol/L (M), and more preferably 10⁻⁸ to 10⁻¹⁰ mol/L (M).

The specific binding between the above-mentioned antigen and antigen-binding molecules can be determined by any suitable known method, including Scatchard analysis and/or competitive binding assays such as radioimmunoassay (RIA) and enzyme-linked immunosorbent assay (ELISA), as well as other novel methods known in the art, such as surface plasmon resonance (SPR) and/or biolayer interferometry (BLI) techniques.

The nanobodies, polypeptides, and corresponding encoding nucleic acids of the present invention can be synthesized using established methodologies, the specifics of which will be readily apparent to those skilled in the art from the subsequent detailed descriptions. A particularly efficacious approach for preparing these biomolecules generally encompasses the following sequential steps:
(1) Transfecting a suitable host cell or organism, or an alternative expression system, with a nucleic acid construct encoding the nanobody or polypeptide of interest, followed optionally by:
(2) Isolating and purifying the expressed nanobody or polypeptide using appropriate chromatographic or biochemical techniques.

Alternatively, an alternative protocol may involve:
(3) Culturing and maintaining a recombinant host under optimized conditions to facilitate the expression and production of the target nanobody or polypeptide; optionally followed by:
(4) Isolating and purifying the biomolecule from the culture medium or cellular lysate using established purification methodologies.

The nucleic acids of the present invention may exist in either single-stranded or double-stranded DNA or RNA formats, with double-stranded DNA being the preferred embodiment. For instance, the nucleic acid sequences may comprise genomic DNA, complementary DNA (cDNA), or synthetic DNA (e.g., codon-optimized sequences tailored for enhanced expression in the intended host cell or organism).

The nucleic acids of the present invention can be synthesized or isolated using methodologies well-established in the art, leveraging the amino acid sequence information provided herein for the nanobodies or polypeptides of interest. Alternatively, they may be isolated from natural sources. For instance, site-directed mutagenesis can be applied to the nucleic acid sequences of naturally occurring VHH domains to generate analog-encoding nucleic acids of the present invention.

The nucleic acids of the present invention may also exist as part of a genetic construct, a concept familiar to those skilled in the art. Such constructs typically comprise at least one nucleic acid of the invention and may be formatted as vectors, including plasmids, YACs, viral vectors, or transposons. Notably, the vector can be an expression vector, designed to facilitate in vitro or in vivo expression (e.g., within a suitable host cell, organism, or expression system).

The nucleic acids and/or genetic constructs of the present invention can be used to transform host cells or organisms for the purpose of expressing and producing the nanobodies or polypeptides of the invention. Suitable host systems, well established in the art, encompass various fungal, prokaryotic, or eukaryotic cell types or organisms, including but not limited to: bacterial strains such as Escherichia coli and Bacillus subtilis; fungal cells like Trichoderma, Aspergillus, and other filamentous fungi; yeast cells including Saccharomyces and Pichia species; amphibian cells/cell lines such as Xenopus oocytes; insect-derived cells/cell lines like Lepidoptera Sf9/Sf21 cells and Drosophila Schneider/Kc cell lines; plant/plant cells such as tobacco plants; mammalian cells/cell lines derived from humans or other mammals, including CHO (Chinese hamster ovary) cells, BHK (baby hamster kidney) cells, HeLa cells, and COS cells; as well as all other hosts commonly used for expressing antibody fragments (including single-domain antibodies and ScFv fragments), as is well known in the art.

For production, the nanobodies and polypeptides of the present invention can be produced in the milk of transgenic mammals, such as rabbits, cows, goats, or sheep, and also in plants or plant parts, including but not limited to their leaves, flowers, fruits, roots, or seeds. As mentioned above, one advantage of applying nanobodies is that polypeptides based thereon can be expressed and prepared in prokaryotic systems, and suitable prokaryotic expression systems, vectors, host cells, etc., are well-known to those skilled in the art, as referenced in the literature cited above. It should be noted, however, that the present invention in its broadest sense is not limited to expression in bacterial systems. Preferably, in the present invention, the nanobodies or polypeptides are produced in bacterial cells, particularly in bacterial cells suitable for large-scale pharmaceutical production, as described above. When expressing the nanobodies or polypeptides of the present invention in cells for production, the nanobodies or polypeptides of the present invention can be produced intracellularly (e.g., in the cytoplasm or periplasmic space), then isolated from the host cells, and optionally further purified; or they can be produced extracellularly (i.e., secreted expression), then isolated from the culture medium, and optionally further purified.

Some preferred but non-limiting vectors used together with these host cells include: vectors for expression in mammalian cells, such as pMANneo (Clonetech), pUCTtag (ATCC37460), and pMCIneo (Stratagene); vectors for expression in bacterial cells, such as pET vectors (Novagen) and pQE vectors (Qiagen); vectors for expression in yeast or other fungal cells, such as pYES2 (Invitrogen) and Pichia expression vectors (Invitrogen); vectors for expression in insect cells, such as pBlueBacII (Invitrogen) and other baculovirus vectors; and others.

Methods for transforming the host organisms or cells of the present invention are well-established in the art.

Following transformation, screening and selection procedures may be employed to identify hosts that have stably integrated the nucleotide sequences or genetic constructs of interest. The resulting transformed host cells (which may be maintained as stable cell lines) or host organisms (which may propagate as stable mutant lines or strains) constitute additional embodiments of the present invention.

Subsequently, the amino acid sequences of the present invention may be isolated from the host cells/host organisms and/or their culture media using established protein isolation and purification methodologies, including but not limited to chromatographic and electrophoretic methods (e.g., preparative chromatography), differential precipitation techniques (e.g., ammonium sulfate precipitation), affinity-based strategies (e.g., utilizing cleavable fusion tags genetically linked to the target sequences), immunological purification methods (e.g., antibody-based immunoprecipitation).

The nanobodies or polypeptides of the present invention exhibit specific binding affinity to adeno-associated virus (AAV) antigens. Accordingly, a preferred but non-limiting application of the present invention is an AAV adsorbent, which comprises a carrier matrix functionalized with the nanobodies or polypeptides.

The aforementioned AAV encompasses serotypes AAV1 through AAV10, and the nanobodies of the present invention demonstrate broad-spectrum binding capacity across diverse AAV serotypes.

Suitable carrier matrices for the adsorbent include porous materials such as agarose gel microspheres, cellulose beads, magnetic beads, silica microspheres, activated carbon, or resin microspheres. Commercially available carriers for use in the adsorbent include, but are not limited to, agarose gel Sepharose CL-6B (GE Healthcare, US) and resin microspheres of the Nanomicro series (Suzhou Nanomicro Technology CO., LTD.).

When utilizing the aforementioned carriers, it is preferable to subject them to activation procedures. Exemplary activation methods include, but are not limited to, sequential steps of epoxy activation, followed by diaminopropyl imine (DADPA) activation, and concluding with iodoacetic acid activation.

The AAV adsorbent is prepared by coupling the nanobodies or polypeptides to the activated carrier. The coupling methodology is not particularly restricted; for instance, the adsorbent may be obtained by mixing a purified nanobody or polypeptide solution with the activated carrier, followed by centrifugation to separate the matrix, and final washing/filtration of the gel to remove unbound components.

The adsorbent of the present invention is characterized by its specific recognition of adeno-associated virus (AAV).

The nanobodies, polypeptides, or adsorbents thereof find utility in both the purification of AAV, wherein the adsorbent selectively captures AAV from complex biological matrices, and the preparation of detection kits for AAV, wherein the nanobodies or polypeptides serve as specific binding agents for AAV diagnosis or quantification.

### EXAMPLES

The following examples are provided to illustrate specific embodiments of the present invention; however, the scope of the present invention is not limited thereto. Any modifications or selections made without altering the intended technical effects of the invention fall within its protective scope.

### A. Screening of Nanobodies Specifically Binding to AAV

### Example 1: Construction of Anti-AAV Nanobody Library

The phage display library employed in the present invention is an immune library utilizing T7 phage as a vector. The construction procedure is outlined below:
(1) Immunization and Sample Collection: Alpacas (No. 1139 and 462) were immunized with AAV. Following four immunization cycles, jugular venous blood was collected from both animals. Peripheral blood lymphocytes were isolated, and total RNA was extracted using the PuerLink™ RNA Mini Kit (Life Technologies, Cat. No. 12183018A).
(2) Reverse Transcription and Nested PCR Amplification: Total RNA was reverse-transcribed into cDNA, and the VHH gene was amplified via two rounds of nested PCR:

First Round PCR: Using cDNA as a template, UP primer1 and DOWN primer1 were employed as upstream and downstream primers, respectively. The amplified 650-750 bp fragment was gel-extracted and used as the template for the second round.

Second Round PCR: UP primer2 and DOWN primer2 were used as primers, yielding a 450-500 bp PCR product, which was subsequently purified.

UP primer1:
(SEQ ID No: 152)
CTTGGTGGTCCTGGCTGCTCT,
DOWN primer1:
(SEQ ID No: 153)
GGTACGTGCTGTTGAACTGTTCC,
UP primer2:
(SEQ ID No: 154)
TATCTAGTCGAATTCCGCCCAGGTGCAGCTC,
DOWN primer2:
(SEQ ID No: 155)
AGCGACTAAGCTT TTGTGGTTTTGGTGTC;

(3) Double Digestion and Fragment Recovery: The PCR product was double-digested with EcoRI and HindIII, separated by agarose gel electrophoresis, and a 350-500 bp gene fragment was recovered, corresponding to the VHH gene.
(4) Vector Ligation: The T7 vector (T7Select®10-3 Cloning Kit, Merck Millipore Novagen®, Cat. No. 70550-3) was ligated to the VHH gene fragment using T4 DNA ligase.
(5) Phage Library Construction: The ligation product was mixed with packaging proteins to reconstitute intact T7 phages, which were amplified to generate the primary phage library.
(6) Library Characterization: Titer determination of the primary library revealed a concentration of 3.14×10¹⁰ plaque-forming units per milliliter (pfu/mL), with a diversity of 1.7×10⁶ unique clones.

### Example 2: Nanobody Screening

### 1. Antigen Coating

AAV2 Coating: The AAV2 antigen was diluted to 10 μg/mL in TBS, and 100 μL of the solution was added to each well of a 96-well plate. The plate was incubated at 4° C. for 12 h.

Blocking: The antigen solution was discarded, and the plate was washed 3 times with TBS. After patting dry, 300 μL/well of 1% protein-free blocking solution (Sangon Biotech, Cat. No. C510042) was added, followed by 2 h incubation at room temperature. (Note: 1% protein-free blocking solution and 1% BSA were used alternately during screening.)

### 2. Phage Incubation and Elution

Phage Binding: The blocking solution was discarded, and the plate was washed 6 times with TBST. After patting dry, 100 μL/well of amplified phages was added, followed by 30 min incubation at room temperature.

Elution: The plate was washed 10 times with TBST, and T7 elution buffer (1% SDS) was added to elute the phages. Following 30 min incubation at room temperature, the eluate was amplified for subsequent screening rounds.

### 3. Multiserotype Panning

The above procedures were repeated for AAV5, AAV8, and AAV9 antigens:
Antigen Coating: Each antigen was diluted and coated using the same method as AAV2.
Sequential Panning: The eluate from the previous round was incubated with the new antigen for panning. The eluted phages were amplified and used for subsequent screening cycles.

### Example 3: Construction of Genetically Engineered Bacteria

(1) Following four rounds of screening, the eluted phages were amplified on solid medium. Single plaques were picked, and the plaque amplification solution was used as a template for PCR amplification. UP primer3 and DOWN primer3 were employed as the upstream and downstream primers, respectively.

UP primer3:
(SEQ ID No: 156)
TTCCTTAACATATGGCCCAGGTGCAGCTCGT,
DOWN primer3:
(SEQ ID No: 157)
TTAAGGAACTCGAGCACGGTGACCAGGGTC;

(2) A portion of the PCR products was subjected to outsourced sequencing, yielding the nanobody sequence information detailed in Table 1.
(3) The remaining PCR products were double-digested with NdeI and XhoI, and the digested fragments were gel-purified. The expression vector was digested using the same restriction enzymes, and the purified vector fragments were ligated to the target fragments with T4 DNA ligase. The ligation products were transformed into E. coli cells, resulting in genetically engineered bacteria capable of expressing AAV-specific nanobodies.

TABLE 1
Sequence Information of Nanobodies

| Sequence Name | Sequence Number |
|---|---|
| A4-001 | SEQ ID No: 1 |
| A4-002 | SEQ ID No: 2 |
| A4-003 | SEQ ID No: 3 |
| A4-004 | SEQ ID No: 4 |
| A4-005 | SEQ ID No: 5 |
| A4-006 | SEQ ID No: 6 |
| A4-007 | SEQ ID No: 7 |
| A4-008 | SEQ ID No: 8 |
| A4-009 | SEQ ID No: 9 |
| A4-010 | SEQ ID No: 10 |
| A4-011 | SEQ ID No: 11 |
| A4-012 | SEQ ID No: 12 |
| A4-013 | SEQ ID No: 13 |
| A4-014 | SEQ ID No: 14 |
| A4-015 | SEQ ID No: 15 |
| A4-016 | SEQ ID No: 16 |
| A4-017 | SEQ ID No: 17 |
| A4-018 | SEQ ID No: 18 |
| A4-019 | SEQ ID No: 19 |
| A4-020 | SEQ ID No: 20 |
| A4-021 | SEQ ID No: 21 |
| A4-022 | SEQ ID No: 22 |
| A4-023 | SEQ ID No: 23 |
| A4-024 | SEQ ID No: 24 |
| A4-025 | SEQ ID No: 25 |
| A4-026 | SEQ ID No: 26 |
| A4-027 | SEQ ID No: 27 |
| A4-028 | SEQ ID No: 28 |
| A4-029 | SEQ ID No: 29 |
| A4-030 | SEQ ID No: 30 |
| A4-031 | SEQ ID No: 31 |
| A4-032 | SEQ ID No: 32 |
| A4-033 | SEQ ID No: 33 |
| A4-034 | SEQ ID No: 34 |
| A4-035 | SEQ ID No: 35 |
| A4-036 | SEQ ID No: 36 |
| A4-037 | SEQ ID No: 37 |
| A4-038 | SEQ ID No: 38 |
| A4-039 | SEQ ID No: 39 |
| A4-040 | SEQ ID No: 40 |
| A4-041 | SEQ ID No: 41 |
| A4-042 | SEQ ID No: 42 |
| A4-043 | SEQ ID No: 43 |
| A4-044 | SEQ ID No: 44 |
| A4-045 | SEQ ID No: 45 |
| A4-046 | SEQ ID No: 46 |
| A4-047 | SEQ ID No: 47 |
| A4-048 | SEQ ID No: 48 |
| A4-049 | SEQ ID No: 49 |
| A4-050 | SEQ ID No: 50 |
| A4-051 | SEQ ID No: 51 |
| A4-052 | SEQ ID No: 52 |
| A4-053 | SEQ ID No: 53 |
| A4-054 | SEQ ID No: 54 |
| A4-055 | SEQ ID No: 55 |
| A4-056 | SEQ ID No: 56 |
| A4-057 | SEQ ID No: 57 |
| A4-058 | SEQ ID No: 58 |
| A4-059 | SEQ ID No: 59 |
| A4-060 | SEQ ID No: 60 |
| A4-061 | SEQ ID No: 61 |
| A4-062 | SEQ ID No: 62 |
| A4-063 | SEQ ID No: 63 |
| A4-064 | SEQ ID No: 64 |
| A4-065 | SEQ ID No: 65 |
| A4-066 | SEQ ID No: 66 |
| A4-067 | SEQ ID No: 67 |
| A4-068 | SEQ ID No: 68 |
| A4-069 | SEQ ID No: 69 |
| A4-070 | SEQ ID No: 70 |
| A4-071 | SEQ ID No: 71 |
| A4-072 | SEQ ID No: 72 |
| A4-073 | SEQ ID No: 73 |
| A4-074 | SEQ ID No: 74 |
| A4-075 | SEQ ID No: 75 |
| A4-076 | SEQ ID No: 76 |
| A4-077 | SEQ ID No: 77 |
| A4-078 | SEQ ID No: 78 |
| A4-079 | SEQ ID No: 79 |
| A4-080 | SEQ ID No: 80 |
| A4-081 | SEQ ID No: 81 |
| A4-082 | SEQ ID No: 82 |
| A4-083 | SEQ ID No: 83 |
| A4-084 | SEQ ID No: 84 |
| A4-085 | SEQ ID No: 85 |
| A4-086 | SEQ ID No: 86 |
| A4-087 | SEQ ID No: 87 |
| A4-088 | SEQ ID No: 88 |
| A4-089 | SEQ ID No: 89 |
| A4-090 | SEQ ID No: 90 |
| A4-091 | SEQ ID No: 91 |
| A4-092 | SEQ ID No: 92 |
| A4-093 | SEQ ID No: 93 |
| A4-094 | SEQ ID No: 94 |
| A4-095 | SEQ ID No: 95 |
| A4-096 | SEQ ID No: 96 |
| A4-097 | SEQ ID No: 97 |
| A4-098 | SEQ ID No: 98 |
| A4-099 | SEQ ID No: 99 |
| A4-100 | SEQ ID No: 100 |
| A4-101 | SEQ ID No: 101 |
| A4-102 | SEQ ID No: 102 |
| A4-103 | SEQ ID No: 103 |
| A4-104 | SEQ ID No: 104 |
| A4-105 | SEQ ID No: 105 |
| A4-106 | SEQ ID No: 106 |
| A4-107 | SEQ ID No: 107 |
| A4-108 | SEQ ID No: 108 |
| A4-109 | SEQ ID No: 109 |
| A4-110 | SEQ ID No: 110 |
| A4-111 | SEQ ID No: 111 |
| A4-112 | SEQ ID No: 112 |
| A4-113 | SEQ ID No: 113 |
| A4-114 | SEQ ID No: 114 |

### Example 4: Preparation of AAV Nanobodies

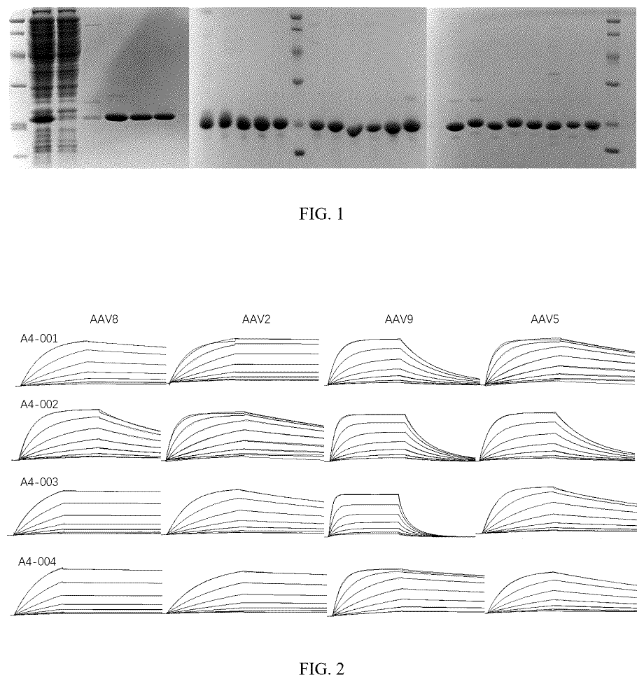

(1) Culture and Induction: The basal medium for nanobody expression was TB medium. Cultures were inoculated at a 5% inoculum ratio, incubated at 37° C. for 3-5 h, and induced overnight with 0.25 mM isopropyl-β-D-thiogalactopyranoside (IPTG) (the same induction conditions apply hereinafter).
(2) Cell Harvesting: Following induction, the culture was centrifuged at 4000 rpm for 20 min at 4° C. to harvest wet bacterial cells expressing nanobodies.
(3) Cell Lysis: Wet bacterial cells were resuspended in lysis buffer (10 mM imidazole, 500 mM NaCl, 0.02 M phosphate buffer (PB), pH 7.4) at a 1:10 (w/v) ratio, and cell disruption was performed using a 700 bar high-pressure homogenizer.
(4) Supernatant Collection: The cell lysate was centrifuged at 10,000 rpm for 20 min at 4° C., and the supernatant was collected.
(5) Affinity Chromatography Purification: The supernatant was filtered through a 0.45 μm membrane and subjected to purification using an affinity chromatography column (GE Healthcare, USA) packed with Ni Sepharose High Performance.
(6) Affinity-purified nanobodies were analyzed by SDS-PAGE for purity, and high-purity fractions were selected for protein concentration measurement via the BCA method. The SDS-PAGE results (FIG. 1) show correct band positions and high purity after single-step purification. Owing to their membership in the same family, the nanobodies exhibit similar molecular weights and charge properties, resulting in nearly identical electrophoresis band positions. For brevity, only the SDS-PAGE images of A4-001 (SEQ ID No: 1) to A4-022 (SEQ ID No: 22) are presented, with FIG. 1 described as follows:

Left panel (left-to-right): Protein marker, whole-cell lysate supernatant of A4-001 (SEQ ID No: 1), cell debris precipitate, purification flow-through, and purified A4-001 to A4-003 (SEQ ID Nos: 1-3).

Middle panel (left-to-right): Purified A4-004 to A4-008 (SEQ ID Nos: 4-8), protein marker, and purified A4-009 to A4-014 (SEQ ID Nos: 9-14).

Right panel (left-to-right): Purified A4-015 to A4-022 (SEQ ID Nos: 15-22) and protein marker.

### Example 5: Analysis of Nanobody Binding to AAV by SPR Technology

AAV was amine-coupled to a CM5 sensor chip at a density of 500-800 response units (RU). Nanobodies were injected at seven concentrations (1-100 nM) with a flow rate of 45 μL/min, and chip regeneration was performed using glycine-HCl (pH 1.5). Binding curves at different nanobody concentrations were used to calculate kinetic parameters (Kₐ, Kₐ, K_{D}) (Table 2 and FIG. 2). For conciseness, FIG. 2 shows kinetic sensorgrams of nanobodies A4-001 to A4-004 only. From top to bottom, the curves represent responses at 100 nM, 50 nM, 25 nM, 12.5 nM, 6.25 nM, 3.125 nM, and 1.5625 nM, with kinetic parameters derived by equation fitting (Table 2). The nanobodies exhibit high affinity for AAV2, AAV5, AAV8, and AAV9, with K_{D} values ranging from 10⁻⁶ to 10⁻¹¹ M.

TABLE 2
Affinity of Nanobodies to Different AAV Serotypes

| | Antigen | | | | | | | | | | | |
|---|---|---|---|---|---|---|---|---|---|---|---|---|
| Nanobody | AAV8 | | | AAV2 | | | AAV9 | | | AAV5 | | |
| Name | kₐ(1/MS) | k_{d}(1/S) | K_{D}(M) | kₐ(1/MS) | k_{d}(1/S) | K_{D}(M) | kₐ(1/MS) | k_{d}(1/S) | K_{D}(M) | kₐ(1/MS) | k_{d}(1/S) | K_{D}(M) |
| A4-001 | 1.84E+ | 6.69E− | 3.64E− | 6.01E+ | 6.35E− | 1.06E− | 8.16E+ | 1.16E− | 1.42E− | 1.51E+ | 9.80E− | 6.47E− |
| | 05 | 04 | 09 | 04 | 05 | 09 | 04 | 02 | 07 | 05 | 04 | 09 |
| A4-002 | 1.07E+ | 6.18E− | 5.77E− | 4.10E+ | 6.91E− | 1.69E− | 4.75E+ | 1.10E− | 2.32E− | 4.57E+ | 6.30E− | 1.38E− |
| | 05 | 03 | 08 | 04 | 04 | 08 | 04 | 02 | 07 | 03 | 04 | 07 |
| A4-003 | 2.32E+ | 2.77E− | 1.20E− | 1.80E+ | 1.09E− | 6.07E− | 8.24E+ | 1.52E− | 1.84E− | 2.31E+ | 1.90E− | 8.24E− |
| | 05 | 04 | 09 | 05 | 03 | 09 | 03 | 03 | 07 | 04 | 04 | 09 |
| A4-004 | 1.67E+ | 2.86E− | 1.71E− | 7.50E+ | 3.30E− | 4.40E− | 1.80E+ | 8.10E− | 4.51E− | 1.81E+ | 6.78E− | 3.75E− |
| | 05 | 04 | 09 | 03 | 04 | 08 | 05 | 04 | 09 | 05 | 03 | 08 |
| A4-005 | 4.49E+ | 1.97E− | 4.39E− | 1.42E+ | 1.80E− | 1.27E− | 2.37E+ | 2.10E− | 8.85E− | 1.04E+ | 1.93E− | 1.86E− |
| | 05 | 03 | 09 | 05 | 03 | 08 | 05 | 02 | 08 | 03 | 03 | 06 |
| A4-006 | 3.81E+ | 2.24E− | 5.87E− | 1.13E+ | 1.44E− | 1.27E− | 1.27E+ | 2.44E− | 1.92E− | 1.79E+ | 3.20E− | 1.78E− |
| | 05 | 03 | 09 | 05 | 03 | 08 | 05 | 02 | 07 | 05 | 04 | 09 |
| A4-007 | 7.73E+ | 3.67E− | 4.74E− | 1.69E+ | 1.70E− | 1.00E− | 1.38E+ | 8.90E− | 6.43E− | 1.97E+ | 2.48E− | 1.26E− |
| | 04 | 03 | 08 | 04 | 03 | 07 | 05 | 04 | 09 | 04 | 02 | 06 |
| A4-008 | 3.14E+ | 1.64E− | 5.23E− | 3.69E+ | 2.10E− | 5.70E− | 8.27E+ | 1.20E− | 1.45E− | 2.64E+ | 1.74E− | 6.60E− |
| | 03 | 03 | 07 | 03 | 04 | 08 | 03 | 04 | 08 | 05 | 02 | 08 |
| A4-009 | 2.14E+ | 2.19E− | 1.03E− | 5.41E+ | 1.11E− | 2.06E− | 1.39E+ | 1.39E− | 1.00E− | 4.97E+ | 6.98E− | 1.41E− |
| | 04 | 03 | 07 | 04 | 02 | 07 | 05 | 02 | 07 | 04 | 03 | 07 |
| A4-010 | 1.47E+ | 2.49E− | 1.69E− | 5.37E+ | 2.89E− | 5.38E− | 8.66E+ | 3.29E− | 3.80E− | 1.42E+ | 3.80E− | 2.67E− |
| | 04 | 03 | 07 | 04 | 02 | 07 | 04 | 02 | 07 | 03 | 04 | 07 |
| A4-011 | 8.94E+ | 2.72E− | 3.04E− | 1.63E+ | 4.62E− | 2.84E− | 4.39E+ | 5.82E− | 1.32E− | 9.46E+ | 2.80E− | 2.96E− |
| | 04 | 03 | 08 | 05 | 02 | 07 | 05 | 02 | 07 | 03 | 04 | 08 |
| A4-012 | 1.29E+ | 4.17E− | 3.24E− | 4.70E+ | 5.54E− | 1.18E− | 1.03E+ | 6.94E− | 6.72E− | 9.31E+ | 1.73E− | 1.86E− |
| | 04 | 03 | 07 | 04 | 02 | 06 | 05 | 02 | 07 | 04 | 03 | 08 |
| A4-013 | 6.06E+ | 7.37E− | 1.22E− | 2.25E+ | 5.43E− | 2.41E− | 7.91E+ | 3.79E− | 4.80E− | 1.52E+ | 1.10E− | 7.24E− |
| | 03 | 03 | 06 | 04 | 02 | 06 | 04 | 02 | 07 | 04 | 04 | 09 |
| A4-014 | 2.84E+ | 1.09E− | 3.84E− | 4.14E+ | 1.04E− | 2.51E− | 6.40E+ | 8.50E− | 1.33E− | 8.86E+ | 5.29E− | 5.97E− |
| | 03 | 03 | 07 | 03 | 03 | 07 | 04 | 04 | 08 | 03 | 03 | 07 |
| A4-015 | 1.74E+ | 1.70E− | 9.74E− | 3.72E+ | 6.90E− | 1.85E− | 5.01E+ | 1.70E− | 3.39E− | 1.89E+ | 5.20E− | 2.76E− |
| | 05 | 03 | 09 | 04 | 04 | 08 | 04 | 04 | 09 | 05 | 04 | 09 |
| A4-016 | 1.52E+ | 1.46E− | 9.58E− | 1.04E+ | 1.68E− | 1.61E− | 3.60E+ | 4.70E− | 1.31E− | 6.60E+ | 1.58E− | 2.39E− |
| | 05 | 03 | 09 | 05 | 03 | 08 | 03 | 04 | 07 | 04 | 03 | 08 |
| A4-017 | 1.43E+ | 2.00E− | 1.40E− | 2.18E+ | 3.40E− | 1.56E− | 5.53E+ | 4.00E− | 7.24E− | 3.03E+ | 1.64E− | 5.42E− |
| | 03 | 04 | 07 | 03 | 04 | 07 | 04 | 05 | 10 | 03 | 03 | 07 |
| A4-018 | 1.12E+ | 3.92E− | 3.49E− | 2.29E+ | 1.00E− | 4.36E− | 1.20E+ | 7.45E− | 6.21E− | 1.46E+ | 1.40E− | 9.62E− |
| | 05 | 03 | 08 | 04 | 04 | 09 | 05 | 03 | 08 | 05 | 04 | 10 |
| A4-019 | 1.13E+ | 7.70E− | 6.84E− | 9.96E+ | 6.60E− | 6.62E− | 1.17E+ | 5.25E− | 4.51E− | 3.19E+ | 1.56E− | 4.89E− |
| | 05 | 04 | 09 | 04 | 04 | 09 | 04 | 03 | 07 | 04 | 03 | 08 |
| A4-020 | 1.12E+ | 5.66E− | 5.06E− | 2.35E+ | 1.59E− | 6.77E− | 2.30E+ | 1.69E− | 7.37E− | 5.55E+ | 9.40E− | 1.69E− |
| | 05 | 05 | 10 | 04 | 02 | 07 | 04 | 03 | 08 | 03 | 04 | 07 |
| A4-021 | 2.09E+ | 1.55E− | 7.41E− | 5.48E+ | 4.37E− | 7.98E− | 7.47E+ | 1.40E− | 1.87E− | 1.20E+ | 9.40E− | 7.81E− |
| | 05 | 03 | 09 | 04 | 05 | 10 | 04 | 04 | 09 | 05 | 04 | 09 |
| A4-022 | 5.21E+ | 6.20E− | 1.19E− | 1.48E+ | 3.51E− | 2.37E− | 3.51E+ | 1.00E− | 2.85E− | 1.58E+ | 1.52E− | 9.61E− |
| | 04 | 05 | 09 | 04 | 04 | 08 | 03 | 04 | 08 | 05 | 03 | 09 |
| A4-023 | 8.73E+ | 1.25E− | 1.43E− | 5.95E+ | 1.93E− | 3.24E− | 1.95E+ | 5.60E− | 2.88E− | 1.02E+ | 1.26E− | 1.24E− |
| | 03 | 03 | 07 | 04 | 03 | 08 | 05 | 04 | 09 | 05 | 03 | 08 |
| A4-024 | 7.08E+ | 1.48E− | 2.09E− | 1.08E+ | 6.90E− | 6.39E− | 6.90E+ | 1.73E− | 2.51E− | 3.67E+ | 1.30E− | 3.55E− |
| | 03 | 03 | 07 | 03 | 04 | 07 | 03 | 03 | 07 | 03 | 04 | 08 |
| A4-025 | 1.24E+ | 2.60E− | 2.09E− | 1.49E+ | 4.20E− | 2.81E− | 1.63E+ | 1.20E− | 7.37E− | 4.80E+ | 4.10E− | 8.55E− |
| | 05 | 04 | 09 | 05 | 04 | 09 | 05 | 04 | 10 | 03 | 04 | 08 |
| A4-026 | 6.77E+ | 1.84E− | 2.72E− | 1.27E+ | 6.69E− | 5.28E− | 8.16E+ | 2.53E− | 3.10E− | 1.67E+ | 6.25E− | 3.74E− |
| | 04 | 03 | 08 | 05 | 03 | 08 | 04 | 03 | 08 | 05 | 03 | 08 |
| A4-027 | 8.10E+ | 8.50E− | 1.05E− | 6.17E+ | 4.50E− | 7.30E− | 4.09E+ | 8.50E− | 2.08E− | 8.22E+ | 1.94E− | 2.36E− |
| | 03 | 04 | 07 | 03 | 04 | 08 | 03 | 04 | 07 | 03 | 03 | 07 |
| A4-028 | 1.33E+ | 8.40E− | 6.34E− | 1.94E+ | 1.06E− | 5.47E− | 8.24E+ | 9.00E− | 1.09E− | 6.13E+ | 1.62E− | 2.64E− |
| | 05 | 04 | 09 | 04 | 03 | 08 | 04 | 05 | 09 | 04 | 03 | 08 |
| A4-029 | 8.23E+ | 4.00E− | 4.86E− | 4.49E+ | 1.47E− | 3.28E− | 9.69E+ | 1.41E− | 1.46E− | 1.67E+ | 1.55E− | 9.28E− |
| | 04 | 05 | 10 | 04 | 03 | 08 | 04 | 03 | 08 | 04 | 03 | 08 |
| A4-030 | 7.78E+ | 3.40E− | 4.37E− | 1.94E+ | 2.10E− | 1.08E− | 9.20E+ | 2.50E− | 2.72E− | 3.32E+ | 2.80E− | 8.44E− |
| | 03 | 04 | 08 | 03 | 04 | 07 | 03 | 04 | 08 | 03 | 04 | 08 |
| A4-031 | 5.07E+ | 9.23E− | 1.82E− | 1.06E+ | 3.23E− | 3.05E− | 3.69E+ | 8.24E− | 2.24E− | 1.49E+ | 6.24E− | 4.19E− |
| | 04 | 03 | 07 | 05 | 03 | 08 | 03 | 03 | 06 | 05 | 03 | 08 |
| A4-032 | 1.32E+ | 1.69E− | 1.28E− | 1.58E+ | 4.00E− | 2.53E− | 6.59E+ | 1.08E− | 1.64E− | 1.34E+ | 9.00E− | 6.70E− |
| | 05 | 03 | 08 | 05 | 04 | 09 | 04 | 03 | 08 | 05 | 04 | 09 |
| A4-033 | 3.81E+ | 3.40E− | 8.92E− | 6.75E+ | 1.86E− | 2.76E− | 2.27E+ | 4.50E− | 1.99E− | 8.22E+ | 2.00E− | 2.43E− |
| | 03 | 04 | 08 | 03 | 03 | 07 | 03 | 04 | 07 | 03 | 03 | 07 |
| A4-034 | 5.99E+ | 4.70E− | 7.84E− | 1.96E+ | 6.60E− | 3.36E− | 1.90E+ | 1.29E− | 6.80E− | 1.72E+ | 2.10E− | 1.22E− |
| | 04 | 04 | 09 | 05 | 04 | 09 | 05 | 03 | 09 | 04 | 04 | 08 |
| A4-035 | 1.30E+ | 1.70E− | 1.31E− | 7.53E+ | 4.10E− | 5.45E− | 5.78E+ | 1.00E− | 1.73E− | 1.99E+ | 5.00E− | 2.52E− |
| | 05 | 04 | 09 | 04 | 04 | 09 | 04 | 04 | 09 | 05 | 04 | 09 |
| A4-036 | 6.70E+ | 2.25E− | 3.36E− | 6.12E+ | 4.31E− | 7.05E− | 5.81E+ | 9.44E− | 1.62E− | 7.67E+ | 6.21E− | 8.10E− |
| | 03 | 03 | 07 | 03 | 03 | 07 | 03 | 03 | 06 | 03 | 03 | 07 |
| A4-037 | 1.29E+ | 1.50E− | 1.16E− | 5.99E+ | 6.30E− | 1.05E− | 9.83E+ | 4.00E− | 4.07E− | 1.36E+ | 1.20E− | 8.80E− |
| | 05 | 03 | 08 | 04 | 04 | 08 | 04 | 04 | 09 | 05 | 04 | 10 |
| A4-038 | 7.19E+ | 5.00E− | 6.95E− | 1.99E+ | 1.40E− | 7.04E− | 1.51E+ | 1.10E− | 7.29E− | 1.78E+ | 2.80E− | 1.57E− |
| | 03 | 04 | 08 | 05 | 04 | 10 | 05 | 03 | 09 | 05 | 04 | 09 |
| A4-039 | 6.29E+ | 1.77E− | 2.81E− | 3.80E+ | 1.50E− | 3.95E− | 2.78E+ | 1.00E− | 3.60E− | 7.41E+ | 1.82E− | 2.46E− |
| | 03 | 03 | 07 | 03 | 04 | 08 | 03 | 03 | 07 | 03 | 03 | 07 |
| A4-040 | 1.64E+ | 2.80E− | 1.71E− | 6.53E+ | 3.40E− | 5.21E− | 1.01E+ | 2.20E− | 2.19E− | 7.23E+ | 2.20E− | 3.04E− |
| | 05 | 04 | 09 | 03 | 04 | 08 | 05 | 04 | 09 | 04 | 04 | 09 |
| A4-041 | 1.24E+ | 2.45E− | 1.98E− | 1.62E+ | 4.33E− | 2.67E− | 1.74E+ | 1.42E− | 8.15E− | 1.34E+ | 6.16E− | 4.59E− |
| | 03 | 03 | 06 | 05 | 03 | 08 | 05 | 03 | 09 | 05 | 03 | 08 |
| A4-042 | 3.36E+ | 4.20E− | 1.25E− | 6.46E+ | 1.34E− | 2.07E− | 4.68E+ | 1.44E− | 3.08E− | 7.76E+ | 1.18E− | 1.52E− |
| | 03 | 04 | 07 | 03 | 03 | 07 | 03 | 03 | 07 | 03 | 03 | 07 |
| A4-043 | 1.91E+ | 1.04E− | 5.45E− | 1.72E+ | 8.20E− | 4.76E− | 1.44E+ | 4.00E− | 2.78E− | 3.62E+ | 2.80E− | 7.73E− |
| | 05 | 03 | 09 | 05 | 04 | 09 | 05 | 05 | 10 | 04 | 04 | 09 |
| A4-044 | 1.58E+ | 2.40E− | 1.52E− | 5.29E+ | 6.90E− | 1.31E− | 1.43E+ | 1.88E− | 1.31E− | 1.83E+ | 1.37E− | 7.47E− |
| | 05 | 04 | 09 | 04 | 04 | 08 | 05 | 03 | 08 | 05 | 03 | 09 |
| A4-045 | 9.39E+ | 1.00E− | 1.07E− | 8.61E+ | 1.10E− | 1.28E− | 7.19E+ | 2.70E− | 3.76E− | 8.95E+ | 3.70E− | 4.13E− |
| | 03 | 05 | 09 | 03 | 04 | 08 | 03 | 04 | 08 | 03 | 04 | 08 |
| A4-046 | 1.21E+ | 4.55E− | 3.76E− | 1.48E+ | 7.25E− | 4.88E− | 1.67E+ | 2.69E− | 1.61E− | 1.69E+ | 9.33E− | 5.53E− |
| | 05 | 03 | 08 | 05 | 03 | 08 | 05 | 03 | 08 | 05 | 03 | 08 |
| A4-047 | 1.93E+ | 2.00E− | 1.04E− | 1.90E+ | 1.83E− | 9.61E− | 5.05E+ | 1.47E− | 2.91E− | 1.85E+ | 1.44E− | 7.79E− |
| | 05 | 05 | 10 | 04 | 03 | 08 | 04 | 03 | 08 | 05 | 03 | 09 |
| A4-048 | 2.13E+ | 3.10E− | 1.46E− | 8.85E+ | 1.37E− | 1.55E− | 3.26E+ | 1.30E− | 3.99E− | 9.38E+ | 5.10E− | 5.44E− |
| | 03 | 04 | 07 | 03 | 03 | 07 | 03 | 03 | 07 | 03 | 04 | 08 |
| A4-049 | 2.69E+ | 5.60E− | 2.08E− | 1.82E+ | 1.49E− | 8.21E− | 2.08E+ | 1.94E− | 9.31E− | 1.64E+ | 1.58E− | 9.61E− |
| | 04 | 04 | 08 | 05 | 03 | 09 | 04 | 03 | 08 | 05 | 03 | 09 |
| A4-050 | 1.76E+ | 4.30E− | 2.44E− | 2.10E+ | 3.90E− | 1.86E− | 1.94E+ | 4.90E− | 2.52E− | 1.00E+ | 7.00E− | 7.00E− |
| | 05 | 04 | 09 | 04 | 04 | 08 | 05 | 04 | 09 | 04 | 05 | 09 |
| A4-051 | 5.89E+ | 4.66E− | 7.91E− | 3.58E+ | 3.25E− | 9.07E− | 6.70E+ | 4.77E− | 7.12E− | 6.78E+ | 5.07E− | 7.48E− |
| | 03 | 03 | 07 | 03 | 03 | 07 | 03 | 03 | 07 | 03 | 03 | 07 |
| A4-052 | 3.18E+ | 1.51E− | 4.76E− | 1.44E+ | 1.06E− | 7.38E− | 1.04E+ | 7.70E− | 7.38E− | 1.44E+ | 5.60E− | 3.88E− |
| | 04 | 03 | 08 | 05 | 03 | 09 | 05 | 04 | 09 | 03 | 04 | 07 |
| A4-053 | 9.32E+ | 1.46E− | 1.57E− | 1.45E+ | 4.90E− | 3.38E− | 1.85E+ | 4.80E− | 2.60E− | 1.61E+ | 1.21E− | 7.52E− |
| | 04 | 03 | 08 | 05 | 04 | 09 | 05 | 04 | 09 | 05 | 03 | 09 |
| A4-054 | 4.89E+ | 1.80E− | 3.68E− | 1.08E+ | 1.14E− | 1.06E− | 8.66E+ | 2.50E− | 2.89E− | 7.66E+ | 1.63E− | 2.13E− |
| | 03 | 03 | 07 | 03 | 03 | 06 | 03 | 04 | 08 | 03 | 03 | 07 |
| A4-055 | 1.98E+ | 4.40E− | 2.23E− | 1.09E+ | 4.80E− | 4.41E− | 7.88E+ | 3.20E− | 4.06E− | 1.98E+ | 2.20E− | 1.11E− |
| | 05 | 04 | 09 | 05 | 04 | 09 | 04 | 04 | 09 | 05 | 04 | 09 |
| A4-056 | 2.94E+ | 7.79E− | 2.65E− | 2.16E+ | 4.85E− | 2.24E− | 9.15E+ | 8.87E− | 9.69E− | 1.04E+ | 6.48E− | 6.22E− |
| | 04 | 03 | 07 | 04 | 03 | 07 | 04 | 03 | 08 | 04 | 03 | 07 |
| A4-057 | 3.02E+ | 3.10E− | 1.03E− | 8.56E+ | 9.50E− | 1.11E− | 9.19E+ | 4.90E− | 5.33E− | 4.20E+ | 2.70E− | 6.44E− |
| | 03 | 04 | 07 | 03 | 04 | 07 | 03 | 04 | 08 | 03 | 04 | 08 |
| A4-058 | 5.18E+ | 1.73E− | 3.34E− | 9.63E+ | 1.64E− | 1.70E− | 1.96E+ | 1.20E− | 6.11E− | 5.26E+ | 1.29E− | 2.45E− |
| | 04 | 03 | 08 | 04 | 03 | 08 | 05 | 03 | 09 | 04 | 03 | 08 |
| A4-059 | 3.25E+ | 9.40E− | 2.90E− | 1.33E+ | 1.19E− | 8.94E− | 1.17E+ | 6.00E− | 5.14E− | 5.49E+ | 9.10E− | 1.66E− |
| | 04 | 04 | 08 | 05 | 03 | 09 | 05 | 05 | 10 | 04 | 04 | 08 |
| A4-060 | 8.29E+ | 5.50E− | 6.63E− | 1.96E+ | 1.40E− | 7.14E− | 2.54E+ | 1.76E− | 6.92E− | 1.84E+ | 4.50E− | 2.45E− |
| | 04 | 04 | 09 | 05 | 03 | 09 | 04 | 03 | 08 | 05 | 04 | 09 |
| A4-061 | 1.08E+ | 5.70E− | 5.30E− | 7.71E+ | 8.50E− | 1.10E− | 4.77E+ | 1.30E− | 2.73E− | 6.33E+ | 5.50E− | 8.68E− |
| | 03 | 04 | 07 | 03 | 04 | 07 | 03 | 03 | 07 | 03 | 04 | 08 |
| A4-062 | 1.37E+ | 1.90E− | 1.39E− | 1.52E+ | 1.10E− | 7.24E− | 3.28E+ | 6.00E− | 1.83E− | 1.57E+ | 1.00E− | 6.35E− |
| | 05 | 04 | 09 | 05 | 04 | 10 | 04 | 05 | 09 | 05 | 05 | 11 |
| A4-063 | 1.48E+ | 6.50E− | 4.39E− | 1.89E+ | 7.58E− | 4.00E− | 4.91E+ | 4.01E− | 8.16E− | 6.50E+ | 8.34E− | 1.28E− |
| | 04 | 03 | 07 | 05 | 03 | 08 | 04 | 03 | 08 | 04 | 03 | 07 |
| A4-064 | 7.50E+ | 1.70E− | 2.27E− | 6.60E+ | 5.00E− | 7.57E− | 3.05E+ | 1.28E− | 4.20E− | 5.62E+ | 4.60E− | 8.18E− |
| | 03 | 03 | 07 | 03 | 04 | 08 | 03 | 03 | 07 | 03 | 04 | 08 |
| A4-065 | 5.18E+ | 9.50E− | 1.84E− | 1.96E+ | 1.00E− | 5.10E− | 1.65E+ | 1.39E− | 8.44E− | 6.19E+ | 1.55E− | 2.50E− |
| | 03 | 04 | 07 | 04 | 04 | 09 | 05 | 03 | 09 | 04 | 03 | 08 |
| A4-066 | 9.24E+ | 2.00E− | 2.16E− | 1.09E+ | 9.00E− | 8.22E− | 3.07E+ | 1.01E− | 3.28E− | 9.21E+ | 1.91E− | 2.07E− |
| | 04 | 03 | 08 | 05 | 05 | 10 | 04 | 03 | 08 | 04 | 03 | 08 |
| A4-067 | 3.44E+ | 4.40E− | 1.28E− | 1.87E+ | 3.00E− | 1.60E− | 3.32E+ | 3.50E− | 1.06E− | 2.80E+ | 2.00E− | 7.14E− |
| | 03 | 04 | 07 | 03 | 05 | 08 | 03 | 04 | 07 | 03 | 05 | 09 |
| A4-068 | 2.40E+ | 4.36E− | 1.82E− | 1.41E+ | 1.67E− | 1.19E− | 1.09E+ | 3.49E− | 3.19E− | 1.34E+ | 6.08E− | 4.53E− |
| | 04 | 03 | 07 | 05 | 03 | 08 | 05 | 03 | 08 | 05 | 03 | 08 |
| A4-069 | 1.22E+ | 1.42E− | 1.16E− | 5.13E+ | 1.60E− | 3.12E− | 1.05E+ | 9.80E− | 9.34E− | 5.58E+ | 2.00E− | 3.58E− |
| | 05 | 03 | 08 | 03 | 03 | 07 | 05 | 04 | 09 | 04 | 04 | 09 |
| A4-070 | 9.09E+ | 1.90E− | 2.09E− | 8.98E+ | 1.72E− | 1.92E− | 3.38E+ | 1.20E− | 3.55E− | 7.37E+ | 6.40E− | 8.68E− |
| | 03 | 04 | 08 | 03 | 03 | 07 | 03 | 04 | 08 | 03 | 04 | 08 |
| A4-071 | 8.33E+ | 1.37E− | 1.64E− | 2.15E+ | 2.00E− | 9.30E− | 5.70E+ | 1.76E− | 3.09E− | 1.81E+ | 1.71E− | 9.46E− |
| | 04 | 03 | 08 | 04 | 03 | 08 | 04 | 03 | 08 | 05 | 03 | 09 |
| A4-072 | 1.63E+ | 4.40E− | 2.71E− | 5.35E+ | 2.10E− | 3.93E− | 1.40E+ | 1.80E− | 1.29E− | 1.00E+ | 3.80E− | 3.79E− |
| | 04 | 04 | 08 | 04 | 04 | 09 | 05 | 04 | 09 | 05 | 04 | 09 |
| A4-073 | 5.42E+ | 6.19E− | 1.14E− | 5.33E+ | 1.86E− | 3.49E− | 1.56E+ | 2.92E− | 1.88E− | 7.53E+ | 8.62E− | 1.14E− |
| | 03 | 03 | 06 | 03 | 03 | 07 | 03 | 03 | 06 | 03 | 03 | 06 |
| A4-074 | 9.41E+ | 1.81E− | 1.92E− | 1.10E+ | 1.57E− | 1.43E− | 2.82E+ | 1.70E− | 6.03E− | 1.75E+ | 8.10E− | 4.64E− |
| | 04 | 03 | 08 | 05 | 03 | 08 | 04 | 04 | 09 | 05 | 04 | 09 |
| A4-075 | 7.00E+ | 6.70E− | 9.57E− | 9.77E+ | 1.18E− | 1.21E− | 1.02E+ | 9.90E− | 9.74E− | 2.98E+ | 8.60E− | 2.89E− |
| | 04 | 04 | 09 | 04 | 03 | 08 | 05 | 04 | 09 | 04 | 04 | 08 |
| A4-076 | 9.38E+ | 9.20E− | 9.81E− | 7.61E+ | 5.60E− | 7.36E− | 7.36E+ | 6.00E− | 8.16E− | 4.06E+ | 1.50E− | 3.70E− |
| | 03 | 04 | 08 | 03 | 04 | 08 | 03 | 05 | 09 | 03 | 04 | 08 |
| A4-077 | 1.30E+ | 3.80E− | 2.92E− | 9.93E+ | 4.60E− | 4.63E− | 1.98E+ | 3.70E− | 1.87E− | 1.93E+ | 1.90E− | 9.87E− |
| | 05 | 04 | 09 | 04 | 04 | 09 | 05 | 04 | 09 | 05 | 04 | 10 |
| A4-078 | 9.75E+ | 6.00E− | 6.16E− | 1.18E+ | 9.59E− | 8.16E− | 2.88E+ | 6.18E− | 2.15E− | 1.77E+ | 9.32E− | 5.27E− |
| | 04 | 03 | 08 | 05 | 03 | 08 | 04 | 03 | 07 | 05 | 03 | 08 |
| A4-079 | 1.18E+ | 1.90E− | 1.62E− | 4.35E+ | 1.76E− | 4.04E− | 1.34E+ | 5.10E− | 3.82E− | 3.93E+ | 8.30E− | 2.11E− |
| | 03 | 03 | 06 | 03 | 03 | 07 | 03 | 04 | 07 | 03 | 04 | 07 |
| A4-080 | 7.22E+ | 1.38E− | 1.91E− | 9.45E+ | 1.47E− | 1.56E− | 1.46E+ | 7.40E− | 5.08E− | 9.52E+ | 1.94E− | 2.04E− |
| | 03 | 03 | 07 | 04 | 03 | 08 | 05 | 04 | 09 | 04 | 03 | 08 |
| A4-081 | 1.74E+ | 1.05E− | 6.05E− | 3.48E+ | 1.70E− | 4.89E− | 9.59E+ | 1.31E− | 1.37E− | 1.17E+ | 1.46E− | 1.25E− |
| | 05 | 03 | 09 | 04 | 04 | 09 | 04 | 03 | 08 | 05 | 03 | 08 |
| A4-082 | 3.64E+ | 1.70E− | 4.67E− | 5.03E+ | 1.60E− | 3.18E− | 3.29E+ | 4.00E− | 1.22E− | 5.38E+ | 5.00E− | 9.29E− |
| | 03 | 04 | 08 | 03 | 04 | 08 | 03 | 04 | 07 | 03 | 04 | 08 |
| A4-083 | 1.74E+ | 7.37E− | 4.24E− | 3.64E+ | 3.44E− | 9.44E− | 7.12E+ | 6.57E− | 9.23E− | 9.25E+ | 2.87E− | 3.10E− |
| | 05 | 03 | 08 | 03 | 03 | 07 | 04 | 03 | 08 | 04 | 03 | 08 |
| A4-084 | 1.38E+ | 1.77E− | 1.28E− | 7.32E+ | 1.63E− | 2.23E− | 1.32E+ | 3.00E− | 2.28E− | 1.65E+ | 6.00E− | 3.64E− |
| | 05 | 03 | 08 | 04 | 03 | 08 | 05 | 04 | 09 | 05 | 05 | 10 |
| A4-085 | 3.79E+ | 4.10E− | 1.08E− | 3.77E+ | 1.86E− | 4.94E− | 2.24E+ | 5.00E− | 2.23E− | 9.94E+ | 1.91E− | 1.92E− |
| | 03 | 04 | 07 | 03 | 03 | 07 | 03 | 04 | 07 | 03 | 03 | 07 |
| A4-086 | 7.89E+ | 6.00E− | 7.60E− | 1.12E+ | 1.28E− | 1.15E− | 1.79E+ | 2.60E− | 1.45E− | 4.64E+ | 1.07E− | 2.31E− |
| | 04 | 05 | 10 | 05 | 03 | 08 | 05 | 04 | 09 | 04 | 03 | 08 |
| A4-087 | 1.15E+ | 1.20E− | 1.05E− | 1.30E+ | 4.30E− | 3.31E− | 5.57E+ | 2.30E− | 4.13E− | 1.77E+ | 3.00E− | 1.69E− |
| | 05 | 04 | 09 | 05 | 04 | 09 | 04 | 04 | 09 | 05 | 04 | 09 |
| A4-088 | 8.44E+ | 4.54E− | 5.38E− | 4.10E+ | 9.93E− | 2.42E− | 7.57E+ | 1.77E− | 2.34E− | 7.26E+ | 1.46E− | 2.01E− |
| | 03 | 03 | 07 | 03 | 03 | 06 | 03 | 03 | 07 | 03 | 03 | 07 |
| A4-089 | 1.21E+ | 5.40E− | 4.48E− | 1.87E+ | 8.00E− | 4.28E− | 1.79E+ | 4.10E− | 2.29E− | 6.08E+ | 9.90E− | 1.63E− |
| | 05 | 04 | 09 | 05 | 04 | 09 | 05 | 04 | 09 | 03 | 04 | 07 |
| A4-090 | 7.53E+ | 1.09E− | 1.45E− | 1.99E+ | 1.36E− | 6.83E− | 6.04E+ | 1.50E− | 2.49E− | 1.53E+ | 1.86E− | 1.21E− |
| | 04 | 03 | 08 | 05 | 03 | 09 | 04 | 03 | 08 | 04 | 03 | 07 |
| A4-091 | 9.16E+ | 9.10E− | 9.94E− | 8.01E+ | 1.22E− | 1.52E− | 5.79E+ | 2.00E− | 3.45E− | 6.69E+ | 3.00E− | 4.49E− |
| | 03 | 04 | 08 | 03 | 03 | 07 | 03 | 04 | 08 | 03 | 05 | 09 |
| A4-092 | 1.88E+ | 2.00E− | 1.06E− | 5.51E+ | 6.00E− | 1.09E− | 8.49E+ | 9.00E− | 1.06E− | 5.16E+ | 1.00E− | 1.94E− |
| | 05 | 05 | 10 | 04 | 05 | 09 | 04 | 05 | 09 | 04 | 05 | 10 |
| A4-093 | 1.66E+ | 1.96E− | 1.18E− | 2.24E+ | 1.03E− | 4.60E− | 7.08E+ | 1.10E− | 1.55E− | 1.39E+ | 1.14E− | 8.21E− |
| | 05 | 03 | 08 | 04 | 03 | 08 | 04 | 03 | 08 | 05 | 03 | 09 |
| A4-094 | 6.71E+ | 5.80E− | 8.65E− | 7.13E+ | 1.39E− | 1.95E− | 9.60E+ | 4.30E− | 4.48E− | 9.68E+ | 1.66E− | 1.71E− |
| | 03 | 04 | 08 | 03 | 03 | 07 | 03 | 04 | 08 | 03 | 03 | 07 |
| A4-095 | 1.16E+ | 2.60E− | 2.25E− | 1.35E+ | 5.70E− | 4.23E− | 1.80E+ | 1.14E− | 6.34E− | 9.18E+ | 1.33E− | 1.45E− |
| | 05 | 04 | 09 | 05 | 04 | 09 | 05 | 03 | 09 | 04 | 03 | 08 |
| A4-096 | 1.10E+ | 1.69E− | 1.54E− | 1.10E+ | 1.07E− | 9.77E− | 1.25E+ | 6.40E− | 5.14E− | 2.51E+ | 1.23E− | 4.89E− |
| | 05 | 03 | 08 | 05 | 03 | 09 | 05 | 04 | 09 | 04 | 03 | 08 |
| A4-097 | 1.55E+ | 1.92E− | 1.24E− | 5.37E+ | 7.80E− | 1.45E− | 1.31E+ | 8.16E− | 6.23E− | 9.41E+ | 5.44E− | 5.78E− |
| | 05 | 03 | 08 | 04 | 04 | 08 | 05 | 03 | 08 | 03 | 03 | 07 |
| A4-098 | 7.74E+ | 1.80E− | 2.33E− | 6.66E+ | 1.60E− | 2.40E− | 7.30E+ | 6.00E− | 8.22E− | 9.03E+ | 1.17E− | 1.30E− |
| | 03 | 04 | 08 | 03 | 04 | 08 | 03 | 04 | 08 | 03 | 03 | 07 |
| A4-099 | 1.94E+ | 5.00E− | 2.57E− | 1.97E+ | 2.10E− | 1.06E− | 1.48E+ | 3.00E− | 2.03E− | 9.80E+ | 4.10E− | 4.19E− |
| | 05 | 04 | 09 | 05 | 04 | 09 | 05 | 05 | 10 | 04 | 04 | 09 |
| A4-100 | 9.83E+ | 3.56E− | 3.62E− | 8.75E+ | 6.49E− | 7.42E− | 1.65E+ | 1.26E− | 7.64E− | 1.84E+ | 1.24E− | 6.75E− |
| | 04 | 03 | 08 | 04 | 03 | 08 | 05 | 03 | 09 | 05 | 03 | 09 |
| A4-101 | 7.97E+ | 1.29E− | 1.62E− | 9.07E+ | 1.31E− | 1.44E− | 6.70E+ | 9.00E− | 1.34E− | 4.80E+ | 3.40E− | 7.08E− |
| | 03 | 03 | 07 | 03 | 03 | 07 | 03 | 05 | 08 | 03 | 04 | 08 |
| A4-102 | 7.63E+ | 6.00E− | 7.86E− | 1.66E+ | 1.91E− | 1.15E− | 1.51E+ | 4.65E− | 3.08E− | 1.73E+ | 7.00E− | 4.04E− |
| | 04 | 04 | 09 | 05 | 03 | 08 | 05 | 03 | 08 | 05 | 03 | 08 |
| A4-103 | 5.19E+ | 1.82E− | 3.51E− | 1.78E+ | 1.42E− | 7.97E− | 1.32E+ | 1.07E− | 8.12E− | 5.10E+ | 1.37E− | 2.69E− |
| | 04 | 03 | 08 | 05 | 03 | 09 | 04 | 03 | 08 | 04 | 03 | 08 |
| A4-104 | 2.86E+ | 3.00E− | 1.05E− | 4.13E+ | 4.30E− | 1.04E− | 2.06E+ | 9.30E− | 4.51E− | 8.42E+ | 3.70E− | 4.40E− |
| | 03 | 05 | 08 | 03 | 04 | 07 | 03 | 04 | 07 | 03 | 04 | 08 |
| A4-105 | 1.80E+ | 3.91E− | 2.17E− | 1.59E+ | 5.43E− | 3.42E− | 1.11E+ | 1.89E− | 1.70E− | 9.96E+ | 1.39E− | 1.40E− |
| | 05 | 03 | 08 | 05 | 03 | 08 | 05 | 03 | 08 | 04 | 03 | 08 |
| A4-106 | 1.20E+ | 1.91E− | 1.60E− | 1.42E+ | 3.70E− | 2.61E− | 6.66E+ | 1.10E− | 1.65E− | 1.98E+ | 4.20E− | 2.12E− |
| | 05 | 03 | 08 | 05 | 04 | 09 | 04 | 04 | 09 | 05 | 04 | 09 |
| A4-107 | 1.19E+ | 1.01E− | 8.48E− | 8.41E+ | 1.26E− | 1.50E− | 2.12E+ | 3.56E− | 1.68E− | 5.02E+ | 3.53E− | 7.03E− |
| | 03 | 03 | 07 | 03 | 03 | 07 | 03 | 03 | 06 | 03 | 03 | 07 |
| A4-108 | 1.07E+ | 8.50E− | 7.94E− | 1.91E+ | 2.40E− | 1.26E− | 1.72E+ | 6.10E− | 3.54E− | 1.48E+ | 1.05E− | 7.12E− |
| | 05 | 04 | 09 | 05 | 04 | 09 | 05 | 04 | 09 | 05 | 03 | 09 |
| A4-109 | 1.54E+ | 3.50E− | 2.27E− | 1.17E+ | 2.70E− | 2.31E− | 1.68E+ | 7.10E− | 4.24E− | 5.88E+ | 2.20E− | 3.74E− |
| | 05 | 04 | 09 | 05 | 04 | 09 | 04 | 04 | 08 | 04 | 04 | 09 |
| A4-110 | 6.93E+ | 5.96E− | 8.60E− | 4.69E+ | 4.17E− | 8.90E− | 4.04E+ | 1.40E− | 3.47E− | 9.27E+ | 3.50E− | 3.78E− |
| | 03 | 03 | 07 | 03 | 03 | 07 | 03 | 03 | 07 | 03 | 04 | 08 |
| A4-111 | 7.45E+ | 1.40E− | 1.88E− | 5.69E+ | 2.00E− | 3.51E− | 1.25E+ | 2.50E− | 2.00E− | 8.53E+ | 9.00E− | 1.06E− |
| | 04 | 04 | 09 | 04 | 04 | 09 | 05 | 04 | 09 | 04 | 05 | 09 |
| A4-112 | 1.88E+ | 2.10E− | 1.11E− | 4.08E+ | 1.39E− | 3.41E− | 1.42E+ | 5.20E− | 3.66E− | 1.30E+ | 4.12E− | 3.16E− |
| | 05 | 04 | 09 | 04 | 03 | 08 | 05 | 03 | 08 | 05 | 03 | 08 |
| A4-113 | 6.15E+ | 2.00E− | 3.25E− | 1.17E+ | 1.09E− | 9.34E− | 9.72E+ | 5.10E− | 5.25E− | 6.66E+ | 1.45E− | 2.18E− |
| | 03 | 05 | 09 | 03 | 03 | 07 | 03 | 04 | 08 | 03 | 03 | 07 |
| A4-114 | 9.40E+ | 1.70E− | 1.81E− | 1.13E+ | 2.20E− | 1.95E− | 2.61E+ | 1.23E− | 4.72E− | 1.55E+ | 1.10E− | 7.11E− |
| | 04 | 04 | 09 | 05 | 04 | 09 | 03 | 03 | 07 | 05 | 03 | 09 |

### B. Sequence Optimization of Anti-AAV Nanobodies

### Example 6: Humanization Engineering and Affinity Characterization of Anti-AAV Nanobodies

### Humanization Strategy 1: Framework Residue Substitution

The amino acid sequences of parental nanobodies A4-015 and A4-036 were aligned against human germline immunoglobulin sequences. Non-human residues within framework regions (FRs) were systematically identified, and selected substitutions were introduced via site-directed mutagenesis to match human consensus residues. This process yielded four humanized variants: A4-H3, A4-H4, A4-H5, and A4-H6. The engineered constructs were subsequently expressed in a suitable host system and purified to homogeneity using standard chromatographic methods.

Humanization Strategy 2: CDR Grafting onto Human Germline Frameworks

Complementarity-determining regions (CDRs) from the parental A4-003 nanobody were grafted onto human germline framework regions identified through sequence homology analysis. Synthetic genes encoding the chimeric sequences (designated A4-H1 and A4-H2) were chemically synthesized, cloned into expression vectors, and recombinantly expressed. Purification of the humanized nanobodies was performed using established biochemical protocols.
(1) Humanized nanobodies were expressed in TB medium. A 5% (v/v) inoculum of transformed host cells was cultured at 37° C. for 3-5 hours. Protein expression was induced by adding isopropyl β-D-thiogalactoside (IPTG) to a final concentration of 0.25 mM, followed by overnight incubation at 18-20° C.
(2) Post-induction, cells were pelleted by centrifugation at 4000 rpm for 20 minutes at 4° C. to obtain wet biomass containing the humanized nanobodies.
(3) The harvested wet cells were resuspended in lysis buffer (10 mM imidazole, 500 mM NaCl, 20 mM phosphate buffer, pH 7.4) at a ratio of 1:10 (w/v). Cell disruption was performed using a high-pressure homogenizer at 700 bar for 3-5 passes.
(4) The lysate was centrifuged at 10,000 rpm for 20 minutes at 4° C., and the supernatant containing soluble nanobodies was collected.
(5) The clarified supernatant was filtered through a 0.45 μm membrane and applied to a pre-equilibrated Ni Sepharose High Performance affinity column (GE Healthcare, USA). The column was washed with 5-10 column volumes of equilibration buffer (identical to lysis buffer) followed by wash buffer (20 mM imidazole, 500 mM NaCl, 20 mM phosphate buffer, pH 7.4). Bound nanobodies were eluted with elution buffer (250 mM imidazole, 500 mM NaCl, 20 mM phosphate buffer, pH 7.4).
(6) Purified nanobodies were analyzed by SDS-PAGE electrophoresis. Fractions with ≥95% purity were pooled, and protein concentration was determined using a BCA protein assay kit.

### Surface Plasmon Resonance (SPR) Analysis of Binding Kinetics Between Humanized Nanobodies and AAV Serotypes.

Recombinant adeno-associated virus (AAV) particles were covalently immobilized onto a CM5 sensor chip via amine coupling to a surface density of 500-800RU. Six serial dilutions of nanobody analytes (concentration range: 1-100 nM) were injected over the immobilized AAV surfaces at a constant flow rate of 45 μL/min using HBS-EP+ buffer (10 mM HEPES, 150 mM NaCl, 3 mM EDTA, 0.05% (v/v) Surfactant P20, pH 7.4). The sensor chip was regenerated using 10 mM glycine-HCl (pH 1.5) between binding cycles. Kinetic parameters (Kₐ, Kₐ, K_{D}) were determined by fitting the sensorgrams to a 1:1 binding model, as shown in Table 3.

Humanization of the parental nanobodies (e.g., A4-003) via CDR grafting (A4-H1/H2) or framework substitution (A4-H3/H4) did not significantly alter binding affinity to AAV serotypes, as evidenced by K_{D} values remaining within the same order of magnitude. For instance, the parental A4-003 exhibited K_{D} values of 1.2×10⁻⁹ M, 6.07×10⁻⁹ M, 1.84×10⁻⁷ M, and 8.24×10⁻⁹ M against AAV8, AAV2, AAV9, and AAV5, respectively. Corresponding humanized variants A4-H1 and A4-H2 maintained comparable affinity profiles.

TABLE 3
Kinetic Parameters of Humanized Nanobodies

| | Antigen | | | | | | | | | | | |
|---|---|---|---|---|---|---|---|---|---|---|---|---|
| Nanobody | AAV8 | | | AAV2 | | | AAV9 | | | AAV5 | | |
| Name | kₐ(1/MS) | k_{d}(1/S) | K_{D}(M) | kₐ(1/MS) | k_{d}(1/S) | K_{D}(M) | kₐ(1/MS) | k_{d}(1/S) | K_{D}(M) | kₐ(1/MS) | k_{d}(1/S) | K_{D}(M) |
| A4-H1 | 1.98E+ | 1.35E− | 6.82E− | 1.63E+ | 1.39E− | 8.51E− | 5.42E+ | 6.90E− | 1.27E− | 1.75E+ | 1.53E− | 8.76E− |
| | 05 | 03 | 09 | 05 | 03 | 09 | 03 | 04 | 07 | 05 | 03 | 09 |
| A4-H2 | 1.41E+ | 2.80E− | 1.98E− | 6.89E+ | 4.10E− | 5.95E− | 4.70E+ | 1.88E− | 4.00E− | 1.63E+ | 1.39E− | 8.51E− |
| | 05 | 04 | 09 | 04 | 04 | 09 | 03 | 03 | 07 | 05 | 03 | 09 |
| A4-H3 | 1.34E+ | 1.28E− | 9.59E− | 6.94E+ | 1.34E− | 1.93E− | 1.69E+ | 7.30E− | 4.32E− | 1.57E+ | 4.80E− | 3.05E− |
| | 05 | 03 | 09 | 04 | 03 | 08 | 05 | 04 | 09 | 05 | 04 | 09 |
| A4-H4 | 1.32E+ | 1.25E− | 9.44E− | 9.90E+ | 1.70E− | 1.72E− | 1.32E+ | 7.80E− | 5.91E− | 1.12E+ | 3.20E− | 2.86E− |
| | 05 | 03 | 09 | 04 | 03 | 08 | 05 | 04 | 09 | 05 | 04 | 09 |
| A4-H5 | 4.30E+ | 1.30E− | 3.02E− | 1.85E+ | 1.52E− | 8.23E− | 3.05E+ | 5.80E− | 1.90E− | 1.85E+ | 1.52E− | 8.23E− |
| | 03 | 03 | 07 | 03 | 03 | 07 | 03 | 03 | 06 | 03 | 03 | 07 |
| A4-H6 | 4.76E+ | 1.64E− | 3.44E− | 7.57E+ | 5.65E− | 7.46E− | 1.53E+ | 1.77E− | 1.16E− | 7.57E+ | 5.65E− | 7.46E− |
| | 03 | 03 | 07 | 03 | 03 | 07 | 03 | 03 | 06 | 03 | 03 | 07 |

### Example 7: Multivalent Nanobody Engineering and Affinity Characterization

### 1. Bivalent Nanobody Preparation

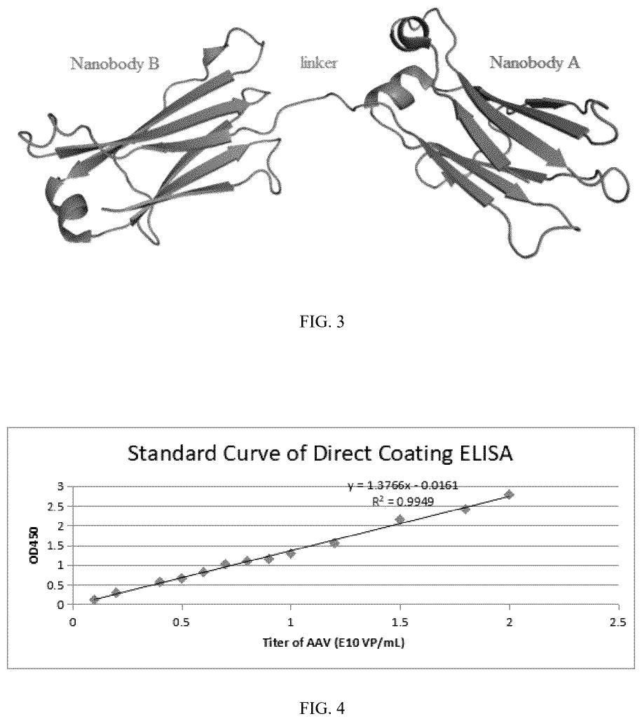

Amino acid sequences of bivalent polypeptides specific for AAV were engineered, including A4-B1, A4-B2, A4-B3, and A4-B4 (depicted in FIG. 3). Each construct consists of a C-terminal nanobody A, a 15-residue Gly/Ser-rich linker, and an N-terminal nanobody B, with nanobodies A and B being either identical or distinct. Representative examples include:
A4-B1: nanobody A=A4-015; nanobody B is identical to A.
A4-B2: nanobody A=A4-014; nanobody B=A4-012.
A4-B3: nanobody A=A4-H1; nanobody B is identical to A.
A4-B4: nanobody A=A4-H1; nanobody B=A4-H4.

The DNA sequences encoding the bivalent polypeptides were chemically synthesized and ligated into the pET23a expression vector to generate recombinant plasmids. The plasmids were transformed into E. coli competent cells to establish recombinant strains for bivalent polypeptide expression. Expression and purification followed the protocols described in Example 6.

### 2. Trivalent Nanobody Preparation

Amino acid sequences of trivalent polypeptides specific for AAV were engineered, including A4-C1 to A4-C9. Each construct is structured as nanobody A-(Gly₄Ser)₃-nanobody B-(Gly₄Ser)₃-nanobody C, where nanobodies A, B, and C may be identical or distinct, and their order is adjustable. Representative designs include:
A4-C1: A=B=C=A4-057;
A4-C2: A=B=C=A4-055;
A4-C3: A=B=A4-015, C=A4-054;
A4-C4: A=B=A4-014, C=A4-054;
A4-C5: A=C=A4-056, B=A4-051;
A4-C6: A=A4-053, B=A4-052, C=A4-051;
A4-C7: A=A4-051, B=A4-053, C=A4-049;
A4-C8: A=A4-053, B=A4-051, C=A4-052;
A4-C9: A=A4-051, B=A4-052, C=A4-053.

The encoding DNA sequences were chemically synthesized, cloned into the pET23a expression vector, and transformed into E. coli cells to generate recombinant strains. Expression and purification of trivalent polypeptides were performed as described in Example 6.

### 3. Tetravalent Nanobody Preparation

Amino acid sequences of tetravalent polypeptides specific for AAV were engineered, including A4-D1 to A4-D13. Each construct is structured as nanobody A-(Gly₄Ser)₃-nanobody B-(Gly₄Ser)₃-nanobody C-(Gly₄Ser)₃-nanobody D, where nanobodies A, B, C, and D may be identical or distinct, and their order is adjustable. Representative designs include:
A4-D1: A=B=C=D=A4-043;
A4-D2: A=B=C=D=A4-044;
A4-D3: A=B=C=A4-043, D=A4-045;
A4-D4: A=C=D=A4-043, B=A4-045;
A4-D5: A=B=D=A4-046, C=A4-047;
A4-D6: A=B=A4-067, C=A4-068, D=A4-069;
A4-D7: A=D=A4-067, B=A4-068, C=A4-069;
A4-D8: B=D=A4-081, A=A4-082, C=A4-076;
A4-D9: B=C=A4-081, A=A4-076, D=A4-067;
A4-D10: A=A4-043, B=A4-036, C=A4-025, D=A4-033;
A4-D11: A=A4-045, B=A4-033, C=A4-025, D=A4-028;
A4-D12: A=A4-033, B=A4-043, C=A4-036, D=A4-025;
A4-D13: A=A4-025, B=A4-033, C=A4-043, D=A4-036.

The encoding DNA sequences were chemically synthesized, cloned into the pET23a expression vector, and transformed into E. coli cells to generate recombinant strains. Expression and purification of tetravalent polypeptides were performed as described in Example 6.

### SPR Analysis of Binding Affinity for Multivalent Polypeptides to AAV.

Surface plasmon resonance (SPR) was used to characterize the binding affinity of multivalent polypeptides to AAV. AAV was immobilized onto a CM5 sensor chip via amine coupling to a surface density of 500-800 RU. Five serial dilutions of nanobodies (concentration range: 1-50 nM) were injected over the immobilized AAV at a constant flow rate of 45 μL/min using HBS-EP+ buffer. The chip was regenerated with 10 mM glycine-HCl (pH 1.5) between runs. Kinetic parameters were calculated by fitting the binding curves obtained at different nanobody concentrations (Table 4).

Compared with monovalent nanobodies, multivalent forms exhibited significant affinity enhancement toward AAV antigens, characterized by decreased dissociation rate constants (k_{d}) and slower dissociation kinetics. The affinity improvement ranged from several-fold to hundreds-fold, as reflected by the reduced K_{D} values.

TABLE 4
Kinetic Parameters of Multivalent Nanobodies

| | Antigen | | | | | | | | | | | |
|---|---|---|---|---|---|---|---|---|---|---|---|---|
| Nanobody | AAV8 | | | AAV2 | | | AAV9 | | | AAV5 | | |
| Name | kₐ(1/MS) | k_{d}(1/S) | K_{D}(M) | kₐ(1/MS) | k_{d}(1/S) | K_{D}(M) | kₐ(1/MS) | k_{d}(1/S) | K_{D}(M) | kₐ(1/MS) | k_{d}(1/S) | K_{D}(M) |
| A4-B1 | 9.45E+ | 1.54E− | 1.63E− | 8.91E+ | 1.47E− | 1.65E− | 1.96E+ | 1.40E− | 7.15E− | 1.21E+ | 1.23E− | 1.02E− |
| | 04 | 04 | 09 | 04 | 04 | 09 | 05 | 04 | 10 | 05 | 05 | 10 |
| A4-B2 | 1.38E+ | 1.40E− | 1.02E− | 9.26E+ | 5.00E− | 5.40E− | 9.58E+ | 8.00E− | 8.35E− | 1.59E+ | 1.17E− | 7.38E− |
| | 05 | 04 | 09 | 04 | 04 | 09 | 04 | 04 | 09 | 05 | 03 | 09 |
| A4-B3 | 9.58E+ | 8.00E− | 8.35E− | 8.16E+ | 2.00E− | 2.45E− | 4.68E+ | 9.50E− | 2.03E− | 1.80E+ | 1.00E− | 5.56E− |
| | 04 | 05 | 10 | 04 | 05 | 10 | 04 | 04 | 08 | 05 | 04 | 10 |
| A4-B4 | 1.51E+ | 5.00E− | 3.32E− | 9.58E+ | 8.00E− | 8.35E− | 1.20E+ | 6.30E− | 5.26E− | 1.84E+ | 1.60E− | 8.72E− |
| | 05 | 05 | 10 | 04 | 05 | 10 | 05 | 04 | 09 | 05 | 04 | 10 |
| A4-C1 | 1.78E+ | 1.64E− | 9.20E− | 1.74E+ | 8.60E− | 4.94E− | 9.69E+ | 1.00E− | 1.03E− | 1.04E+ | 1.38E− | 1.32E− |
| | 05 | 03 | 09 | 05 | 04 | 09 | 03 | 05 | 09 | 05 | 03 | 08 |
| A4-C2 | 1.39E+ | 5.00E− | 3.59E− | 2.23E+ | 1.40E− | 6.29E− | 2.57E+ | 1.25E− | 4.86E− | 1.94E+ | 1.34E− | 6.91E− |
| | 05 | 05 | 10 | 05 | 04 | 10 | 05 | 04 | 10 | 05 | 04 | 10 |
| A4-C3 | 7.49E+ | 4.00E− | 5.34E− | 1.51E+ | 5.60E− | 3.70E− | 1.83E+ | 9.00E− | 4.91E− | 1.43E+ | 9.00E− | 6.28E− |
| | 04 | 05 | 10 | 05 | 04 | 09 | 05 | 05 | 10 | 05 | 05 | 10 |
| A4-C4 | 1.06E+ | 2.50E− | 2.36E− | 8.72E+ | 1.90E− | 2.18E− | 1.65E+ | 3.10E− | 1.87E− | 1.80E+ | 1.21E− | 6.72E− |
| | 05 | 04 | 09 | 04 | 04 | 09 | 05 | 04 | 09 | 05 | 03 | 09 |
| A4-C5 | 1.83E+ | 9.10E− | 4.97E− | 1.63E+ | 5.70E− | 3.50E− | 1.19E+ | 2.00E− | 1.68E− | 1.65E+ | 1.30E− | 7.86E− |
| | 05 | 04 | 09 | 05 | 04 | 09 | 05 | 04 | 09 | 05 | 03 | 09 |
| A4-C6 | 1.65E+ | 4.40E− | 2.67E− | 9.75E+ | 7.00E− | 7.18E− | 4.54E+ | 2.50E− | 5.50E− | 1.77E+ | 1.39E− | 7.85E− |
| | 05 | 04 | 09 | 04 | 05 | 10 | 04 | 05 | 10 | 05 | 04 | 10 |
| A4-C7 | 1.27E+ | 4.70E− | 3.70E− | 8.46E+ | 4.00E− | 4.73E− | 1.37E+ | 1.75E− | 1.27E− | 4.37E+ | 3.20E− | 7.32E− |
| | 05 | 04 | 09 | 04 | 05 | 10 | 05 | 05 | 10 | 05 | 05 | 11 |
| A4-C8 | 1.32E+ | 3.70E− | 2.81E− | 1.33E+ | 8.00E− | 6.02E− | 1.79E+ | 5.00E− | 2.79E− | 1.88E+ | 1.30E− | 6.90E− |
| | 05 | 04 | 09 | 05 | 05 | 10 | 05 | 05 | 10 | 05 | 04 | 10 |
| A4-C9 | 1.14E+ | 1.30E− | 1.14E− | 1.17E+ | 1.00E− | 8.57E− | 1.94E+ | 1.30E− | 6.69E− | 6.43E+ | 5.00E− | 7.77E− |
| | 05 | 04 | 09 | 05 | 04 | 10 | 05 | 04 | 10 | 04 | 05 | 10 |
| A4-D1 | 3.34E+ | 1.81E− | 5.43E− | 4.96E+ | 3.30E− | 6.65E− | 5.82E+ | 3.10E− | 5.33E− | 3.82E+ | 2.20E− | 5.76E− |
| | 05 | 05 | 11 | 04 | 06 | 11 | 04 | 06 | 11 | 04 | 06 | 11 |
| A4-D2 | 4.39E+ | 3.60E− | 8.21E− | 3.08E+ | 6.60E− | 2.15E− | 2.96E+ | 9.00E− | 3.04E− | 5.72E+ | 5.70E− | 9.96E− |
| | 04 | 06 | 11 | 04 | 06 | 10 | 04 | 06 | 10 | 04 | 06 | 11 |
| A4-D3 | 4.14E+ | 1.50E− | 3.63E− | 4.76E+ | 1.52E− | 3.20E− | 4.53E+ | 3.20E− | 7.07E− | 1.73E+ | 3.60E− | 2.09E− |
| | 04 | 06 | 11 | 04 | 05 | 10 | 04 | 06 | 11 | 04 | 06 | 10 |
| A4-D4 | 2.12E+ | 1.70E− | 8.02E− | 3.44E+ | 1.11E− | 3.23E− | 2.88E+ | 2.10E− | 7.30E− | 2.63E+ | 1.73E− | 6.57E− |
| | 04 | 06 | 11 | 04 | 05 | 10 | 04 | 06 | 11 | 04 | 05 | 10 |
| A4-D5 | 5.98E+ | 5.00E− | 8.36E− | 3.44E+ | 1.38E− | 4.02E− | 3.69E+ | 4.30E− | 1.17E− | 4.21E+ | 1.34E− | 3.18E− |
| | 04 | 06 | 11 | 04 | 05 | 10 | 04 | 06 | 10 | 04 | 05 | 10 |
| A4-D6 | 1.60E+ | 1.63E− | 1.02E− | 1.05E+ | 4.90E− | 4.65E− | 2.19E+ | 3.40E− | 1.55E− | 4.33E+ | 1.18E− | 2.73E− |
| | 04 | 05 | 09 | 04 | 05 | 09 | 04 | 06 | 10 | 04 | 05 | 10 |
| A4-D7 | 1.27E+ | 1.79E− | 1.41E− | 1.29E+ | 1.65E− | 1.28E− | 1.43E+ | 9.40E− | 6.58E− | 2.27E+ | 4.90E− | 2.16E− |
| | 04 | 05 | 09 | 04 | 05 | 09 | 04 | 06 | 10 | 04 | 06 | 10 |
| A4-D8 | 4.82E+ | 1.10E− | 2.28E− | 2.21E+ | 8.40E− | 3.80E− | 4.22E+ | 1.32E− | 3.13E− | 1.32E+ | 1.96E− | 1.49E− |
| | 04 | 05 | 10 | 04 | 06 | 10 | 04 | 05 | 10 | 04 | 05 | 09 |
| A4-D9 | 3.11E+ | 9.90E− | 3.18E− | 3.14E+ | 4.50E− | 1.44E− | 2.60E+ | 1.94E− | 7.45E− | 2.29E+ | 2.30E− | 1.00E− |
| | 04 | 06 | 10 | 04 | 06 | 10 | 04 | 05 | 10 | 04 | 06 | 10 |
| A4-D10 | 3.07E+ | 1.66E− | 5.42E− | 5.94E+ | 1.90E− | 3.20E− | 3.11E+ | 1.90E− | 6.11E− | 1.73E+ | 8.30E− | 4.81E− |
| | 04 | 05 | 10 | 04 | 05 | 10 | 04 | 06 | 11 | 04 | 06 | 10 |
| A4-D11 | 4.70E+ | 1.50E− | 3.19E− | 3.06E+ | 1.71E− | 5.58E− | 2.65E+ | 4.80E− | 1.81E− | 1.22E+ | 1.40E− | 1.15E− |
| | 04 | 05 | 10 | 04 | 05 | 10 | 04 | 06 | 10 | 04 | 05 | 09 |
| A4-D12 | 2.48E+ | 6.50E− | 2.62E− | 3.61E+ | 1.77E− | 4.91E− | 5.40E+ | 3.00E− | 5.55E− | 3.86E+ | 1.70E− | 4.40E− |
| | 04 | 06 | 10 | 04 | 05 | 10 | 04 | 06 | 11 | 04 | 05 | 10 |
| A4-D13 | 1.94E+ | 6.90E− | 3.55E− | 3.55E+ | 6.80E− | 1.91E− | 5.52E+ | 2.60E− | 4.71E− | 1.87E+ | 1.31E− | 7.01E− |
| | 04 | 06 | 10 | 04 | 06 | 10 | 04 | 06 | 11 | 04 | 05 | 10 |

### Example 8

### Amino Acid Modification at the N-Terminus and C-Terminus of Nanobodies and Affinity Determination

Through techniques such as genetic engineering, post-translational modification, and protein engineering, one or more amino acid tags (e.g., His tag, biotin ligase tag, HA tag, SUMO tag, GST tag, thiol tag, Poly-lysine tag, thrombin tag, enterokinase tag, etc.) may be appended individually or concurrently to the N-terminus and/or C-terminus of nanobodies or multivalent nanobodies. If necessary, peptide linkers may be interposed between the amino acid tags and the nanobody to enable efficient expression, modification, purification, and functionalization of the nanobodies, without compromising their antigen-binding capability.

Nanobody variants with C-terminal amino acid modifications were designed, including A4-T1 (SEQ ID No: 147), A4-T2 (SEQ ID No: 148), and A4-L1 (SEQ ID No: 149). Specifically:
A4-T1 comprises A4-017 with a C-terminal 6×His tag;
A4-T2 comprises A4-017 with a C-terminal GGGGS linker followed by a 6×His tag;
A4-L1 comprises A4-017 with a C-terminal IgA hinge (sequence: SerThrProProThrProSerProSerThrProPro) followed by a 6×His tag.

Nanobody variants with N-terminal amino acid modifications were designed, including A4-M1 (SEQ ID No: 150) and A4-M2 (SEQ ID No: 151). Specifically:
A4-M1 comprises A4-017 with an N-terminal amino acid sequence “MetAlaGln”;
A4-M2 comprises A4-017 with an N-terminal methionine (Met).

The DNA sequences encoding the aforementioned nanobody variants were artificially synthesized. The resulting DNA fragments were ligated into the pET23a vector to generate recombinant plasmids, which were subsequently transformed into E. coli to yield engineered strains harboring the modified nanobodies.

Subsequently, expression and purification of the modified nanobodies were performed as described in Example 6.

### SPR Analysis of Binding Affinity for Modified Nanobodies to AAV.

AAV was immobilized onto a CM5 sensor chip via amine coupling at a surface density of 500-800 RU. Five serial dilutions of the nanobodies (concentration range: 1-50 nM) were injected over the immobilized AAV at a constant flow rate of 45 μL/min in HBS-EP+ buffer. Between successive runs, the chip was regenerated using 10 mM glycine-HCl (pH 1.5). Kinetic parameters were calculated by fitting the binding curves obtained at the various nanobody concentrations (see Table 5). As shown in Table 5, relative to the parental sequence A4-017, the modified nanobodies retained substantially unaltered affinity for the antigen AAV.

TABLE 5
Kinetic Parameters of C-terminally Modified Nanobodies

| | Antigen | | | | | | | | | | | |
|---|---|---|---|---|---|---|---|---|---|---|---|---|
| Nanobody | AAV8 | | | AAV2 | | | AAV9 | | | AAV5 | | |
| Name | kₐ(1/MS) | k_{d}(1/S) | K_{D}(M) | kₐ(1/MS) | k_{d}(1/S) | K_{D}(M) | kₐ(1/MS) | k_{d}(1/S) | K_{D}(M) | kₐ(1/MS) | k_{d}(1/S) | K_{D}(M) |
| A4-T1 | 7.31E+ | 1.48E− | 2.02E− | 5.44E+ | 8.80E− | 1.62E− | 1.68E+ | 1.20E− | 7.13E− | 7.20E+ | 1.93E− | 2.68E− |
| | 03 | 03 | 07 | 03 | 04 | 07 | 05 | 04 | 10 | 03 | 03 | 07 |
| A4-T2 | 7.65E+ | 7.80E− | 1.02E− | 2.55E+ | 6.20E− | 2.43E− | 1.95E+ | 1.00E− | 5.13E− | 6.28E+ | 1.47E− | 2.34E− |
| | 03 | 04 | 07 | 03 | 04 | 07 | 05 | 04 | 10 | 03 | 03 | 07 |
| A4-L1 | 4.23E+ | 9.30E− | 2.20E− | 6.19E+ | 8.70E− | 1.41E− | 1.37E+ | 1.10E− | 8.02E− | 1.62E+ | 9.20E− | 5.69E− |
| | 03 | 04 | 07 | 03 | 04 | 07 | 05 | 04 | 10 | 03 | 04 | 07 |
| A4-M1 | 7.15E+ | 1.40E− | 1.96E− | 4.00E+ | 7.40E− | 1.85E− | 1.83E+ | 1.50E− | 8.21E− | 5.83E+ | 1.89E− | 3.24E− |
| | 03 | 03 | 07 | 03 | 04 | 07 | 05 | 04 | 10 | 03 | 03 | 07 |
| A4-M2 | 5.83E+ | 1.00E− | 1.71E− | 3.22E+ | 3.40E− | 1.06E− | 1.49E+ | 1.30E− | 8.74E− | 4.82E+ | 2.00E− | 4.15E− |
| | 03 | 03 | 07 | 03 | 04 | 07 | 05 | 04 | 10 | 03 | 03 | 07 |

C. AAV Affinity Agents and/or Ligands

### Example 9: Preparation of AAV Affinity Agents and/or Ligands

Activation of agarose gel: 2 g of agarose microspheres were taken, and 2 mol/L NaOH and 0.8 mL of 1,4-butanediol diglycidyl ether were added thereto in the above ratio; the mixture was reacted for at least 60 minutes. After completion of the reaction, the gel was washed with a large amount of deionized water and then suction-filtered to form a wet cake.

Immobilization of AAV nanobodies: The activated agarose gel carrier material was taken, and 2 mL of a nanobody solution was added thereto (in this example, the nanobody solution was prepared in PBS, alternatively, physiological saline or deionized water may be used); the coupling reaction was performed at 37° C. and 250 rpm for 24 hours. After completion of the reaction, ethanolamine (6% by volume, pH 9.0) in an amount of 3 times the volume thereof was added for overnight blocking, thereby obtaining the AAV affinity agents and/or ligands.
\textcircled{1} The nanobody used was A4-002, with a concentration of 10.38 mg/mL, a gel-to-water ratio of 1:3, an epoxy group density on the gel of 3 μmol/g, and a final nanobody coupling amount of 11.58 mg/g gel. This affinity agent and/or ligand is denoted as Affinity Agent A.
\textcircled{2} The nanobody used was A4-008, with a concentration of 6.75 mg/mL, a gel-to-water ratio of 1:5, an epoxy group density on the gel of 3 μmol/g, and a final nanobody coupling amount of 9.77 mg/g gel. This affinity agent and/or ligand is denoted as Affinity Agent B.
\textcircled{3} The nanobody used was A4-008, with a concentration of 6.75 mg/mL, a gel-to-water ratio of 1:7, an epoxy group density on the gel of 3 μmol/g, and a final nanobody coupling amount of 10.74 mg/g gel. This affinity agent and/or ligand is denoted as Affinity Agent C.
\textcircled{4} The nanobody used was A4-014, with a concentration of 3.19 mg/mL, a gel-to-water ratio of 1:8, an epoxy group density on the gel of 2 μmol/g, and a final nanobody coupling amount of 3.12 mg/g gel. This affinity agent and/or ligand is denoted as Affinity Agent D.
\textcircled{5} The nanobody used was A4-024, with a concentration of 5.14 mg/mL, a gel-to-water ratio of 1:6, an epoxy group density on the gel of 2 μmol/g, and a final nanobody coupling amount of 6.36 mg/g gel. This affinity agent and/or ligand is denoted as Affinity Agent E.

### Example 10: Production of AAV

HEK293T cells were cultured and switched to antibiotic-free medium 1 day prior to transfection, followed by incubation for 12-24 h to achieve a cell density of 80%.

### Preparation of EZ-Trans-DNA Complexes:

Tube A: A total of 36 μg of three plasmids (pAAV-hrGFP, pAAV-RC, and pHelper) at a molar ratio of 1:1:1 per 15 mL of cells was supplemented with DMEM to the required volume.
Tube B: 108 μL of EZ Trans transfection reagent was diluted with DMEM.

The contents of both tubes were mixed thoroughly and incubated at room temperature for 10-15 min.

Cell transfection: The prepared transfection mixture was slowly added dropwise to each culture dish at 3 mL/dish, followed by gentle swirling to ensure uniform distribution and subsequent incubation in a cell culture incubator. The medium was replaced 24 h post-transfection, and transfection efficiency was monitored using fluorescence microscopy.

Virus harvesting: Viruses were harvested 48-72 h post-transfection. Cells were detached using a cell scraper, collected into 50-mL centrifuge tubes, and centrifuged at 200×g for 5 min to pellet the cells. The cell pellet was resuspended in PBS and disrupted by sonication.

### Example 11: Evaluation of Adsorption Performance of AAV Affinity Agents

1 mL of the prepared AAV affinity agent was packed into a chromatographic column with a column height of 2 cm. After column packing, the column was first rinsed with an equilibration buffer; subsequently, sample loading was performed, followed by another rinse with the equilibration buffer; elution was then carried out using an elution buffer. The total amount of AAV in the loaded sample, flow-through, equilibration buffer, and eluate was determined to calculate the AAV yield. Herein, VP represents the number of viral capsid particles.

AAV yield=[AAV elution amount (VP)÷AAV binding amount (VP)]×100%
Equilibration buffer: 10 mM Tris-300 mM NaCl, pH 7.6;
Elution buffer: 0.1 M NaAc, 0.5 M NaCl, pH 1.5.

Affinity Agent A exhibited a binding capacity of 9.40×10¹³ VP, an elution amount of 8.46×10¹³ VP, and a yield of 90.0% for AAV2; a binding capacity of 1.17×10¹⁴ VP, an elution amount of 9.76×10¹³ VP, and a yield of 83.2% for AAV5; a binding capacity of 9.20×10¹³ VP, an elution amount of 8.31×10¹³ VP, and a yield of 90.3% for AAV6; a binding capacity of 1.02×10¹⁴ VP, an elution amount of 9.27×10¹³ VP, and a yield of 90.9% for AAV8; and a binding capacity of 1.26×10¹⁴ VP, an elution amount of 1.08×10¹⁴ VP, and a yield of 85.5% for AAV9.

Affinity Agent B exhibited a binding capacity of 9.10×10¹³ VP, an elution amount of 7.85×10¹³ VP, and a yield of 86.2% for AAV2; a binding capacity of 8.56×10¹³ VP, an elution amount of 7.67×10¹³ VP, and a yield of 89.6% for AAV5; a binding capacity of 8.78×10¹³ VP, an elution amount of 7.62×10¹³ VP, and a yield of 86.8% for AAV6; a binding capacity of 9.47×10¹³ VP, an elution amount of 7.95×10¹³ VP, and a yield of 83.9% for AAV8; and a binding capacity of 8.83×10¹³ VP, an elution amount of 7.32×10¹³ VP, and a yield of 82.9% for AAV9.

Affinity Agent C exhibited a binding capacity of 1.21×10¹⁴ VP, an elution amount of 9.42×10¹³ VP, and a yield of 77.9% for AAV2; a binding capacity of 8.95×10¹³ VP, an elution amount of 7.48×10¹³ VP, and a yield of 83.6% for AAV5; a binding capacity of 9.43×10¹³ VP, an elution amount of 7.88×10¹³ VP, and a yield of 83.6% for AAV6; a binding capacity of 1.16×10¹⁴ VP, an elution amount of 9.94×10¹³ VP, and a yield of 85.7% for AAV8; and a binding capacity of 9.17×10¹³ VP, an elution amount of 8.53×10¹³ VP, and a yield of 93.0% for AAV9.

Affinity Agent D exhibited a binding capacity of 4.93×10¹³ VP, an elution amount of 4.37×10¹³ VP, and a yield of 88.6% for AAV2; a binding capacity of 3.22×10¹³ VP, an elution amount of 2.83×10¹³ VP, and a yield of 87.9% for AAV5; a binding capacity of 2.59×10¹³ VP, an elution amount of 2.20×10¹³ VP, and a yield of 84.9% for AAV6; a binding capacity of 3.83×10¹³ VP, an elution amount of 3.51×10¹³ VP, and a yield of 91.6% for AAV8; and a binding capacity of 4.30×10¹³ VP, an elution amount of 3.76×10¹³ VP, and a yield of 87.3% for AAV9.

Affinity Agent E exhibited a binding capacity of 7.65×10¹³ VP, an elution amount of 6.63×10¹³ VP, and a yield of 86.7% for AAV2; a binding capacity of 4.80×10¹³ VP, an elution amount of 4.12×10¹³ VP, and a yield of 85.8% for AAV5; a binding capacity of 6.09×10¹³ VP, an elution amount of 5.48×10¹³ VP, and a yield of 90.0% for AAV6; a binding capacity of 5.17×10¹³ VP, an elution amount of 4.41×10¹³ VP, and a yield of 85.3% for AAV8; and a binding capacity of 6.50×10¹³ VP, an elution amount of 5.97×10¹³ VP, and a yield of 91.8% for AAV9.

In summary, the AAV affinity agents prepared in the present invention exhibit binding capacity to AAV of different serotypes with high yields. They are a new type of broad-spectrum AAV affinity agents and can be used for the purification of AAV of various serotypes.

### Example 11: Preparation of AAV Detection Kit

Nanobody A4-021 was subjected to buffer exchange into PBS (pH 7.4) to a concentration of approximately 10 mg/mL, followed by labeling using an HRP conjugation kit (abcam, Lightning-Link® ab 102890) and thorough dialysis for buffer exchange.

### Direct ELISA Detection Method:

AAV standard samples or test samples were added to a high-hydrophobic 96-well plate, followed by incubation on a horizontal shaker for 2 hours; the plate was then washed with PBS. Blocking was performed using 1%-3% skimmed milk powder, after which the plate was washed with PBS. HRP-conjugated nanobody A4-021 was added, and incubation was conducted on a horizontal shaker for 2 hours, followed by washing the plate with PBS. TMB working solution was added, and the mixture was incubated at room temperature in the dark for 30 minutes; the reaction was terminated by adding 2 M sulfuric acid stop solution, and the OD₄₅₀ value was measured.

### Sandwich ELISA Detection Method:

Capture ligands were coated onto a high-hydrophobic 96-well plate, followed by incubation at 4° C. in the dark overnight; the plate was then washed with PBS. Blocking was performed using 1%-3% skimmed milk powder, after which the plate was washed with PBS. AAV standard samples or test samples were added, and incubation was conducted on a horizontal shaker for 2 hours, followed by washing the plate with PBS. HRP-conjugated nanobody A4-021 was added, with incubation on a horizontal shaker for 2 hours; the plate was then washed with PBS. TMB working solution was added, and the mixture was incubated at room temperature in the dark for 30 minutes, after which the reaction was terminated by adding 2 M sulfuric acid stop solution. The OD₄₅₀ value was measured.

The capture ligands may be nanobody A4-021, other nanobodies of the present invention, anti-AAV antibodies, or other ligands capable of binding to AAV. The capture ligands employed in the present example was A4-040.

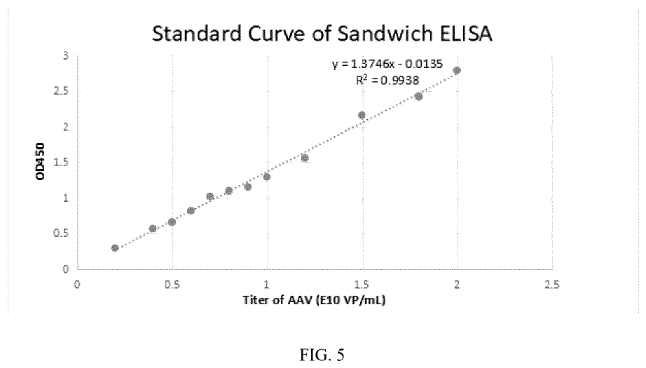

The standard curves of the kit of the present invention for detecting AAV8 are shown in FIGS. 4 and 5, wherein FIG. 4 depicts the standard curve for direct ELISA detection and FIG. 5 depicts the standard curve for sandwich ELISA detection. Both curves exhibit an R²>0.99, indicating a high degree of goodness-of-fit between the experimental data points and the fitted curves. This high R² value demonstrates that the concentration-signal relationships are highly consistent and linear, thereby confirming that the ELISA detection method established based on the nanobodies of the present invention is characterized by high reliability, strong reproducibility, and high sensitivity.

In the prior art, real-time quantitative polymerase chain reaction (qPCR) detection technology is employed for the detection of DNA-containing viral particles; however, this approach is incapable of detecting empty capsids due to its reliance on amplification of viral genomic sequences. In contrast, the present invention enables simultaneous detection of both empty capsids and intact viral particles. The specific detection of empty capsids holds critical significance: not only do empty capsids lack therapeutic efficacy, but they may also potentially elevate immunogenicity and associated adverse reactions. Consequently, the quantitative determination of empty capsids and the control of their content represent a pivotal quality control step in the manufacturing process of AAV-based therapeutics.

The nanobodies of the present invention are anti-AAV nanobodies with novel amino acid sequences identified through screening of phage libraries. These nanobodies and their polypeptides exhibit high affinity and activity, enabling specific recognition and binding to AAV. The affinity agents and/or ligands prepared therefrom possess strong adsorption capacity for AAV and are suitable for AAV purification, while the detection kits prepared therefrom are applicable to AAV detection and quantitative analysis.

The embodiments described above merely represent several implementations of the present invention, and their descriptions are relatively specific and detailed; however, they shall not be construed as limiting the scope of the patent of the present invention. It is contemplated that various combinations or sub-combinations of the specific features and aspects of the disclosed embodiments may be made and still fall within the invention. Further, the disclosure herein of any particular feature, aspect, method, property, characteristic, quality, attribute, element, or the like in connection with an embodiment can be used in all other embodiments set forth herein. Accordingly, various features and aspects of the disclosed embodiments can be combined with or substituted for one another without departing from the spirit of the invention.

It should be noted that, for a person skilled in the art, various modifications and improvements may be made without departing from the inventive concept of the present invention, and all such modifications and improvements shall fall within the protection scope of the present invention. While the invention is susceptible to various modifications and alternative forms, specific examples thereof have been shown and described in detail herein; however, the invention is not to be limited to the particular forms or methods disclosed, but rather covers all modifications, equivalents, and alternatives falling within the spirit and scope of the invention.

Any methods disclosed herein need not be performed in the order recited. The methods disclosed herein include certain actions taken by a practitioner, and also encompass any third-party instruction of those actions, either expressly or by implication.

Thus, the scope of the invention described herein should not be limited by the particular disclosed embodiments described above. The protection scope of the patent of the present invention shall be subject to the appended claims, which include all equivalents of the subject matter of the claims.

### SEQUENCE LISTING XML

A Sequence Listing is submitted herewith as a computer readable form (CRF) in XML format, with a file name of “J-HH001WOUS10-Sequential list.xml”, created on Sep. 2, 2025, and having 162,534 bytes in length. The information in the Sequence Listing is incorporated herein by reference in its entirety.

What is claimed is:

1. A nanobody, characterized in that the variable region of the nanobody's amino acid sequence comprises complementarity-determining regions CDR and framework regions FR; the complementarity-determining regions CDR include a complementarity-determining region CDR1, a complementarity-determining region CDR2, and a complementarity-determining region CDR3; the nanobody is capable of specifically recognizing and binding to AAV; and wherein:
the framework regions FR and the complementarity-determining regions CDR are arranged alternately in sequence; and
the amino acid sequence of the nanobody is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 19, SEQ ID NO: 21, SEQ ID NO: 37, and SEQ ID NO: 107.

2. The nanobody according to claim 1, characterized in that the nanobody is a humanized nanobody, and the humanized nanobody is selected from the group consisting of SEQ ID No: 115 and SEQ ID No: 116.

3. A polypeptide, characterized in that it is obtained by bivalent, trivalent, or tetravalent synthesis of the nanobody according to claim 1.

4. The polypeptide according to claim 3, characterized in that the bivalent polypeptide is a humanized bivalent polypeptide, and the amino acid sequence thereof is selected from the group consisting of SEQ ID NO: 123 and SEQ ID NO: 124.

5. Use of the nanobody according to claim 1 and/or the polypeptide according to claim 3 in the preparation of virus adsorbents, virus purification kits, and virus detection kits, wherein the virus is AAV.

6. A virus affinity agent, characterized in that it comprises a carrier matrix and the nanobody according to claim 1 and/or the polypeptide according to claim 3.


SEQUENCE LISTING (157 sequences)

SEQ ID NO: 1 - protein, 120 aa, synthetic construct
VQLQESGGGLVQPGGSLRLSCAASGRSFSINTMGWYRQAPGKQRELVATVTRGGSTNYAGSVKGRFTISRDNAKNTVYLQMNSLRPEDTAVYYCHIDEVRQSSWTTSNIWGQGTQVTVSS

SEQ ID NO: 2 - protein, 120 aa, synthetic construct
VQLQESGGGLVQPGGSLRLSCAASGRSFSINTMGWYRQAPGKQRELVAAVTRGGSTNYAGSVKGRFTISRDNAKNTVYLQMNSLRPEDTAVYYCHIDEVRQSSWTTSNIWGQGTQVTVSS

SEQ ID NO: 3 - protein, 120 aa, synthetic construct
VQLQESGGGLVQPGGSLRLSCVASGRSFSINTMGWYRQAPGKQRELVAAVTRGGSTNYAGSVKGRFTISRDNAKNTVYLQMNSLRPEDTAVYYCHIDEVRQSSWTTSNIWGQGTQVTVSS

SEQ ID NO: 4 - protein, 120 aa, synthetic construct
VQLQESGGGLVQPGGSLRLSCAASGSRFSINTMGWYRQAPGKQRELVAAVTRGGSTNYAGSVKGRFTISRDNAKNTVYLQMNSLRPEDTAVYYCHIDEVRQSSWTTSNIWGQGTQVTVSS

SEQ ID NO: 5 - protein, 120 aa, synthetic construct
VQLQESGGGLVQAGGSLRLSCAASGRHFSINTMGWYRQAPGKQRELVAAVTRGGGTNYAGSVKGRFTISRDNAKNTVYLQMNSLRPEDTAVYYCHIDEVRQSSWTTSNIWGQGTQVTVSS

SEQ ID NO: 6 - protein, 120 aa, synthetic construct
VQLQESGGGLVQPGGSLRLSCAASGRHFSINTMGWYRQAPGKQRELVAAVTRGGSTNYAGSVKGRFTISRDNAKNTVYLQMNSLRPEDTAVYYCHIDEVRQSSWTTSNIWGQGTQVTVSS

SEQ ID NO: 7 - protein, 120 aa, synthetic construct
VQLQESGGGLVQAGGSLRLSCAASGRHFSINTMGWYRQAPGKQRELVAAVTRGGSTNYAGSVKGRFTISRDNAKNTVYLQMNSLRPEDTAVYYCHIDEVRQSSWTTSNIWGQGTQVTVSS

SEQ ID NO: 8 - protein, 120 aa, synthetic construct
VQLQESGGGLVQAGGSLRLSCAASGRAFSINTMGWYRQAPPKQRELVATVTRGGSTNYAGSVKGRFTISRDNAKNTIYLQMNSLRPEDTAVYYCHVDEVRQSSWTTSNIWGQGTQVTVSS

SEQ ID NO: 9 - protein, 120 aa, synthetic construct
VQLQESGGGLAQPGGSLRLSCAASGSRFSINTMGWYRQAPGKQREFVAAVTRGGSTNYAGSVKGRFTISRDNAKNTVYLQMNTLRPEDTAVYYCHVDEVRQSSWTTSNIWGQGTQVTVSS

SEQ ID NO: 10 - protein, 120 aa, synthetic construct
VQLQESGGGLVQAGGSLRLSCAASGSIFSINSMGWYRQAPGKQRELVATVTRGGNTNYADSVKGRFTISRDNAKNTVYLQMNSLRPEDTAVYYCHVDEVRESSWTTSNLWGQGTQVTVSS

SEQ ID NO: 11 - protein, 120 aa, synthetic construct
VQLQESGGGLVQAGGSLRLSCAASGSIFSINSMGWYRQAPGKQRELVATVTRGGNTNYAGSVKGRFTISRDNAKNTVYLQMNSLRPEDTAVYYCHVDEVRESSWTTSNLWGQGTQVTVSS

SEQ ID NO: 12 - protein, 120 aa, synthetic construct
VQLQESGGGAVQAGGSLRLSCAASGSIFSINSMGWYRQAPGKQRELVATVTRGGNTNYAGSVKGRFTISRDNAKNTVYLQMNSLRPEDTAVYYCHVDEVRESSWTTSNLWGQGTQVTVSS

SEQ ID NO: 13 - protein, 120 aa, synthetic construct
VQLQESGGGLVQPGGSLRLSCAASGSRFSINSMGWYRQAPGKQRELVATVTRGGNTNYADSVKGRFTISRDNAKNTVYLQMNSLRPEDTAVYYCHVDEVRESSWTTSNLWGQGTQVTVSS

SEQ ID NO: 14 - protein, 120 aa, synthetic construct
VQLQESGGGLVQPGGSLRLSCAASGSRFSINSMGWYRQAPGKQRELVATVTRGGNTNYAGSVKGRFTISRDNAKNTVYLQMNSLRPEDTAVYYCHVDEVRESSWTTSNLWGQGTQVTVSS

SEQ ID NO: 15 - protein, 120 aa, synthetic construct
VQLQESGGGLVQPGGSLRLSCAASGSIFSINLMGWYRQAAGKQRELVATITPGGNTNYAGSVKGRFTISRDNAKNTVYLQMNSLRPEDTAVYYCHVDEVRQSSWTTSNLWGQGTQVTVSS

SEQ ID NO: 16 - protein, 120 aa, synthetic construct
VQLQESGGGLVQPGGSLRLSCAASGSIFSINLMGWYRQAPGKQRELVATITPGGNTNYAGSVKGRFTISRDNAKNTVYLQMNSLRPEDTAVYYCHVDEVRQSSWTTSNLWGQGTQVTVSS

SEQ ID NO: 17 - protein, 120 aa, synthetic construct
VQLQESGGGLVQPGGSLRLSCAASGSIFSINLMGWYRQAPGKQRELVATITRGGNTNYAGSVKGRFTISRDNAKNTVYLQMNSLRPEDTAVYYCHVDEVRQSSWTTSNLWGQGTQVTVSS

SEQ ID NO: 18 - protein, 120 aa, synthetic construct
VQLQESGGGLVQAGGSLRLSCAASGSRFSINTMGWYRQAPGKQREFVATVTRGGNTNYAESVKGRFTISRDNAKNTVYLQMNSLRPEDTAVYYCHVDEVRDSSWTTSNIWGQGTQVTVSS

SEQ ID NO: 19 - protein, 120 aa, synthetic construct
VQLQESGGGLVQAGGSLRLSCAASGSTFSINTMGWYRQAPGKQRELVATITRGGSTNYADSVKGRFTISRDNAKNTVYLQMNSLRPEDTAVYYCHVDEVRQSSWTTSNIWGQGTQVTVSS

SEQ ID NO: 20 - protein, 120 aa, synthetic construct
VQLQESGGGLVQAGGSLRLSCAASGSTFSINTMGWYRQAPGKQRELVATITRGGNTNYADSVKGRFTISRDNAKNTVYLQMNSLRPEDTAVYYCHVDEVRQSSWTTSNLWGQGTQVTVSS

SEQ ID NO: 21 - protein, 120 aa, synthetic construct
VQLQESGGGLVQAGGSLRLSCAASGSTFSINTMGWYRQAPGKQRELVATITRGGNTNYADSVKGRFTISRDNAKNTVYLQMNSLRPEDTAVYYCHVDEVRQSSWTTSNIWGQGTQVTVSS

SEQ ID NO: 22 - protein, 120 aa, synthetic construct
VQLQESGGGLVQPGGSLRLSCAASGSGFSINTMGWYRQAPGKQRELVATVTRDGSTNYADFVKGRFTISRDNAKNTVYLQMNSLRPEDTAVYYCHLDEVRESSWTTSNLWGQGTQVTVSS

SEQ ID NO: 23 - protein, 120 aa, synthetic construct
VQLQESGGGLVQPGGSLRLSCAASGSAFSINTMGWYRQAPGKQRELVASVTPGGSTNYADSVKGRFTISRDNAKNTIYLQMNSLRPEDTAVYYCHLDEVRESSWTTSNLWGQGTQVTVSS

SEQ ID NO: 24 - protein, 120 aa, synthetic construct
VQLQESGGGLVQPGGSLRLSCAASGSNFRINTMGWYRQAPGKQRELVATVTRGGSTNYADSVKGRFTISRDNPKNTVYLQMNSLRPEDTAVYYCHVDEVRESSWTTSNLWGQGTQVTVSS

SEQ ID NO: 25 - protein, 120 aa, synthetic construct
VQLQESGGGLVQPGGSLRLSCAASGSNFRINTMGWYRQAPGKQRELVATVTRGGSTNYADSVKGRFTISRDNPKDTVYLQMNSLRPEDTAVYYCHVDEVRESSWTTSNLWGQGTQVTVSS

SEQ ID NO: 26 - protein, 120 aa, synthetic construct
VQLQESGGGLVQPGGSLRLSCAASGSNFRINTMGWYRQAPGKQRELVAPVTRGGSTNYADSVKGRFTISRDNAKNTVYLQMNSLRPEDTAVYYCHVDEVRESSWTTSNLWGQGTQVTVSS

SEQ ID NO: 27 - protein, 120 aa, synthetic construct
VQLQESGGGLVQPGGSLRLSCAASGSNFSINTMGWYRQAPGKQRELVAALTRGGSTNYADSVKGRFTISRDNAKNTVYLQMNSLRPEDTAVYYCHVDEVRESSWTTSNLWGQGTQVTVSS

SEQ ID NO: 28 - protein, 120 aa, synthetic construct
VQLQESGGGLVQPGGSLRLSCAASGSNFSINTMGWYRQAPGKQRELVATVTRGGDTNYADSVKGRFTISRDNAKNTVYLQMNSLRPEDTAVYYCHVDEIRESSWTTSNLWGQGTQVTVSS

SEQ ID NO: 29 - protein, 120 aa, synthetic construct
VQLQESGGGLVQPGGSLRLSCAASGSNFSINTMGWYRQRAGKQRELVATVTRGGSTNYADSVKGRFTISRDNAKNTVYLQMNSLRPEDTAVYYCHIDEIRESSWTTSNLWGQGTQVTVSS

SEQ ID NO: 30 - protein, 120 aa, synthetic construct
VQLQESGGGLVQPGGSLRLSCAASGSSFTINTMGWYRQAPGKQRELVAAVTRGGSTNYAGSVKGRFTISRDNAKNTIYLQMNSLRPEDTAVYYCHVDEIRQSSWTTSNLWGQGTQVTVSS

SEQ ID NO: 31 - protein, 120 aa, synthetic construct
VQLQESGGGLVQPGGSLRLSCAASGSGFSINTMGWYRQAPGKQRELVATVTSAGSTNYAGSVKGRFTISRDNAKNTVYLQMNSLRPEDTAVYYCHVDEIRQSSWTTSNLWGQGTQVTVSS

SEQ ID NO: 32 - protein, 120 aa, synthetic construct
VQLQESGGGLVQPGGSLRLSCVASGSSFSINTMGWYRQAPGKQRELVATVTRDGSTNYANSVKGRFTISRDNAKNTVYLQMNSLRPEDTAVYYCHVDEVRQSSWTTSNLWGQGTQVTVSS

SEQ ID NO: 33 - protein, 120 aa, synthetic construct
VQLQESGGGLVQPGGSLRLSCVASGSSFSINTMGWYRRAPGKQRELVATVTRDGSTNYANSVKGRFTISRDNAKNTVYLQMNSLRPEDTAVYYCHVDEVRQSSWTTSNLWGQGTQVTVSS

SEQ ID NO: 34 - protein, 120 aa, synthetic construct
VQLQESGGGLVQPGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSS

SEQ ID NO: 35 - protein, 120 aa, synthetic construct
VQLQESGGGLVQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSS

SEQ ID NO: 36 - protein, 120 aa, synthetic construct
VQLQESGGGLAQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSS

SEQ ID NO: 37 - protein, 120 aa, synthetic construct
VQLQESGGGLVQPGGSLRLSCAASGSIFSINTMGWYRQAPGKQRELVATVTRGGSTNYAGSVKGRFTISRDNAKNTVYLQMNSLRPEDTAVYYCHLDEVRQSSWTTSNIWGQGTQVTVSS

SEQ ID NO: 38 - protein, 120 aa, synthetic construct
VQLQESGGGLVQPGGSLRLSCAASGSHFSINTMGWYRQAPGKQRELVATVTRGGSTNYANSVKGRFTISRDNAKNTVYLQMNSLRPEDTAVYYCHLDEVRQSSWTTSNLWGQGTQVTVSS

SEQ ID NO: 39 - protein, 120 aa, synthetic construct
VQLQESGGGLVQPGGSLRLSCAASGSGFSINTMGWYRQAPGKWRELVATVTRGGSTNYAGSVKGRFTISRDNAKNTVYLQMNSLRPEDTAVYYCHIDEVRQSSWTTSNLWGQGTQVTVSS

SEQ ID NO: 40 - protein, 120 aa, synthetic construct
VQLQESGGGLVQPGGSLRLSCAASGRIFSINTMGWYRQAPGKQRELVATVTRGGSTNYADSVKGRFTISRDNAKNTVYLQMNSLRPEDTAVYYCHVDEIRQSSWTTSNLWGQGTQVTVSS

SEQ ID NO: 41 - protein, 120 aa, synthetic construct
VQLQESGGGLVQPGGSLRLSCAASGRGFSINTMGWYRQAPGKQRELVATVTRGGSTNYANSVKGRFTISRDNAKNTVYLQMNSLRPEDTAVYYCHVDELRQSSWTTSNLWGQGTQVTVSS

SEQ ID NO: 42 - protein, 120 aa, synthetic construct
VQLQESGGGLVQPGGSLRLSCAASGRAFSINTMGWYRQAPGKQRELVATVTRDGSTNYADSVKGRFTISRDNAKNTVYLQMNSLRPEDTAVYYCHVDEVRQSSWTTSNLWGQGTQVTVSS

SEQ ID NO: 43 - protein, 120 aa, synthetic construct
VQLQESGGGLVQPGGSLRLSCAASGRAFSINTMGWYRQAPGKQRELVATVTRDGSTNYAGSVKGRFTISRDNAKNTVYLQMNSLRPEDTAVYYCHVDEVRQSSWTTSNLWGQGTQVTVSS

SEQ ID NO: 44 - protein, 120 aa, synthetic construct
VQLQESGGGLVQPGGSLRLSCAASGSSFSINTMGWYRQAPGKQRELVATVTRGGVTNYADSVKGRFTISRDNAKNTVYLQMNSLRPEDTAVYYCHVDEVRQSSWTTSNLWGQGTQVTVSS

SEQ ID NO: 45 - protein, 120 aa, synthetic construct
VQLQESGGGLVQPGGSLRLSCAASGSSFSINTMGWYRQAPGKQRELVAAVTRGGVTNYADSVKGRFTISRDNAKNTVYLQMNSLRPEDTAVYYCHVDEVRQSSWTTSNLWGQGTQVTVSS

SEQ ID NO: 46 - protein, 120 aa, synthetic construct
VQLQESGGGLVQPGGSLRLSCAASGSRFSINTMGWYRQAPGKQRELVATVTRGGSTNYADSVKGRFTISRDNAKNTVYLQMNSLRPEDTAVYYCHVDEVRQSSWTTSNLWGQGTQVTVSS

SEQ ID NO: 47 - protein, 120 aa, synthetic construct
VQLQESGGGLVQPGGSLRLSCAASGSRFSINTMGWYRQAPGKQRELVATVTRGGSTNYAGSVKGRFTISRDNAKNTVYLQMNSLRPEDTAVYYCHVDEVRQSSWTTSNLWGQGTQVTVSS

SEQ ID NO: 48 - protein, 120 aa, synthetic construct
VQLQESGGGLVQPGGSLRLSCAASGSMFSINTMGWYRQAPGKQRELVATVTRGGNTNYAGSVKGRFTISRDNAKNTVYLQMNSLRPEDTAVYYCHVDEVRQSSWTTSNLWGQGTQVTVSS

SEQ ID NO: 49 - protein, 120 aa, synthetic construct
VQLQESGGGLVQPGGSLRLSCAASGSMFSINTMGWYRQAPGKQRELVAAVTRGGNTNYAGSVKGRFTISRDNAKNTVYLQMNSLRPEDTAVYYCHVDEVRQSSWTTSNLWGQGTQVTVSS

SEQ ID NO: 50 - protein, 120 aa, synthetic construct
VQLQESGGGLVQPGGSLRLSCAASGSSFVINTMGWYRQAPGKQRELVAAVTRGGSTNYADSVKGRFTISRDNAKNTVYLQMNSLRPEDTAVYYCHVDEVRQSSWTTSNLWGQGTQVTVSS

SEQ ID NO: 51 - protein, 120 aa, synthetic construct
VQLQESGGGLVQPGGSLRLSCAASGSSFVINTMGWYRRAPGKQRELVAAVTRGGSTNYADSVKGRFTISRDNAKNTVYLQMNSLRPEDTAVYYCHVDEVRQSSWTTSNLWGQGTQVTVSS

SEQ ID NO: 52 - protein, 120 aa, synthetic construct
VQLQESGGGLVQPGGSLRLSCAASGSIFIINTMGWYRQAPGKQRELVAAVTRGGSTNYADSVKGRFTISRDNAKNTVYLQMNSLRPEDTAVYYCHVDEVRQSSWTTSNLWGQGTQVTVSS

SEQ ID NO: 53 - protein, 120 aa, synthetic construct
VQLQESGGGLVQPGGSLRLSCAASGSIFIINTMGWYRRAPGKQRELVAAVTRGGSTNYADSVKGRFTISRDNAKNTVYLQMNSLRPEDTAVYYCHVDEVRQSSWTTSNLWGQGTQVTVSS

SEQ ID NO: 54 - protein, 120 aa, synthetic construct
VQLQESGGGLVQPGGSLRLSCAASGSAFSINTMGWYRQAPGKQRELVAAVTRGGSTNYADSVKGRFTISRDNAKNTVYLQMNSLRPEDTAVYYCHVDEVRQSSWTTSNLWGQGTQVTVSS

SEQ ID NO: 55 - protein, 120 aa, synthetic construct
VQLQESGGGLVQPGGSLRLSCAASGSIFSINTMGWYRQAPGKQRELVAAVTRGGSTNYADSVKGRFTISRDNAKNTVYLQMNSLRPEDTAVYYCHVDEVRQSSWTTSNLWGQGTQVTVSS

SEQ ID NO: 56 - protein, 120 aa, synthetic construct
VQLQESGGGLVQPGGSLRLSCAASGSAFSINTMGWYRQAPGKQREKVAAVTRGGSTNYADSVKGRFTISRDNAKNTVYLQMNSLRPEDTAVYYCHVDEVRQSSWTTSNLWGQGTQVTVSS

SEQ ID NO: 57 - protein, 120 aa, synthetic construct
VQLQESGGGLVQPGGSLRLSCAASGSIFSINTMGWYRQAPGKQREKVAAVTRGGSTNYADSVKGRFTISRDNAKNTVYLQMNSLRPEDTAVYYCHVDEVRQSSWTTSNLWGQGTQVTVSS

SEQ ID NO: 58 - protein, 120 aa, synthetic construct
VQLQESGGGLVQPGGSLRLSCAASGSIFSINTMGWYRQAPAKQREIVATVTRDGSTNYAESVKGRFTISRDNAKNTVYLQMNSLRPEDTAVYYCHLDEVRQSTWTTSNLWGQGTQVTVSS

SEQ ID NO: 59 - protein, 120 aa, synthetic construct
VQLQESGGGAVQPGGSLRLSCAASGSHFSLNTMGWYRRAPGKQRELVATVTRGGSTNYADSVKGRFTISRDNAKNTIYLQMNSLRPEDTAVYYCHVDEVRQSTWTTSNLWGQGTQVTVSS

SEQ ID NO: 60 - protein, 120 aa, synthetic construct
VQLQESGGGLVQPGGSLRLSCAASGSTFSLNTMGWYRQAPGKQREIVATVTRGGSTNYAESVKGRFTISRDNAKNTVYLQMNSLRPEDTAVYYCHVDEVRQSTWTTSNLWGQGTQVTVSS

SEQ ID NO: 61 - protein, 120 aa, synthetic construct
VQLQESGGGLVQPGGSLRLSCAASGSRFSLNTMGWYRQAPGKQRELVATVTRGGSTNYANSVKGRFTISRDNAKNTVYLQMNSLRPEDTAVYYCHVDEVRQSTWTTSNLWGQGTQVTVSS

SEQ ID NO: 62 - protein, 120 aa, synthetic construct
VQLQESGGGLVQAGGSLRLSCAASGSSFSINAMGWYRQAPGKQRELVATVTRGGTTNYADSVKGRFTISRDNPKNTVYLQMNSLRPEDTAVYYCHLDEVRESSWTTSNLWGQGTQVTVSS

SEQ ID NO: 63 - protein, 120 aa, synthetic construct
VQLQESGGGLVQPGGSLRLSCAASGRRFTINTMGWYRRAPGKQRELVATITRGGSTNYAGSVKGRFTISRDNPKNTVYLQMNSLRPEDTAVYYCHIDEVRQSSWTTSNLWGQGTQVTVSS

SEQ ID NO: 64 - protein, 120 aa, synthetic construct
VQLQESGGGLVQSGGSLRLSCAASGRNFSLNTVGWYRQAPGKQRELVATVTSGGGTNYAGSVKGRFTISRDNAKNTVYLQMNSLRPEDTAVYYCHVDEVRESTWTTSNLWGQGTQVTVSS

SEQ ID NO: 65 - protein, 120 aa, synthetic construct
VQLQESGGGLVQSGGSLRLSCAASGRNFSLNTVGWYRQAPGKQRELVATVTRGGSTNYAGSVKGRFTISRDNAKNTVYLQMNSLRPEDTAVYYCHVDEVRESSWTTSNLWGQGTQVTVSS

SEQ ID NO: 66 - protein, 120 aa, synthetic construct
VQLQESGGGLVQTGGSLRLSCAASGRAFSLNTMGWYRQAPGKQRELVATVTRGGSTNYAGSVKGRFTISRDNAKNTVYLQMNSLRPEDTAVYYCHVDEVRESSWTTSNLWGQGTQVTVSS

SEQ ID NO: 67 - protein, 120 aa, synthetic construct
VQLQESGGGLVQTGGSLRLSCAASGRAFSLNTMGWYRQAPGKWRELVATVTRGGSTNYAGSVKGRFTISRDNTKNTVYLQMNNLRPEDTAVYYCHVDEVRESSWTTSNLWGQGTQVTVSS

SEQ ID NO: 68 - protein, 120 aa, synthetic construct
VQLQESGGGLVQTGGSLRLSCAASGRAFSLNTMGWYRQAPGKWRELVATVTRGGSTNYAGSVKGRFTISRDNAKNTVYLQMNNLRPEDTAVYYCHVDEVRESSWTTSNLWGQGTQVTVSS

SEQ ID NO: 69 - protein, 120 aa, synthetic construct
VQLQESGGGLVQPGGSLRLSCAASGSIFTINSMGWYRQAPGKWRELVATVTRGGNTNYANSVKGRFTISRDNAKNTVYLQMNTLRPEDTAVYYCHLDEVRDSSWTTSNLWGQGTQVTVSS

SEQ ID NO: 70 - protein, 120 aa, synthetic construct
VQLQESGGGLVQPGGSLRLSCAASGSIFSINSMGWYRQAPGKWRELVATVTRGGSTNYANSVKGRFTISRDNAKNTVYLQMNTLRPEDTAVYYCHLDEVRDSSWTTSNLWGQGTQVTVSS

SEQ ID NO: 71 - protein, 120 aa, synthetic construct
VQLQESGGGAVQAGGSLRLSCAASGSTFSINTMGWYRQAPGKQRELVATVTRGGSTNYGDFVKGRFTISRDNAKNTVYLQMNTLRPEDTAVYYCHLDEVRQSSWTTSNLWGQGTQVTVSS

SEQ ID NO: 72 - protein, 120 aa, synthetic construct
VQLQESGGGLAQPGGSLRLSCAASGSSFVINTMGWYRQAPGKQREFVATVTRGGSTNYGEFVKGRFTISRDNAKNTVYLQMNSLRPEDTAVYYCHVDELRQSSWTTSNLWGQGTQVTVSS

SEQ ID NO: 73 - protein, 120 aa, synthetic construct
VQLQESGGGLVQPGGSLRLSCAASGSNFSINTMGWYRQAPGKQREMVATVTRGGSTNYGDFVKGRFTISRDNAKNTVYLQMNSLRPEDTAVYYCHVDEIRQSSWTTSNIWGQGTQVTVSS

SEQ ID NO: 74 - protein, 120 aa, synthetic construct
VQLQESGGGLVQSGGSLRLSCAASGSMFSLNTMGWYRQAPGKQRELVATITRGGGTSYADSVKGRFTISRDNAKNTVYLQMNSLRPEDTAVYYCHVDELRQSSWTTSNLWGQGTQVTVSS

SEQ ID NO: 75 - protein, 120 aa, synthetic construct
VQLQESGGGLVQSGGSLRLSCAASGSMFSLNTMGWYRQAPGKQRELVATITRGGGTNYADSVKGRFTISRDNAKNTVYLQMNSLRPEDTAVYYCHVDELRQSSWTTSNLWGQGTQVTVSS

SEQ ID NO: 76 - protein, 120 aa, synthetic construct
VQLQESGGGLVHSGGSLRLSCAASGSMFSLNTMGWYRQAPGKQRELVATITRGGGTSYADSVKGRFTISRDNAKNTVYLQMNSLRPEDTAVYYCHVDELRQSSWTTSNLWGQGTQVTVSS

SEQ ID NO: 77 - protein, 120 aa, synthetic construct
VQLQESGGGLVQPGGSLRLSCAASGSMFSINIMGWYRQAPGKQREFVATITRGGSTNYADFVKGRFTISRDNAKNTVYLQMNSLRPEDTAVYYCHVDELRDSSWTTSNLWGQGTQVTVSS

SEQ ID NO: 78 - protein, 120 aa, synthetic construct
VQLQESGGGLVQPGGSLRLSCLASGTMFSLNTMGWYRQAPGKQRELVATVTRGGSTNYADFVKGRFTISRDNAKNTVYLQMNSLRPEDTAVYYCHVDEVRDSSWTTSNLWGQGTQVTVSS

SEQ ID NO: 79 - protein, 120 aa, synthetic construct
VQLQESGGGLVQPGGSLRLSCAASGTRFSINTMGWYRQAPGKQRELVANVTPAGSTNYADSVKGRFTISRDNAKNTVYLQMNSLRPEDTAVYYCHVDELRDSSWTTSNLWGQGTQVTVSS

SEQ ID NO: 80 - protein, 120 aa, synthetic construct
VQLQESGGGLVQPGGSLRLSCAASGSRFSLNTMGWYRQAPGKQRELVATVTRGGSTNYADFVKGRFTISRDNAKNTVYLQMNSLRPEDTAVYYCHVDELRESGWTTSNLWGQGTQVTVSS

SEQ ID NO: 81 - protein, 120 aa, synthetic construct
VQLQESGGGLVQPGGSLRLSCAASGSRFSLNTMGWYRQAPGKQRELVATVTRGGSTNYADSVKGRFTISRDNAKNTVYLQMNSLRPEDTAVYYCHVDELRESGWTTSNLWGQGTQVTVSS

SEQ ID NO: 82 - protein, 120 aa, synthetic construct
VQLQESGGGLVQPGGSLRLSCAASGSRFSLNTMGWYRQAPGKQRELVATVTRGGSTKYADFVKGRFTISRDNAKNTVYLQMNSLRPEDTAVYYCHVDELRESGWTTSNLWGQGTQVTVSS

SEQ ID NO: 83 - protein, 120 aa, synthetic construct
VQLQESGGGLVQTGGSLKLSCAASGSRFSLNTMGWYRQAPRKQREFVAAVTRGGNTNYGDFVKGRFTISRDNAKNTVYLQMNSLRPEDTAVYYCHVDELRESSWTTSNLWGQGTQVTVSS

SEQ ID NO: 84 - protein, 120 aa, synthetic construct
VQLQESGGGLVQPGGSLKLSCAASGSRFSLNTMGWYRQAPRKQREFVAAVTRGGNTNYGDFVKGRFTISRDNAKNTVYLQMNSLRPEDTAVYYCHVDELRESSWTTSNLWGQGTQVTVSS

SEQ ID NO: 85 - protein, 120 aa, synthetic construct
VQLQESGGGLVQPGGSLRLSCAASGSRFSLNTMGWYRQAPGKQREFVAAVTRGGNTNYGDFVKGRFTISRDNAKNTVYLQMNSLRPEDTAVYYCHVDELRESSWTTSNLWGQGTQVTVSS

SEQ ID NO: 86 - protein, 120 aa, synthetic construct
VQLQESGGGLVQPGGSLRLSCAASGSRFSLNTMGWYRQAPGKQRELVAAVTRGGNTNYADFVKGRFTISRDNAKNTVYLQMNSLRPEDTAVYYCHVDELRESSWTTSNLWGQGTQVTVSS

SEQ ID NO: 87 - protein, 120 aa, synthetic construct
VQLQESGGGLVQPGGSLRLSCAASGSRFSLNTMGWYRQAPGKQRELVAAVTRGGNTNYGDFVKGRFTISRDNAKNTVYLQMNSLRPEDTAVYYCHVDELRESSWTTSNLWGQGTQVTVSS

SEQ ID NO: 88 - protein, 120 aa, synthetic construct
VQLQESGGGLVQTGGSLRLSCAASGSRFSLNTMGWYRQAPGKQRELVAAVTRGGNTNYGDFVKGRFTISRDNAKNTVYLQMNSLRPEDTAVYYCHVDELRESSWTTSNLWGQGTQVTVSS

SEQ ID NO: 89 - protein, 120 aa, synthetic construct
VQLQESGGGLVQPGGSLRLSCAASGSNFSINTMGWYRQAPGKQRELVATFTRAGNTNYADSVKGRFTISRDNGKNTVYLQMNSLRPEDTAVYYCHVDEVRDSSWTTSNLWGQGTQVTVSS

SEQ ID NO: 90 - protein, 120 aa, synthetic construct
VQLQESGGGLVQPGGSLRLSCVASGSRFIINTMGWYRQAPGKQRELVATFTRGGNTNYAESVKGRFTISRDNGKSTVYLQMNSLRPEDTAVYYCHVDEVRESTWTTSNLWGQGTQVTVSS

SEQ ID NO: 91 - protein, 120 aa, synthetic construct
VQLQESGGGLVQPGGSLRLSCAASGSRFSINTMGWYRQRAGKQRELVATVTRDGSTTYANSVKGRFTISRDNAKNTVYLQMNSLRPEDTAVYYCHVDELRQSTWTTSNLWGQGTQVTVSS

SEQ ID NO: 92 - protein, 120 aa, synthetic construct
VQLQESGGGLVQTGGSLRLSCLASGSIFSINTMGWYRQRAGKQRELVATVTRAGSTNYADSVKGRFTISRDNAKNTVYLQMNSLRPEDTAVYYCHVDEIRESSWTTSNLWGQGTQVTVSS

SEQ ID NO: 93 - protein, 120 aa, synthetic construct
VQLQESGGGLVQTGGSLRLSCAASGSIFIINTMGWYRQRAGKQRELVATVTRGGSTNYADSVKGRFTISRDNAKNTVYLQMNTLRPEDTAVYYCHVDELRQSSWTTSNLWGQGTQVTVSS

SEQ ID NO: 94 - protein, 120 aa, synthetic construct
VQLQESGGGLVQPGGSLRLSCVASGSIFVINTMGWYRQAPGKQRELVATVTRDGGTNYADSVKGRFTISRDNAKNTVYLQMNSLRPEDTAVYYCHIDEIRESSWTTSNLWGQGTQVTVSS

SEQ ID NO: 95 - protein, 120 aa, synthetic construct
VQLQESGGGLVQPGGSLRLSCAASGSSFSANTMGWYRQAPGKQRELVATITRGGNTNYGDSVKGRFTISRDNAKDTVYLQMNSLRPEDTAVYYCHIDEIRESTWTTSNLWGQGTQVTVSS

SEQ ID NO: 96 - protein, 120 aa, synthetic construct
VQLQESGGGLVQPGGSLRLSCIASGSSFSANTMGWYRQAPGKQRELVATITRGGNTNYGDSVKGRFTISRDNAKNTVYLQMNSLRPEDTAVYYCHIDEIRESTWTTSNLWGQGTQVTVSS

SEQ ID NO: 97 - protein, 120 aa, synthetic construct
VQLQESGGGLVQPGGSLRLSCIASGSIFSANTMGWYRQAPGKQRELVATITRGGNTNYGDSVKGRFTISRDNAKNTVYLQMNTLRPEDTAVYYCHVDEIRESSWTTSNLWGQGTQVTVSS

SEQ ID NO: 98 - protein, 120 aa, synthetic construct
VQLQESGGGLVQPGGSLRLSCIASGSIFSINTMGWYRQAPGKQRELVATITRGGNTNYGDSVKGRFTISRDNAKNTVYLQMNTLRPEDTAVYYCHVDEIRESSWTTSNLWGQGTQVTVSS

SEQ ID NO: 99 - protein, 120 aa, synthetic construct
VQLQESGGGLVQTGGSLRLSCAASGSSFSINTMGWYRQAPGKWRELVAAVTRGGDTNYAESVKGRFTISRDNGKNTVYLQMNSLRPEDTAVYYCHIDEIRESSWTTSNLWGQGTQVTVSS

SEQ ID NO: 100 - protein, 120 aa, synthetic construct
VQLQESGGGLVQPGGSLRLSCAASGSIFVINTMGWYRQAPGKQRELVASLTRGGSTNYADSVKGRFTISRDNDKNTIYLQMNSLRPEDTAVYYCHVDEVRESTWTTSNIWGQGTQVTVSS

SEQ ID NO: 101 - protein, 120 aa, synthetic construct
VQLQESGGGLVQPGGSLRLSCAASGSRFSINIMGWYRQAPGKQRELVASVTRGGTTNYADSVKGRFTISRDNPKNTIYLQMNSLRPEDTAVYYCHVDEIRESTWTTSNLWGQGTQVTVSS

SEQ ID NO: 102 - protein, 120 aa, synthetic construct
VQLQESGGGLVQPGGSLRLSCAASGSHFTINSVGWYRQAPGKQRELVAPVTRGGTTNYADFVKGRFTISRDNAKNTVYLQMNSLRPEDTAVYYCHVDELRDSSWTTSNLWGQGTQVTVSS

SEQ ID NO: 103 - protein, 120 aa, synthetic construct
VQLQESGGGLVQPGGSLRLSCAASGSIFSLNAMGWYRQAPGKQRELVATITRGGSTNYADSVKGRFTISRDNAKNTVYLQMNSLRPEDTAVYYCHVDEVRESIWTTSNLWGQGTQVTVSS

SEQ ID NO: 104 - protein, 120 aa, synthetic construct
VQLQESGGGLVQPGGSLRLSCAASGSIFSLNSMGWYRQAPGKQRELVATITSGGSTNYAGSVKGRFTISRDNAKNTVYLQMNNLQPEDTAVYYCHVDEVRESSWTTSNLWGQGTQVTVSS

SEQ ID NO: 105 - protein, 120 aa, synthetic construct
VQLQESGGGLVQPGGSLRLSCAASGSIFSLNAMGWYRQAPGKQRELVATITSGGSTNYADSVKGRFTISRDNAKNTAYLQMNNLQPEDTAVYYCHVDEVRESIWTTSNLWGQGTQVTVSS

SEQ ID NO: 106 - protein, 120 aa, synthetic construct
VQLQESGGGLVQPGGSLRLSCIASGSIFSLNAMGWYRQAPGKQRELVATITSGGSTNYADSVKGRFTISRDNAKNTAYLQMNNLQPEDTAVYYCHVDEVRESIWTTSNLWGQGTQVTVSS

SEQ ID NO: 107 - protein, 120 aa, synthetic construct
VQLQESGGGLVQPGGSLRLSCIASGSGFSINTVGWYRQAPAKQRELVATLTRGGSTNYADSVKGRFTISRDNPKNTVYLQMNSLRPEDTAVYYCHVDEVRQSSWTTSNIWGQGTQVTVSS

SEQ ID NO: 108 - protein, 120 aa, synthetic construct
VQLQESGGGLAQPGGSLRLSCLASGSGFSINTMGWYRQAPPKWRELVATVTRGGSTNYAGSVKGRFTISRDNPKNTVYLQMNSLRPEDTAVYYCHLDEVRQSTWTTSNIWGQGTQVTVSS

SEQ ID NO: 109 - protein, 120 aa, synthetic construct
VQLQESGGGLVQAGGSLRLSCAASGTIFSLNVMGWYRQAPGKQRELVASVTRGGNTNYADSVKGRFTISRDNAKTTVYLQMNSLRPEDTAVYYCHVDELRDSTWTTSNLWGQGTQVTVSS

SEQ ID NO: 110 - protein, 120 aa, synthetic construct
VQLQESGGGLVQPGGSLRLSCFASGTNFSINVVGWYRQAPGKQRELVATITRGGSTNYAESVKGRFTISRDNAKNTVYLQMNNLRPEDTAVYYCHLDEVRDSSWTTSNLWGQGTQVTVSS

SEQ ID NO: 111 - protein, 120 aa, synthetic construct
VQLQESGGGLAQPGGSLRLSCAASGSNFSINTMGWYRQRAGKQREFVATITRGGSTNYGDSVKGRFTISRDNAKTTVYLQMNSLRPEDTAVYYCHLDEVRESTWTTSNLWGQGTQVTVSS

SEQ ID NO: 112 - protein, 120 aa, synthetic construct
VQLQESGGGLVQTGGSLRLSCIASGSIFSINTVGWYRQAPPKQRELVATVTRAGSTWYAGSVKGRFTISRDNAKNTIYLQMNTLRPEDTAVYYCHVDEVRQSTWTTSNIWGQGTQVTVSS

SEQ ID NO: 113 - protein, 120 aa, synthetic construct
VQLQESGGGLVQTGGSLRLSCVASGSRFSINTVGWYRQAPGKQRELVATVTRGGSTWYAGSVKGRFTISRDNTKNTIYLQMNSLRPEDTAVYYCHVDEIRQSTWTTSNLWGQGTQVTVSS

SEQ ID NO: 114 - protein, 120 aa, synthetic construct
VQLQESGGGLVQTGGSLRLSCVASGSIFSINTVGWYRQAPGKQRELVATVTRAGSTWYAGSVKGRFTISRDNTKNTIYLQMNSLRPEDTAVYYCHVDELRQSTWTTSNLWGQGTQVTVSS

SEQ ID NO: 115 - protein, 120 aa, synthetic construct
VQLVESGGGLVQPGGSLRLSCAASGRSFSINTMGWFRQAPGKGLEAVAIVTRGGSTYYADSVKGRFTISRDNSKNTLYLQMNSLRAEDTAVYYCHIDEVRQSSWTTSNIWGQGTLVTVSS

SEQ ID NO: 116 - protein, 120 aa, synthetic construct
VQLQASGGGFVQPGGSLRLSCAASGRSFSINTMGWFRQAPGKEREFVAVVTRGGSTYYADSVKGRFTISRDNSKNTVYLQMNSLRAEDTATYYCHIDEVRQSSWTTSNIWGQGTQVTVSS

SEQ ID NO: 117 - protein, 120 aa, synthetic construct
VQLQASGGGFVQPGGSLRLSCAASGSIFSINLMGWFRQAPGKERELVAVITPGGNTYYADSVKGRFTISRDNSKNTVYLQMNSLRAEDTATYYCHVDEVRQSSWTTSNLWGQGTQVTVSS

SEQ ID NO: 118 - protein, 120 aa, synthetic construct
VQLVESGGGLVQPGGSLRLSCAASGSIFSINLMGWFRQAPGKGLELVAIITPGGNTYYADSVKGRFTISRDNSKNTVYLQMNSLRAEDTAVYYCHVDEVRQSSWTTSNLWGQGTLVTVSS

SEQ ID NO: 119 - protein, 120 aa, synthetic construct
VQLQASGGGFVQPGGSLRLSCAASGRGFSINTMGWFRQAPGKERELVAVVTRGGTTYYADSVKGRFTISRDNSKNTVYLQMNSLRAEDTAVYYCHVDEVRQSSWTTSNLWGQGTQVTVSS

SEQ ID NO: 120 - protein, 120 aa, synthetic construct
VQLVESGGGLVQPGGSLRLSCAASGRGFSINTMGWFRQAPGKGRELVAIVTRGGTTYYADSVKGRFTISRDNSKNTVYLQMNSLRAEDTAVYYCHVDEVRQSSWTTSNLWGQGTLVTVSS

SEQ ID NO: 121 - protein, 255 aa, synthetic construct
VQLQESGGGLVQPGGSLRLSCAASGSIFSINLMGWYRQAPGKQRELVATITRGGNTNYAGSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCAASGSIFSINLMGWYRQAPGKQRELVATITRGGNTNYAGSVKGRFTISRDNAKNTVYLQMNSLRPEDTAVYYCHVDEVRQSSWTTSNLWGQGTQVTVSS

SEQ ID NO: 122 - protein, 255 aa, synthetic construct
VQLQESGGGLVQAGGSLRLSCAASGSRFSINTMGWYRQAPGKQREFVATVTRGGNTNYAESVKGRFTISRDNAKNTVYLQMNSLRPEDTAVYYCHVDEVRDSSWTTSNIWGQGTQVTVSSGGGGSGGGGSGGGGSVQLQESGGGLVQAGGSLRLSCAASGSTFSINTMGWYRQAPGKQRELVATITRGGNTNYADSVKGRFTISRDNAKNTVYLQMNSLRPEDTAVYYCHVDEVRQSSWTTSNLWGQGTQVTVSS

SEQ ID NO: 123 - protein, 255 aa, synthetic construct
VQLVESGGGLVQPGGSLRLSCAASGRSFSINTMGWFRQAPGKGLEAVAIVTRGGSTYYADSVKGRFTISRDNSKNTLYLQMNSLRAEDTAVYYCHIDEVRQSSWTTSNIWGQGTLVTVSSGGGGSGGGGSGGGGSVQLVESGGGLVQPGGSLRLSCAASGRSFSINTMGWFRQAPGKGLEAVAIVTRGGSTYYADSVKGRFTISRDNSKNTLYLQMNSLRAEDTAVYYCHIDEVRQSSWTTSNIWGQGTLVTVSS

SEQ ID NO: 124 - protein, 255 aa, synthetic construct
VQLVESGGGLVQPGGSLRLSCAASGRSFSINTMGWFRQAPGKGLEAVAIVTRGGSTYYADSVKGRFTISRDNSKNTLYLQMNSLRAEDTAVYYCHIDEVRQSSWTTSNIWGQGTLVTVSSGGGGSGGGGSGGGGSVQLVESGGGLVQPGGSLRLSCAASGSIFSINLMGWFRQAPGKGLELVAIITPGGNTYYADSVKGRFTISRDNSKNTVYLQMNSLRAEDTAVYYCHVDEVRQSSWTTSNLWGQGTLVTVSS

SEQ ID NO: 125 - protein, 390 aa, synthetic construct
VQLQESGGGLVQPGGSLRLSCAASGSGFSINTMGWYRQAPGKQRELVATVTRDGSTNYADFVKGRFTISRDNAKNTVYLQMNSLRPEDTAVYYCHLDEVRESSWTTSNLWGQGTQVTVSSGGGGSGGGGSGGGGSVQLQESGGGLVQPGGSLRLSCAASGSGFSINTMGWYRQAPGKQRELVATVTRDGSTNYADFVKGRFTISRDNAKNTVYLQMNSLRPEDTAVYYCHLDEVRESSWTTSNLWGQGTQVTVSSGGGGSGGGGSGGGGSVQLQESGGGLVQPGGSLRLSCAASGSGFSINTMGWYRQAPGKQRELVATVTRDGSTNYADFVKGRFTISRDNAKNTVYLQMNSLRPEDTAVYYCHLDEVRESSWTTSNLWGQGTQVTVSS

SEQ ID NO: 126 - protein, 390 aa, synthetic construct
VQLQESGGGLVQPGGSLRLSCAASGSAFSINTMGWYRQAPGKQRELVASVTPGGSTNYADSVKGRFTISRDNAKNTIYLQMNSLRPEDTAVYYCHLDEVRESSWTTSNLWGQGTQVTVSSGGGGSGGGGSGGGGSVQLQESGGGLVQPGGSLRLSCAASGSAFSINTMGWYRQAPGKQRELVASVTPGGSTNYADSVKGRFTISRDNAKNTIYLQMNSLRPEDTAVYYCHLDEVRESSWTTSNLWGQGTQVTVSSGGGGSGGGGSGGGGSVQLQESGGGLVQPGGSLRLSCAASGSAFSINTMGWYRQAPGKQRELVASVTPGGSTNYADSVKGRFTISRDNAKNTIYLQMNSLRPEDTAVYYCHLDEVRESSWTTSNLWGQGTQVTVSS

SEQ ID NO: 127 - protein, 390 aa, synthetic construct
VQLQESGGGLVQPGGSLRLSCAASGSIFSINLMGWYRQAPGKQRELVATITRGGNTNYAGSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCAASGSIFSINLMGWYRQAPGKQRELVATITRGGNTNYAGSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCAASGSNFRINTMGWYRQAPGKQRELVATVTRGGSTNYADSVKGRFTISRDNPKDTVYLQMNSLRPEDTAVYYCHVDEVRESSWTTSNLWGQGTQVTVSS

SEQ ID NO: 128 - protein, 390 aa, synthetic construct
VQLQESGGGLVQAGGSLRLSCAASGSRFSINTMGWYRQAPGKQREFVATVTRGGNTNYAESVKGRFTISRDNAKNTVYLQMNSLRPEDTAVYYCHVDEVRDSSWTTSNIWGQGTQVTVSSGGGGSGGGGSGGGGSVQLQESGGGLVQAGGSLRLSCAASGSRFSINTMGWYRQAPGKQREFVATVTRGGNTNYAESVKGRFTISRDNAKNTVYLQMNSLRPEDTAVYYCHVDEVRDSSWTTSNIWGQGTQVTVSSGGGGSGGGGSGGGGSVQLQESGGGLVQPGGSLRLSCAASGSNFRINTMGWYRQAPGKQRELVATVTRGGSTNYADSVKGRFTISRDNPKDTVYLQMNSLRPEDTAVYYCHVDEVRESSWTTSNLWGQGTQVTVSS

SEQ ID NO: 129 - protein, 390 aa, synthetic construct
VQLQESGGGLVQPGGSLRLSCAASGSNFRINTMGWYRQAPGKQRELVATVTRGGSTNYADSVKGRFTISRDNPKNTVYLQMNSLRPEDTAVYYCHVDEVRESSWTTSNLWGQGTQVTVSSGGGGSGGGGSGGGGSVQLQESGGGLVQPGGSLRLSCAASGSNFSINTMGWYRQAPGKQRELVATVTRGGDTNYADSVKGRFTISRDNAKNTVYLQMNSLRPEDTAVYYCHVDEIRESSWTTSNLWGQGTQVTVSSGGGGSGGGGSGGGGSVQLQESGGGLVQPGGSLRLSCAASGSNFRINTMGWYRQAPGKQRELVATVTRGGSTNYADSVKGRFTISRDNPKNTVYLQMNSLRPEDTAVYYCHVDEVRESSWTTSNLWGQGTQVTVSS

SEQ ID NO: 130 - protein, 390 aa, synthetic construct
VQLQESGGGLVQPGGSLRLSCAASGSNFRINTMGWYRQAPGKQRELVAPVTRGGSTNYADSVKGRFTISRDNAKNTVYLQMNSLRPEDTAVYYCHVDEVRESSWTTSNLWGQGTQVTVSSGGGGSGGGGSGGGGSVQLQESGGGLVQPGGSLRLSCAASGSNFSINTMGWYRQAPGKQRELVAALTRGGSTNYADSVKGRFTISRDNAKNTVYLQMNSLRPEDTAVYYCHVDEVRESSWTTSNLWGQGTQVTVSSGGGGSGGGGSGGGGSVQLQESGGGLVQPGGSLRLSCAASGSNFSINTMGWYRQAPGKQRELVATVTRGGDTNYADSVKGRFTISRDNAKNTVYLQMNSLRPEDTAVYYCHVDEIRESSWTTSNLWGQGTQVTVSS

SEQ ID NO: 131 - protein, 390 aa, synthetic construct
VQLQESGGGLVQPGGSLRLSCAASGSNFSINTMGWYRQAPGKQRELVATVTRGGDTNYADSVKGRFTISRDNAKNTVYLQMNSLRPEDTAVYYCHVDEIRESSWTTSNLWGQGTQVTVSSGGGGSGGGGSGGGGSVQLQESGGGLVQPGGSLRLSCAASGSNFRINTMGWYRQAPGKQRELVAPVTRGGSTNYADSVKGRFTISRDNAKNTVYLQMNSLRPEDTAVYYCHVDEVRESSWTTSNLWGQGTQVTVSSGGGGSGGGGSGGGGSVQLQESGGGLVQPGGSLRLSCAASGSSFTINTMGWYRQAPGKQRELVAAVTRGGSTNYAGSVKGRFTISRDNAKNTIYLQMNSLRPEDTAVYYCHVDEIRQSSWTTSNLWGQGTQVTVSS

SEQ ID NO: 132 - protein, 390 aa, synthetic construct
VQLQESGGGLVQPGGSLRLSCAASGSNFRINTMGWYRQAPGKQRELVAPVTRGGSTNYADSVKGRFTISRDNAKNTVYLQMNSLRPEDTAVYYCHVDEVRESSWTTSNLWGQGTQVTVSSGGGGSGGGGSGGGGSVQLQESGGGLVQPGGSLRLSCAASGSNFSINTMGWYRQAPGKQRELVATVTRGGDTNYADSVKGRFTISRDNAKNTVYLQMNSLRPEDTAVYYCHVDEIRESSWTTSNLWGQGTQVTVSSGGGGSGGGGSGGGGSVQLQESGGGLVQPGGSLRLSCAASGSNFSINTMGWYRQAPGKQRELVAALTRGGSTNYADSVKGRFTISRDNAKNTVYLQMNSLRPEDTAVYYCHVDEVRESSWTTSNLWGQGTQVTVSS

SEQ ID NO: 133 - protein, 390 aa, synthetic construct
VQLQESGGGLVQPGGSLRLSCAASGSNFSINTMGWYRQAPGKQRELVATVTRGGDTNYADSVKGRFTISRDNAKNTVYLQMNSLRPEDTAVYYCHVDEIRESSWTTSNLWGQGTQVTVSSGGGGSGGGGSGGGGSVQLQESGGGLVQPGGSLRLSCAASGSNFSINTMGWYRQAPGKQRELVAALTRGGSTNYADSVKGRFTISRDNAKNTVYLQMNSLRPEDTAVYYCHVDEVRESSWTTSNLWGQGTQVTVSSGGGGSGGGGSGGGGSVQLQESGGGLVQPGGSLRLSCAASGSNFRINTMGWYRQAPGKQRELVAPVTRGGSTNYADSVKGRFTISRDNAKNTVYLQMNSLRPEDTAVYYCHVDEVRESSWTTSNLWGQGTQVTVSS

SEQ ID NO: 134 - protein, 525 aa, synthetic construct
VQLQESGGGLAQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSSGGGGSGGGGSGGGGSVQLQESGGGLAQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSSGGGGSGGGGSGGGGSVQLQESGGGLAQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSSGGGGSGGGGSGGGGSVQLQESGGGLAQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSS

SEQ ID NO: 135 - protein, 525 aa, synthetic construct
VQLQESGGGLVQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSSGGGGSGGGGSGGGGSVQLQESGGGLVQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSSGGGGSGGGGSGGGGSVQLQESGGGLVQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSSGGGGSGGGGSGGGGSVQLQESGGGLVQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSS

SEQ ID NO: 136 - protein, 525 aa, synthetic construct
VQLQESGGGLAQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSSGGGGSGGGGSGGGGSVQLQESGGGLAQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSSGGGGSGGGGSGGGGSVQLQESGGGLAQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSS

SEQ ID NO: 137 - protein, 525 aa, synthetic construct
VQLQESGGGLAQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSSGGGGSGGGGSGGGGSVQLQESGGGLAQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSSGGGGSGGGGSGGGGSVQLQESGGGLAQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSS

SEQ ID NO: 138 - protein, 525 aa, synthetic construct
VQLQESGGGLVQPGGSLRLSCVASGSSFSINTMGWYRRAPGKQRELVATVTRDGSTNYANSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCVASGSSFSINTMGWYRRAPGKQRELVATVTRDGSTNYANSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCVASGSSFSINTMGWYRQAPGKQRELVATVTRDGSTNYANSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCVASGSSFSINTMGWYRRAPGKQRELVATVTRDGSTNYANSVKGRFTISRDNAKNTVYLQMNSLRPEDTAVYYCHVDEVRQSSWTTSNLWGQGTQVTVSS

SEQ ID NO: 139 - protein, 525 aa, synthetic construct
VQLQESGGGLVQTGGSLRLSCAASGRAFSLNTMGWYRQAPGKWRELVATVTRGGSTNYAGSVKGRFTISRDNAKNTVYLQMNNLRPEDTAVYYCHVDEVRESSWTTSNLWGQGTQVTVSSGGGGSGGGGSGGGGSVQLQESGGGLVQTGGSLRLSCAASGRAFSLNTMGWYRQAPGKWRELVATVTRGGSTNYAGSVKGRFTISRDNAKNTVYLQMNNLRPEDTAVYYCHVDEVRESSWTTSNLWGQGTQVTVSSGGGGSGGGGSGGGGSVQLQESGGGLVQPGGSLRLSCAASGSIFTINSMGWYRQAPGKWRELVATVTRGGNTNYANSVKGRFTISRDNAKNTVYLQMNTLRPEDTAVYYCHLDEVRDSSWTTSNLWGQGTQVTVSSGGGGSGGGGSGGGGSVQLQESGGGLVQPGGSLRLSCAASGSIFSINSMGWYRQAPGKWRELVATVTRGGSTNYANSVKGRFTISRDNAKNTVYLQMNTLRPEDTAVYYCHLDEVRDSSWTTSNLWGQGTQVTVSS

SEQ ID NO: 140 - protein, 525 aa, synthetic construct
VQLQESGGGLVQTGGSLRLSCAASGRAFSLNTMGWYRQAPGKWRELVATVTRGGSTNYAGSVKGRFTISRDNAKNTVYLQMNNLRPEDTAVYYCHVDEVRESSWTTSNLWGQGTQVTVSSGGGGSGGGGSGGGGSVQLQESGGGLVQPGGSLRLSCAASGSIFTINSMGWYRQAPGKWRELVATVTRGGNTNYANSVKGRFTISRDNAKNTVYLQMNTLRPEDTAVYYCHLDEVRDSSWTTSNLWGQGTQVTVSSGGGGSGGGGSGGGGSVQLQESGGGLVQPGGSLRLSCAASGSIFSINSMGWYRQAPGKWRELVATVTRGGSTNYANSVKGRFTISRDNAKNTVYLQMNTLRPEDTAVYYCHLDEVRDSSWTTSNLWGQGTQVTVSSGGGGSGGGGSGGGGSVQLQESGGGLVQTGGSLRLSCAASGRAFSLNTMGWYRQAPGKWRELVATVTRGGSTNYAGSVKGRFTISRDNAKNTVYLQMNNLRPEDTAVYYCHVDEVRESSWTTSNLWGQGTQVTVSS

SEQ ID NO: 141 - protein, 525 aa, synthetic construct
VQLQESGGGLVQPGGSLRLSCAASGSRFSLNTMGWYRQAPGKQRELVATVTRGGSTNYADSVKGRFTISRDNAKNTVYLQMNSLRPEDTAVYYCHVDELRESGWTTSNLWGQGTQVTVSSGGGGSGGGGSGGGGSVQLQESGGGLVQPGGSLRLSCAASGSRFSLNTMGWYRQAPGKQRELVATVTRGGSTNYADFVKGRFTISRDNAKNTVYLQMNSLRPEDTAVYYCHVDELRESGWTTSNLWGQGTQVTVSSGGGGSGGGGSGGGGSVQLQESGGGLVHSGGSLRLSCAASGSMFSLNTMGWYRQAPGKQRELVATITRGGGTSYADSVKGRFTISRDNAKNTVYLQMNSLRPEDTAVYYCHVDELRQSSWTTSNLWGQGTQVTVSSGGGGSGGGGSGGGGSVQLQESGGGLVQPGGSLRLSCAASGSRFSLNTMGWYRQAPGKQRELVATVTRGGSTNYADFVKGRFTISRDNAKNTVYLQMNSLRPEDTAVYYCHVDELRESGWTTSNLWGQGTQVTVSS

SEQ ID NO: 142 - protein, 525 aa, synthetic construct
VQLQESGGGLVHSGGSLRLSCAASGSMFSLNTMGWYRQAPGKQRELVATITRGGGTSYADSVKGRFTISRDNAKNTVYLQMNSLRPEDTAVYYCHVDELRQSSWTTSNLWGQGTQVTVSSGGGGSGGGGSGGGGSVQLQESGGGLVQPGGSLRLSCAASGSRFSLNTMGWYRQAPGKQRELVATVTRGGSTNYADFVKGRFTISRDNAKNTVYLQMNSLRPEDTAVYYCHVDELRESGWTTSNLWGQGTQVTVSSGGGGSGGGGSGGGGSVQLQESGGGLVQPGGSLRLSCAASGSRFSLNTMGWYRQAPGKQRELVATVTRGGSTNYADFVKGRFTISRDNAKNTVYLQMNSLRPEDTAVYYCHVDELRESGWTTSNLWGQGTQVTVSSGGGGSGGGGSGGGGSVQLQESGGGLVQTGGSLRLSCAASGRAFSLNTMGWYRQAPGKWRELVATVTRGGSTNYAGSVKGRFTISRDNAKNTVYLQMNNLRPEDTAVYYCHVDEVRESSWTTSNLWGQGTQVTVSS

SEQ ID NO: 143 - protein, 525 aa, synthetic construct
VQLQESGGGLAQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCAASGRAFSINTMGWYRQAPGKQRELVATVTRDGSTNYAGSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCAASGSIFIINTMGWYRRAPGKQRELVAAVTRGGSTNYADSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCAASGSIFSINTMGWYRQAPGKQRELVAAVTRGGSTNYADSVKGRFTISRDNAKNTVYLQMNSLRPEDTAVYYCHVDEVRQSSWTTSNLWGQGTQVTVSS

SEQ ID NO: 144 - protein, 525 aa, synthetic construct
VQLQESGGGLVQPGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCAASGSIFSINTMGWYRQAPGKQRELVAAVTRGGSTNYADSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCAASGSIFIINTMGWYRRAPGKQRELVAAVTRGGSTNYADSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCAASGSSFVINTMGWYRQAPGKQRELVAAVTRGGSTNYADSVKGRFTISRDNAKNTVYLQMNSLRPEDTAVYYCHVDEVRQSSWTTSNLWGQGTQVTVSS

SEQ ID NO: 145 - protein, 525 aa, synthetic construct
VQLQESGGGLVQPGGSLRLSCAASGSIFSINTMGWYRQAPGKQRELVAAVTRGGSTNYADSVKGRFTISRDNAKNTVYLQMNSLRPEDTAVYYCHVDEVRQSSWTTSNLWGQGTQVTVSSGGGGSGGGGSGGGGSVQLQESGGGLAQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCAASGRAFSINTMGWYRQAPGKQRELVATVTRDGSTNYAGSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCAASGSIFIINTMGWYRRAPGKQRELVAAVTRGGSTNYADSVKGRFTISRDNAKNTVYLQMNSLRPEDTAVYYCHVDEVRQSSWTTSNLWGQGTQVTVSS

SEQ ID NO: 146 - protein, 525 aa, synthetic construct
VQLQESGGGLVQPGGSLRLSCAASGSIFIINTMGWYRRAPGKQRELVAAVTRGGSTNYADSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCAASGSIFSINTMGWYRQAPGKQRELVAAVTRGGSTNYADSVKGRFTISRDNAKNTVYLQMNSLRPEDTAVYYCHVDEVRQSSWTTSNLWGQGTQVTVSSGGGGSGGGGSGGGGSVQLQESGGGLAQAGGSLRLSCAASGRGFSINTMGWYRQAPGKQRELVAAVTRGGTTNYADSVKGRFTISRDNAKNTVYLQMNSLRPEDTAVYYCHVDEVRQSSWTTSNLWGQGTQVTVSSGGGGSGGGGSGGGGSVQLQESGGGLVQPGGSLRLSCAASGRAFSINTMGWYRQAPGKQRELVATVTRDGSTNYAGSVKGRFTISRDNAKNTVYLQMNSLRPEDTAVYYCHVDEVRQSSWTTSNLWGQGTQVTVSS

SEQ ID NO: 147 - protein, 126 aa, synthetic construct
VQLQESGGGLVQPGGSLRLSCAASGSIFSINLMGWYRQAPGKQRELVATITRGGNTNYAGSVKGRFTISRDNAKNTVYLQMNSLRPEDTAVYYCHVDEVRQSSWTTSNLWGQGTQVTVSSHHHHHH

SEQ ID NO: 148 - protein, 131 aa, synthetic construct
VQLQESGGGLVQPGGSLRLSCAASGSIFSINLMGWYRQAPGKQRELVATITRGGNTNYAGSVKGRFTISRDNAKNTVYLQMNSLRPEDTAVYYCHVDEVRQSSWTTSNLWGQGTQVTVSSGGGGSHHHHHH

SEQ ID NO: 149 - protein, 138 aa, synthetic construct
VQLQESGGGLVQPGGSLRLSCAASGSIFSINLMGWYRQAPGKQRELVATITRGGNTNYAGSVKGRFTISRDNAKNTVYLQMNSLRPEDTAVYYCHVDEVRQSSWTTSNLWGQGTQVTVSSSTPPTPSPSTPPHHHHHH

SEQ ID NO: 150 - protein, 123 aa, synthetic construct
MAQVQLQESGGGLVQPGGSLRLSCAASGSIFSINLMGWYRQAPGKQRELVATITRGGNTNYAGSVKGRFTISRDNAKNTVYLQMNSLRPEDTAVYYCHVDEVRQSSWTTSNLWGQGTQVTVSS

SEQ ID NO: 151 - protein, 121 aa, synthetic construct
QVQLQESGGGLVQPGGSLRLSCAASGSIFSINLMGWYRQAPGKQRELVATITRGGNTNYAGSVKGRFTISRDNAKNTVYLQMNSLRPEDTAVYYCHVDEVRQSSWTTSNLWGQGTQVTVSS

SEQ ID NO: 152 - DNA, 21 bp, synthetic construct
  source 1..21: mol_type = other DNA
cttggtggtcctggctgctct

SEQ ID NO: 153 - DNA, 23 bp, synthetic construct
  source 1..23: mol_type = other DNA
ggtacgtgctgttgaactgttcc

SEQ ID NO: 154 - DNA, 31 bp, synthetic construct
  source 1..31: mol_type = other DNA
tatctagtcgaattccgcccaggtgcagctc

SEQ ID NO: 155 - DNA, 29 bp, synthetic construct
  source 1..29: mol_type = other DNA
agcgactaagcttttgtggttttggtgtc

SEQ ID NO: 156 - DNA, 31 bp, synthetic construct
  source 1..31: mol_type = other DNA
ttccttaacatatggcccaggtgcagctcgt

SEQ ID NO: 157 - DNA, 30 bp, synthetic construct
  source 1..30: mol_type = other DNA
ttaaggaactcgagcacggtgaccagggtc